(19) United States
(12) Patent Application Publication
(10) Pub. No.: US 2005/0095592 A1
(43) Pub. Date: May 5, 2005

(54) Identification of ovarian cancer tumor markers and therapeutic targets

(76) Inventors: Amir A Jazaeri, Charlottsville, VA (US); Jeffrey Boyd, Dobbs Ferry, NY (US); Edison T Liu, Cuscaden Walk (SG)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/505,680

(22) PCT Filed: Feb. 13, 2003
(86) PCT No.: PCT/US03/04688

Related U.S. Application Data
(60) Provisional application No. 60/357,031, filed on Feb. 13, 2002.

(51) Int. Cl. C12Q 1/68; G01N 33/574

(52) U.S. Cl. 435/6; 435/7.23

(57) ABSTRACT

The present disclosure provides methods for classifying ovarian tumors into BRCA1-type, BRCA2-type or non-BRCA-type tumor types by measuring expression levels of a plurality of disclosed ovarian tumor markers. The markers disclosed herein are useful in the diagnosis, staging, detection, and/or treatment of ovarian cancer. Also provided are methods of selecting a treatment regimen by selecting the tumor type. Ovarian cancer-linked logarithmic expression ratios and kits for diagnosis, staging, and detection of ovarian cancer using are also provided.

### PRIORITY CLAIM

This application claims the benefit of U.S. Provisional Application No. 60/357,031, filed Feb. 13, 2002, which is incorporated by reference in its entirety herein.

### FIELD OF THE DISCLOSURE

The present disclosure is related to diagnosing, prognosing, staging, preventing, and treating disease, particularly ovarian cancer.

### BACKGROUND

Ovarian cancer has one of the highest mortality rates of all cancers, due in part to the difficulty of diagnosis. Currently, epithelial ovarian cancer is the leading cause of death resulting from gynecological cancer (see Welsh et al., PNAS 98: 1176-1181, 2001). Studies indicate that the five-year survival rates for ovarian cancer are as follows: Stage 1(93%), Stage 11(70%), Stage III (37%), and Stage IV (25%) (see Holschneider & Berek, Sermin. Surg. Oncol. 19: 3-10, 2000). Thus, there is a particular need for improved methods of early diagnosis, prognosis, and monitoring of ovarian cancer.

Protein and mRNA levels, and changes in these levels, may be associated with specific types of cancer (and cancer progression). Such association is often specific to the type of cancer, meaning that what is overexpressed in one cancer may be under-expressed (or unchanged) in another. Thus, a collection or set of genes/proteins that are differentially regulated in a specific cancer may be indicative and specifically diagnostic of that type of cancer.

Molecular mechanisms involved in the onset and progression of ovarian cancer remain poorly understood. However, some mutations causing ovarian cancer have been identified. Between 5% and 10% of all breast cancers are hereditary. The remaining 90% to 95% are classified as “sporadic,” for which no genetic link to development has been identified.

Breast cancer susceptibility genes BRCA1 (GenBank Accession #U14680) and BRCA2 (GenBank Accession #U43746) are tumor suppressor genes. Germ-line mutations of BRCA1 and BRCA2 are responsible for approximately 5-10% of all epithelial ovarian cancers (see Li and Karlan, Curr. Oncol. Rep. 3:27-32, 2001). Of inherited breast cancers, it is believed that inherited mutations in BRCA1 or BRCA2 are responsible as many as 70% of all cases.

Those with inherited mutations in BRCA1 and BRCA2 have an approximately 63% lifetime risk of developing breast cancer, whereas the general female population has a 12% lifetime risk. The BRCA1 and BRCA2 gene mutations are more often identified in breast cancer patients with poor prognostic factors, which are risk factors that predict for poorer treatment outcomes (e.g., estrogen-receptor-negative tumors, higher growth rates, age less than 35 at onset of disease, breast cancer in both breasts). Development of disease in the opposite breast and ovarian cancer also appear to be more common in breast cancer patients with BRCA1 or BRCA2 mutations. Hence, the presence of BRCA1 or BRCA2 mutations may indicate a need for more aggressive therapeutic treatments.

The alleles of BRCA1 and BRCA2 must be inactivated before tumor development occurs. BRCA1 and BRCA2 are believed to take part in a common pathway involved in maintenance of genomic integrity in cells; however, little is known about the specific molecular mechanisms involved in BRCA mutation associated (BRCA-linked) ovarian carcinogenesis. For example, it is not known whether BRCA1 and BRCA2 mutations affect common or unique molecular pathways in ovarian cancer, or if these pathways overlap with those involved in the formation of sporadic tumors. Both BRCA proteins have been implicated in important cellular functions, including embryonic development, DNA damage repair, and transcriptional regulation (see Scully and Livingston, Nature 408:429-432, 2000; Zheng et al., Oncogene 19:6159-6175, 2000; Welcsh et al., Trends. Genet. 16:69-74, 2000; and MacLachlan et al., J. Biol. Chem. 275:2777-2785, 2000). BRCA1 and BRCA2 have each been implicated in defective homologous recombination DNA repair (see Arvanitis et al., International Journal of Molecular Medicine 10:55-63, 2002), and it is believed that each may be a positive regulator of homologous recombination, with BRCA2 potentially interacting with Rad51, a central homologous recombination effector protein, and BRCA1 regulating GADD45, a DNA damage response gene.

Patients having cervical and endometrial cancer resulting in defects in homologous recombination pathways have been shown to respond favorably to radiotherapy (Arvantis et al.). Therefore, patients having ovarian cancer resulting from a defect in BRCA1 or BRCA2 may similarly benefit from radiotherapy treatment. Hence, the ability to classify ovarian cancer patients into groups based upon the underlying mutation provides advantages in selecting potential courses of treatment, and in deciding whether to pursue a more aggressive course of treatment.

In sum, there is a need to better understand patterns of gene expression that trigger ovarian cancer, as well as downstream genes that may serve as indicators of ovarian cancer progression or as potential tumor suppressors.

### BRIEF SUMMARY OF THE DISCLOSURE

The present disclosure concerns a method of classifying an ovarian tumor as a BRCA1-like or BRCA2-like or non-BRCA-type tumor, by determining a pattern of expression in the ovarian tumor of a plurality of markers listed in Table 1, wherein the pattern of expression in the ovarian tumor is determined relative to a standard ovarian tissue. The pattern of expression of the markers in the ovarian tumor is then compared to the pattern of expression of the same markers in tissue from a known BRCA1-like or BRCA2-like or non-BRCA-type tumor. A similarity of the pattern of expression in the ovarian tumor to a pattern of expression of the comparison tissue of the known BRCA1-like tumor classifies the ovarian tumor as a BRCA1-like tumor; a similarity of the pattern of expression in the ovarian tumor to a pattern of expression of the known BRCA2-like tumor classifies the ovarian tumor as a BRCA2-like tumor; and a similarity of the pattern of expression in the ovarian tumor to a pattern of expression of the known sporadic tumor classifies the ovarian tumor as a sporadic tumor.

The patterns of expression are determined, for example, by determining a pattern of over-expression or under-expression of the plurality of markers in the ovarian tumor to over-expression or under-expression of the plurality of markers of the comparison tissue. Alternatively, a pattern of both over-expression and under-expression of the plurality of markers in the ovarian tumor is compared to over-expression and under-expression of the plurality of markers in the comparison tissue.

It has also been discovered that ovarian tumors that do not contain a BRCA1 or BRCA2 mutation may be BRCA-1-like or BRCA2-like in that the pattern of expression of the markers is similar to a tumor having a BRCA-1 or BRCA-2 mutation. Hence tumors that would otherwise be considered “non-BRCA-type” can be classified as BRCA-1-like or BRCA-2-like, which can contribute to decisions about treatment and prognosis even in the absence of the mutation.

Standard ovarian tissue serves as a baseline from which patterns of over expression and under expression can be determined. The “standard” ovarian tissue may be, for example, from an immortalized ovarian cell, ovarian tissue from a subject not having ovarian cancer, a subject not predisposed to developing ovarian cancer, or ovarian tissue from a subject from whom the ovarian tumor was obtained at an earlier point in time. It is also possible for the standard tissue to be tumor tissue taken from a patient at an earlier point in time, for example prior to treatment (for example prior to the administration of chemotherapy). However in most instances the “standard” tissue is “normal” non-tumor ovarian tissue, such as an immortalized ovarian cell line, for example an IOSE cell line.

Many different approaches are described in this disclosure for determining the patterns of expression, and assessing similarities. In specific examples, the patterns of expression are patterns of logarithmic expression ratios, hierarchical clustering patterns, or multidimensional scaling patterns. The patterns may be compared visually or statistically to arrive at conclusions regarding similarity of the patterns. For example, when a multi-dimensional scaling pattern is used to generate a three-dimensional representation of data clusters associated with BRCA1-like, BRCA2-like or non-BRCA-like tumors, the position of a data point obtained from the tumor specimen that is being analyzed can indicate whether the tumor specimen has a pattern of expression associated with one of these groups. If the data point from the tumor specimen is present within or closely associated with one of these clusters, it is assigned a classification the same as the cluster in which is it contained or with which it is associated.

Another approach to comparing patterns of over expression and under expression is to assign different color intensities to standard normal deviation values of the logarithmic expression ratios. Similarities of color patterns can then be used to arrive at a qualitative assignment of a tumor specimen to a classification. In another approach, the logarithmic expression ratios of the plurality markers is compared using compound covariate predictor analysis.

In particular examples discussed herein, a BRCA1-like ovarian tumor is differentiated from a non-BRCA-like ovarian tumor by comparing relative logarithmic expression ratios of at least one marker shown in Table 6. In more particular embodiments, the pattern of expression of all the markers in Table 6 (CD72, SLC25A11, LCN2, PSTP1P1, SIAHBP1, UBE1, WAS, IDH2, and PC7K1) is compared to the pattern of expression of these same markers in the specimen undergoing classification.

In another example, a BRCA2-like ovarian tumor is distinguished from a non-BRCA-like ovarian tumor by comparing relative logarithmic expression ratios of at least one marker shown in Table 7, and in some embodiments both of the markers (LOC51760 and LRPAP1). In yet other examples BRCA1- and BRCA2-like ovarian tumors are distinguished from non-BRCA-like ovarian tumors by comparing relative logarithmic expression ratios of at least one marker shown in Table 8, for example PSTP1P1, IDH2, and PCTK1, or all the markers in Table 8. In other examples, a BRCA1-like ovarian tumor is distinguished from a BRCA2-like ovarian tumor by comparing relative logarithmic expression ratios of at least one marker shown in Table 10, more than one marker shown in Table 10, or all the markers in Table 10.

The disclosed methods also include selecting a treatment strategy based on classifying the ovarian tumor as BRCA1-like, BRCA2-like or non-BRCA-like. For example, the treatment strategy may include selecting a more aggressive treatment regimen for a BRCA1-like or BRCA2-like tumor (even if the tumor does not contain a BRCA1 or BRCA2 mutation). Such treatment regimens can include chemotherapy, radiotherapy, or surgical removal of the tumor and/or surrounding tissue.

In yet other disclosed examples, the expression patterns of a tumor specimen and known comparison tissue are compared using a database of patterns (for example a database of logarithmic expression patterns) associated with BRCA1-like, BRCA2-like or non-BRCA-like ovarian tumors. The database can contain, for example, expression ratios of the plurality of markers in standard tissue. The patterns of the expression ratios of the plurality of markers of the tumor specimen can then be compared to the pattern of expression ratios of the same markers in the standard tissue.

In some examples, comparisons may be made just of patterns of over expression, for one or more markers that is over expressed as listed in Table 5. Alternatively, comparisons may be made just of patterns of under expression. The patterns of expression may be obtained by using nucleic acid sequences of the markers to perform nucleic acid hybridization of specific oligonucleotide probes to the nucleic acid sequences. The markers may be amplified prior to performing nucleic acid hybridization, and expression quantitated to detect a level of differential expression. The markers are conveniently provided on an array, such as a cDNA microarray. In one example the cDNA microarray contains at least 50, 100, 200, 400 or more of the markers listed in Table 1.

The results of these comparisons can be used to diagnose or provide a prognosis of progression of ovarian cancer in a subject. The patterns of expression can also be used to screen for therapeutic agents for the treatment of ovarian cancer, or monitoring response to therapy in a subject, by looking for a return of the patterns of expression of the ovarian tumor toward a non-tumor tissue pattern. Kits are also provided for performing these analyses, and the kit may include arrays with cDNAs of the markers.

### BRIEF DESCRIPTION OF THE FIGURES

FIG. 1 shows the overall expression differences between BRCA1-like, BRCA2-like, and non-BRCA-like ovarian epithelial cancers. FIG. 1A. Multidimensional scaling model based on the overall gene expression (6,445 filtered spots, Example 1) in BRCA1-linked (solid circles), BRCA2-linked (open circles), and sporadic tumors (asterisks). FIG. 1B. The magnitude of differences in gene expression between various tumor groups as revealed by the number of genes differentially expressed among them using the uniform statistical cutoff P<0.0001.

FIG. 2 illustrates that BRCA1- and BRCA2-discriminating genes also segregate sporadic ovarian cancers into two groups (BRCA1-like and BRCA2-like). FIG. 2A. Hierarchical clustering of 110 non-redundant genes (see Table 9, Addendum) showing significant differential expression between BRCA1-linked and (B1) and BRCA2-linked (B2) tumors (modified F-test P<0.0001). The red and green color intensities represent standard normal deviation (Z-score) values from the mean expression level of each gene (represented as black) across sixty-one tumor samples (Example 1). FIG. 2A′ is a duplication of FIG. 2A, but is printed in grey tones rather than in color. FIG. 2B. Hierarchical clustering of sporadic and BRCA-linked tumor samples based on the expression pattern of the 110 BRCA-discriminating patterns of gene expression. The B-, B2-, and C-labeled samples signify BRCA1-linked, BRCA2-linked, and sporadic tumors, respectively. FIG. 2C. Hierarchical clustering of sporadic samples in the absence of BRCA-linked tumors reveals two major clusters corresponding to BRCA1-type and BRCA2-type patterns of gene expression.

FIG. 3 shows molecular profiles of sixty-one tumors as defined by the genes whose expression significantly differentiated BRCA1 and BRCA2 tumors (P<0.0001) (see Example 1, and Table 9). The red and green color intensities represent expression levels shown as standard normal deviation (Z-score) values from the mean expression level of each gene (represented as black) across sixty-one tumor samples. The genes are numbered consecutively 1-61 in FIG. 3A, and 62-116 in FIG. 3B. FIG. 3A′ and FIG. 3B′ are duplications of FIG. 3A and FIG. 3B, respectively, but are printed in grey tones rather than in color. FIG. 3C shows the correlation of the designated rows to genes and SEQ ID NOs for the molecular profile in FIG. 3A and FIG. 3D shows the correlation of the designated rows to genes and SEQ ID NOs for the molecular profile in FIG. 3B.

FIG. 4 shows gene expression differences between BRCA-linked and sporadic tumors. A modified F-test with a statistical significance level of P<0.0001 was used to evaluate genes differentially expressed between tumor types. The red and green color intensities represent expression levels shown as standard normal deviation (Z-score) values from the mean expression level of each gene (represented as black) across all sixty-one tumor samples. Each gene name is followed by the corresponding I.M.A.G.E. clone number spotted on the array. FIG. 4A. Genes differentially expressed between BRCA1-linked (B) and sporadic (C) samples. Genes located on Xp11 appear in red. FIG. 4B. Examples of genes differentially expressed between BRCA2-linked (B2) and sporadic (C) samples. FIG. 4C. Examples of differentially expressed genes between the combined BRCA1- and BRCA2-linked group (B and B2, respectively) and the sporadic (C) samples. FIG. 4A-C′ is a duplication of FIG. 4A-C, but is printed in grey tones rather than in color. FIG. 4D BRCA1-linked tumors exhibit significantly higher expression levels (P<0.001) of all six genes mapped to Xp11.23 compared to the sporadic cancers. Error bars reflect standard error.

FIG. 5 is a bar graph showing an evaluation of gene expression patterns common to BRCA-linked and sporadic tumors. FIG. 5A shows the expression of twenty-five genes that showed two-fold or greater down-regulation as compared to the IOSE reference cell line. FIG. 5B shows the expression of twenty-five genes that showed two-fold or greater up-regulation as compared to the IOSE reference cell line. Error bars reflect standard error. (FOS, HE4 and CD24) have been previously reported to be overexpressed in ovarian cancers. Several of the overexpressed genes that have been demonstrated to be interferon-responsive are presented in italics. The * symbol denotes immediate-early response genes.

FIG. 6 is a series of bar graphs illustrating semi-quantitative RT-PCR (sqRT-PCR) analysis of gene expression confirms the cDNA microarray data. Expression patterns of select genes were examined using sqRT-PCR in representative BRCA1-linked (bars 1-5), BRCA2-linked (bars 6-10), and sporadic (bars 11-15) samples. The expression level of each gene in the tumor samples was compared to those of normal postmenopausal ovary (N) and the reference IOSE cells (R). All data has been normalized to β-actin is presented as fold expression compared to the IOSE reference RNA. FIG. 6A shows results for genes HE4, RSG1, and CD74. FIG. 6B shows results for genes ZFP36, TOP2A and HLA-DRB1.

### BRIEF DESCRIPTION OF THE TABLES

TABLE 1 (see Addendum) lists 822 ovarian cancer-related nucleic acid molecules that show altered expression in ovarian cancer. The nucleic acids are identified by their SEQ ID NO, their gene name (if one has been assigned), the I.M.A.G.E Clone ID number associated with the nucleic acid sequence, the UniGene number (if one has been assigned), and a description of the gene (if known). Because more than one GenBank Accession Number is sometimes provided for a given nucleic acid molecule, the Table groups the SEQ ID NO assigned to each GenBank Accession Number with nucleic acid molecule. For example, the entry for BCKDHB in Table 1 provides SEQ ID NOs: 16-17 (represented by GenBank Accession number AA427739 and GenBank Accession number AA434304). Each of the 822 SEQ ID NOs are included in the attached sequence listing.

TABLE 2 catalogs the clinicopathologic features of the tumor samples in a study of sixty-one cases of pathologically-confirmed epithelial ovarian adenocarcinoma.

TABLE 3 lists representative gene-specific primer sequences used to amplify RNA for analysis by semi-quantitative PCR.

TABLE 4 (see Addendum) lists markers that were under-expressed in ovarian cancer in a comparison of ovarian epithelial cancer cells to normal postmenopausal ovarian tissue.

TABLE 5 (see Addendum) lists markers that were ove-rexpressed in ovarian cancer in a comparison of ovarian epithelial cancer cells to normal postmenopausal ovarian tissue.

TABLE 6 (see Addendum) lists markers that were differentially expressed between BRCA1-linked and sporadic tumors in a comparison to reference immortalized ovarian epithelial cells.

TABLE 7 (see Addendum) lists markers that were differentially expressed between BRCA2-linked and sporadic tumors in a comparison to reference immortalized ovarian epithelial cells.

TABLE 8 (see Addendum) lists markers that were differentially expressed between combined BRCA1-linked and BRCA2-linked versus sporadic tumors in a comparison to reference immortalized ovarian epithelial cells.

TABLE 9 (see Addendum) lists markers that were differentially expressed between BRCA 1-linked and BRCA2-linked tumors in a comparison to reference immortalized ovarian epithelial cells.

TABLE 10 (see Addendum) lists markers that can be used to segregate BRCA1-like from BRCA2-like tumor types using compound covariate prediction analysis.

TABLE 11 (see Addendum) lists the results of compound covariate predictor analysis for the sixty-one tumors disclosed herein, analyzed using the markers in Table 10.

### BRIEF DESCRIPTION OF THE SEQUENCE LISTING

The nucleic and amino acid sequences listed in the accompanying sequence listing are shown using standard letter abbreviations for nucleotide bases, and single letter code for amino acids, as defined in 37 C.F.R. § 1.822. Only one strand of each nucleic acid sequence is shown, but the complementary strand is understood as included by any reference to the displayed strand. In the accompanying sequence listing:

SEQ ID NO: 1 is a 63-nucleotide synthetic primer containing a T7 RNA polymerase binding site.

SEQ ID NOs: 2 and 3 are ACTB gene-specific primers used for amplification during semi-quantitative RT-PCR.

SEQ ID NOs: 4 and 5 are HE4 gene-specific primers used for amplification during semi-quantitative RT-PCR.

SEQ ID NOs: 6 and 7 are ZFP36 gene specific primers used for amplification during semi-quantitative RT-PCR.

SEQ ID NOs: 8 and 9 are RGS1 gene specific primers used for amplification during semi-quantitative RT-PCR.

SEQ ID NOs: 10 and 11 are CD74 gene specific primers used for amplification during semi-quantitative RT-PCR.

SEQ ID NOs: 12 and 13 are TOP2A gene specific primers used for amplification during semi-quantitative RT-PCR.

SEQ ID NOs: 14 and 15 are HLA-DRB1 gene specific primers used for amplification during semi-quantitative RT-PCR.

SEQ ID NOs: 16 through 822 are ovarian cancer-related nucleic acid molecules that show altered expression in ovarian cancer. These nucleic acid molecules are listed in Table 1, and their sequence information is provided in the attached sequence listing.

### DETAILED DESCRIPTION

1. Abbreviations

cDNA: complementary DNA

DNA: deoxyribonucleic acid

ELISA: enzyme-linked immunosorbent assay

EST: expressed sequence tag

I.M.A.G.E.: Integrated Molecular Analysis of Genomes and their Expression Consortium

IOSE: immortalized ovarian surface epithelial cell lines

MDS: multidimensional scaling

PCR: polymerase chain reaction

RIA: radioimmunoassay

RNA: ribonucleic acid

RT-PCR: reverse transcription-polymerase chain reaction

siRNA: small inhibitory RNA molecule

sqRT-PCR: semi-quantitative reverse transcription-polymerase chain reaction

STS: sequence-tagged site

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In accordance with the present disclosure, conventional molecular biology, microbiology, and recombinant DNA techniques within the skill of the art are used. Such techniques are fully explained in the literature (see, e.g., Sambrook et al., 1989. Molecular cloning, a laboratory manual. 2^{nd} ed. Cold Spring Harbor Laboratory, Cold spring Harbor, N.Y.; Glover, 1985, DNA Cloning: A practical approach, volumes I and II oligonucleotide synthesis, MRL Press, LTD., Oxford, U.K.; Hames and Higgins, 1985, Transcription and translation; Hames and Higgins, 1984, Animal Cell Culture; Freshney, 1986, Immobilized Cells And Enzymes, IRL Press; and Perbal, A Practical Guide to Molecular Cloning, John Wiley & Sons, New York, 1988).

In order to facilitate review of the various embodiments of the invention, the following explanations of specific terms are provided:

Altered expression or differential expression refers to expression of a nucleic acid (e.g., mRNA or protein) in a subject or biological sample from a subject that deviates from that expression in a subject or biological sample from a subject having normal (wild-type) characteristics for the biological condition associated with the nucleic acid. Normal expression can be found in a control, a standard for a population, etc. For instance, where the altered expression manifests as a diseased condition, such as growth of a tumor or neoplasia or onset of a cancer such as ovarian cancer, characteristics of normal expression might include an individual who is not suffering from the condition (e.g., a subject not displaying neoplasia growth or not having ovarian cancer), a population standard of individuals believed not to be suffering from the disease, etc. For instance, certain altered expression (such as altered expression of a BRCA nucleic acid), can be described as being associated with the biological conditions of altered (e.g., over-expressed or under-expressed) nucleic acid expression and a tendency to develop gynecological cancer, such as ovarian cancer. Likewise, altered expression may be associated with a disease. The term “associated with” includes an increased risk of developing the disease.

Controls or standards (e.g., a reference cell line, such as immortalized epithelial ovarian cells) for comparison to a sample (e.g., an ovarian cancer tumor), for the determination of altered expression, include samples believed to be normal for the studied characteristic, as well as laboratory values, even though possibly arbitrarily set, keeping in mind that such values may vary from laboratory to laboratory. Laboratory standards and values may be set based on a known or determined population value and may be supplied in the format of a graph or table that permits easy comparison of measured, experimentally determined values.

When used in reference to a nucleic acid, amplification includes techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

An array is an arrangement of molecules, particularly biological macromolecules (such as polypeptides or nucleic acids) or cell or tissue samples, in addressable locations on or in a substrate. The array may be regular (arranged in uniform rows and columns, for instance) or irregular. The number of addressable locations on the array can vary, for example from a few (such as three) to more than 50, 100, 200, 500, 1000, 10,000, or more. A microarray is an array that is miniaturized so as to require or be aided by microscopic examination for evaluation or analysis. A cDNA microarray is an array of multiple cDNA molecules, fixed in addressable locations, to which complementary nucleic acids in applied samples may hybridize (see Hegde et al., Biotechniques 29(3): 548-562, 2000). cDNA microarrays of the disclosure provide for qualitative and quantitative analysis of gene expression of the molecules contained in the array.

Within an array, each arrayed sample (feature) is addressable, in that its location can be reliably and consistently determined within the at least two dimensions of the array. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (e.g., in radially distributed lines, spiral lines, or ordered clusters). Addressable arrays usually are computer readable, in that a computer can be programmed to correlate a particular address on the array with information about the sample at that position (e.g., expression data, including for instance signal intensity as well as the identity of the sample). In some examples of computer readable formats, the individual features in the array are arranged regularly, for instance in a Cartesian grid pattern, which can be correlated to address information by a computer.

The sample application location on an array (the “feature”) may assume many different shapes. Thus, though the term “spot” may be used herein, it refers generally to a localized placement of molecules or tissue or cells, and is not limited to a round or substantially round region. For instance, substantially square regions of application can be used with arrays encompassed herein, as can be regions that are, for example, substantially rectangular, triangular, oval, irregular, or another shape. Within a single array, feature shapes do not usually vary, though they will in some embodiments.

In certain example arrays, one or more features will occur on the array a plurality of times (e.g., twice) to provide internal controls.

A biological sample is any sample in which the presence of a protein and/or ongoing expression of a protein may be detected. Suitable biological samples include samples containing genomic DNA or RNA (including mRNA), obtained from body cells of a subject, such as but not limited to those present in peripheral blood, urine, saliva, cells obtained by pap smear, sera, tissue biopsy, surgical specimen, amniocentesis samples and autopsy material.

A BRCA1-like tumor is a tumor in which the gene expression pattern is substantially similar to the gene expression pattern in a tumor from a subject who has a mutation in BRCA1. Similarly, a BRCA2-like tumor is a tumor in which the gene expression pattern is substantially similar to the gene expression pattern in a tumor from a subject who has a mutation in BRCA2. As described herein, sporadic tumors may share gene expression patterns with BRCA-linked and or BRCA2-linked tumors. Hence, sporadic and other tumors (such as tumors for which no BRCA genetic test has been conducted) that have gene expression patterns similar to a BRCA1-linked tumor are “BRCA1-like” tumors.

A cancer is a biological condition in which a malignant tumor or other neoplasm has undergone characteristic anaplasia with loss of differentiation, increased rate of growth, invasion of surrounding tissue, and/or which is capable of metastasis.

The term cancer includes ovarian cancer, such as ovarian epithelial cancer, which originates in the ovaries and may manifest as epithelial tumors, germ cell tumors, or stromal tumors. Also included are different stages of a single cancer, for instance both primary and recurrent ovarian cancer, and cancer at any progressive stage, such as Stages I-IV. Ovarian cancer is considered a gynecological cancer.

A subject may be classified into an ovarian cancer stage based upon evaluation of a biological sample from the subject for indices known in the art or disclosed herein as being indicative of that stage of ovarian cancer. For example, a subject may be classified as having a cancer state of cancer-free, active ovarian cancer (i.e., stage I, II, III, or IV ovarian cancer), or in remission from previous ovarian cancer.

cDNA is a piece of DNA lacking internal, non-coding segments (introns) and regulatory sequences that determine transcription. cDNA is generally synthesized in the laboratory by reverse transcription from messenger RNA extracted from cells.

Compound covariate prediction analysis is a method of predicting into which of two groups a sample will be assinged using a given statistical signficance cutoff (e.g., P<0.0005). The method creates a multivariate predictor for one of two classes to each sample and includes in the multivariate predictor only those components (e.g., nucleic acids expressing on a cDNA microarray) that meet the statistical signficance cutoff. The multivariate predictor is a weighted linear combination of logarithmic ratios for components that are univariately significant. The weight consists of the univariate t-statistics for comparing the classes.

DNA is a polymer that comprises the genetic material of most living organisms (some viruses have genomes comprising RNA). The repeating units in most natural DNA polymers are four different nucleotides, each of which comprises one of the four bases, adenine, guanine, cytosine and thymine, bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. Thus, for instance, it is appropriate to generate primers from the reverse complement sequence of the disclosed nucleic acid molecules.

An expressed sequence tag (EST) is a unique stretch of DNA within a coding region of a gene that is useful for identifying full-length genes and serves as a landmark for gene mapping. An EST is a sequence tagged site (STS) derived from cDNA.

Expression of a gene is the process by which the coded information of a gene is converted into an operational or non-operational part of a cell, often including the synthesis of a protein. Gene expression can be influenced by external signals. For instance, exposure of a cell to a hormone may stimulate expression of a hormone-induced gene. Different types of cells may respond differently to an identical signal.

Expression of a gene also may be regulated in the pathway from DNA to RNA to protein. Ways in which regulation occurs include through controls acting on transcription, translation, RNA transport and processing, degradation of intermediary molecules such as mRNA, or through activation, inactivation or compartmentalization or degradation of specific protein molecules after they have been made.

Changes in gene expression may be associated with specific types of cancer (and cancer progression). Such association is fairly specific to the type of cancer, and thus what is overexpressed in one cancer may be underexpressed (or unchanged) in another.

The expression of several genes may be grouped into an expression pattern or expression profile. Such patterns or profiles may be unique to an individual sample depending upon certain factors, for instance biological stimuli introduced into the subject from which the sample was taken (e.g., a hormone) or ongoing disease within the subject (e.g., ovarian cancer). Thus, a collection or set of genes/proteins that are differentially regulated in a specific cancer may be indicative and specifically diagnostic of that type of cancer. In addition, specific expression patterns may indicate particular mutations within the individual that correlate and/or cause the disease, for instance a mutation in BRCA1 or BRCA2, or may indicate a larger class of disease, such as a BRCA1-like or BRCA2-like cancer. Furthermore, changing the expression patterns of these genes to restore the normal state, or bring the condition closer to the normal state in one or more characteristic, may constitute a treatment for cancer.

As disclosed herein, the expression pattern of an unknown tumor may be compared to the expression pattern of known BRCA1-linked and BRCA2-linked markers to determine if the expression patterns are sufficiently similar to classify the unknown as a BRCA1-like or BRCA2-like tumor.

Gene amplification or genomic amplification is an increase in the copy number of a gene or a fragment or region of a gene or associated 5′ or 3′ region, as compared to the copy number in normal tissue. An example of a genomic amplification is an increase in the copy number of an oncogene. A “gene deletion” is a deletion of one or more nucleic acids normally present in a gene sequence and, in extreme examples, can include deletions of entire genes or even portions of chromosomes.

A gene expression fingerprint (or profile) is a distinct or identifiable pattern of gene expression, for instance a pattern of high and low expression of a defined set of genes or gene-indicative nucleic acids such as ESTs; in some instances, as few as one or two genes may provide a profile, but often more genes are used in a profile, for instance at least three, at least 5, at least 10, at least 20, at least 25, or at least 50 or more. Gene expression fingerprints (also referred to as profiles) can be linked to a tissue or cell type, to a particular stage of normal tissue growth or disease progression, or to any other distinct or identifiable condition that influences gene expression in a predictable way. Gene expression fingerprints can include relative as well as absolute expression levels of specific genes, and often are best viewed in the context of a test sample compared to a baseline or control sample fingerprint. By way of example, a gene expression profile may be read on an array (e.g., a polynucleotide or polypeptide array). Arrays are now weIl known, and for instance gene expression arrays have been previously described in published PCT application number WO9948916 (“Hypoxia-Inducible Human Genes, Proteins, and Uses Thereof”), incorporated herein by reference in its entirety.

As disclosed herein, the gene expression profile of an unknown tumor may be compared for similarities and differences to the expression profile of a tumor known to express in a BRCA-like manner (e.g., a BRCA1-like or BRCA2-like tumor).

A genomic target sequence is a sequence of nucleotides located in a particular region in the human genome that corresponds to one or more specific genetic abnormalities, such as a nucleotide polymorphism, a deletion, or amplification. The target can be for instance a coding sequence; it can also be the non-coding strand that corresponds to a coding sequence.

Gynecological cancers are cancers of the female reproductive system, and include cancers of the uterus (e.g., endometrial carcinoma), cervix (e.g., cervical carcinoma), ovaries (e.g., ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (e.g., squamous cell carcinoma, intraepithelial carcinoma, adenocarcinoma, fibrosarcoma, melanoma), vagina (e.g., clear cell carcinoma, squamous cell carcinoma, botryoid sarcoma), embryonal rhabdomyosarcoma, and fallopian tubes (e.g., carcinoma).

An isolated biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins that have been isolated thus include nucleic acids and proteins purified by standard purification methods. The term also embraces nucleic acids, peptides and proteins prepared by recombinant expression in a host cell as well as chemically synthesized nucleic acids.

A marker is a diagnostic indicator of disease. A marker may consist of any signal indicating the presence of the disease, e.g., a physiological change in the body of a subject or increased or decreased levels of a substance such as a protein correlated to the disease. Markers are often found in body fluid samples from a subject. By way of example, prostate specific antigen is a tumor marker used to detect progression of prostate cancer. The molecules disclosed herein, for instance in Table 1 are useful as tumor markers for diagnosing, prognosing, staging, preventing, and treating cancerous disease, such as ovarian cancer.

A mutation includes any change of the DNA sequence within a gene or chromosome. In some instances, a mutation will alter a characteristic or trait (phenotype), but this is not always the case. Types of mutations include base substitution point mutations (e.g., transitions or transversions), deletions, and insertions. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

This term specifically encompasses variations that arise through somatic mutation, for instance those that are found only in disease cells, but not constitutionally. in a given individual. Examples of such somatically-acquired variations include the point mutations that frequently result in altered function of various genes that are involved in development of cancers. This term also encompasses DNA alterations that are present constitutionally, that alter the function of the encoded protein in a readily demonstrable manner, and that can be inherited by the children of an affected individual. In this respect, the term overlaps with “polymorphism,” as defined below, but generally refers to the subset of constitutional alterations that have arisen within the past few generations in a kindred and that are not widely disseminated in a population group. In particular embodiments, the term is directed to those constitutional alterations that have major impact on the health of affected individuals, such as those resulting in onset of a disease such as a gynecological cancer.

An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

A neoplasm is a new and abnormal growth, particularly a new growth of tissue or cells in which the growth is uncontrolled and progressive. A tumor is an example of a neoplasm.

A non-BRCA-type tumor is a tumor in which the gene expression pattern of the BRCA1-linked and BRCA2-linked markers disclosed in Table 1 is not similar to either a BRCA1-like or BRCA2-like gene expression pattern.

A nucleic acid is a deoxyribonucleotide or ribonucleotide polymer in either single or double stranded form, and unless otherwise limited, encompasses known analogues of natural nucleotides that hybridize to nucleic acids in a manner similar to naturally occurring nucleotides.

A nucleic acid sequence (or polynucleotide) is a DNA or RNA molecule, and includes polynucleotides encoding full-length proteins and/or fragments of such full length proteins which can function as a therapeutic agent.

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

An ovarian cancer-related molecule includes nucleic acids (such as DNA or RNA or cDNA) and proteins that are altered (for example by mutation or abnormal expression) in ovarian cancer.

Pharmaceutically acceptable carriers include compositions and formulations suitable for pharmaceutical delivery of the nucleotides and proteins herein disclosed. Martin, Remington's Pharmaceutical Sciences, published by Mack Publishing Co., Easton, Pa., 19th Edition, 1995, describes conventional pharmaceutically acceptable carriers.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Primers are short nucleic acids, preferably DNA oligonucleotides 10 nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Primers as used in the present disclosure preferably comprise at least 10 nucleotides of the nucleic acid sequences that are shown to encode specific proteins. In order to enhance specificity, longer primers may also be employed, such as primers that comprise 15, 20, 30, 40, 50, 60, 70, 80, 90 or 100 consecutive nucleotides of the disclosed nucleic acid sequences. Methods for preparing and using probes and primers are described in the references, for example Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences; Innis et al. (1990) PCR Protocols, A Guide to Methods and Applications, Innis et al. (Eds.), Academic Press, San Diego, Calif. PCR primer pairs can be derived from a known sequence, for example, by using computer programs intended for that purpose such as Primer (Version 0.5, 1991, Whitehead Institute for Biomedical Research, Cambridge, Mass.).

When referring to a primer, the term specific for (a target sequence) indicates that the primer hybridizes under stringent conditions substantially only to the target sequence in a given sample comprising the target sequence.

A probe comprises an isolated nucleic acid attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., Sambrook et al. (In Molecular Cloning, A Laboratory Manual, CSHL, New York, 1989) and Ausubel et al. (In Current Protocols in Molecular Biology, John Wiley & Sons, New York, 1998).

A protein is a biological molecule expressed by a gene and comprised of amino acids.

A purified molecule is one that has been purified relative to its original environment. The term “purified” does not require absolute purity; rather, it is intended as a relative term. Thus, for example, a purified protein preparation is one in which the protein referred to is more pure than the protein in its natural environment within a cell or within a production reaction chamber (as appropriate). Non-limiting examples of purified molecules are those that are 50%, 75%, or 90% pure.

A recombinant nucleic acid is a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques such as those described in Sambrook et al., In Molecular Cloning: A Laboratory Manual, CSHL, New York, 1989. The term recombinant includes nucleic acids that have been altered solely by deletion of a portion of the nucleic acid. For instance, a plasmid is recombinant if some portion of the naturally occurring plasmid has been deleted. Equally, if the sequence of such a plasmid has been altered, for example by a nucleotide substitution (or addition or deletion), that plasmid is said to be recombinant.

Sequence identity is the similarity between two nucleic acid sequences, or two amino acid sequences is expressed in terms of the similarity between the sequences, otherwise referred to as sequence identity. Sequence identity is frequently measured in terms of percentage identity (or similarity or homology); the higher the percentage, the more similar are the two sequences. Methods of alignment of sequences for comparison are well-known in the art. Various programs and alignment algorithms are described in: Smith and Waterman, J. Theor. Biol. 91(2): 379-380, 1981; Needleman and Wunsch, J. Mol. Bio. 48:443-453, 1970; Pearson and Lipman, Methods in Molec. Biology 24: 307-331, 1988; Higgins and Sharp, Gene 73:237-244, 1988; Higgins and Sharp, CABIOS 5:151-153, 1989; Corpet et al., Nucleic Acids Research 16:10881-10890, 1988; Huang et al., Computer Applications in BioSciences 8:155-165,1992; and Pearson et al., Meth. Mol. Bio. 24: 307-331,1994. Altschul et al., Nat. Genet. 6(2): 119-129, 1994 presents a detailed consideration of sequence alignment methods and homology calculations.

The NCBI Basic Local Alignment Search Tool (BLAST) (see Altschul et al. J. Mol. Biol. 215: 403-410, 1990) is available from several sources, including the National Center for Biotechnology Information (NCBI, Bethesda, Md.) and on the Internet, for use in connection with the sequence analysis programs blastp, blastn, blastx, tblastn and tblastx. The Search Tool can be accessed at the NCBI website, together with a description of how to determine sequence identity using this program.

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid molecules that all encode substantially the same protein.

Serial analysis of gene expression (SAGE) is the use of short diagnostic sequence tags to allow the quantitative and simultaneous analysis of a large number of transcripts in tissue, as described in Velculescu et al., Science 270:484-487, 1995.

A standard is a reference against which a value (e.g., level of expression of a marker) can be compared. By way of example, a non-cancerous cell line may be used as a standard for comparing the level of expression of tumor markers in an ovarian tumor sample. Non-limiting examples of standards useful with the disclosed methods of analysis of patterns of expression of markers include a non-cancerous sample (e.g., normal ovarian tissue), a sample from a subject prior to development of a cancer or at an earlier stage of the cancer, and a cell line (e.g., immortalized ovarian epithelial cells, such as IOSE cells) considered to display wild-type expression levels of the markers. In some embodiments, a reference RNA is arbitrarily chosen, but used consistently in relation to all tumor samples.

A subject is a living multi-cellular vertebrate organisms, a category that includes both human and non-human mammals.

A therapeutic agent, as used in a generic sense, is a composition used for treating a subject, such as a pharmaceutical or prophylactic agent.

A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Treating a disease includes inhibiting or preventing the partial or full development or progression of a disease (e.g., ovarian cancer), for example in a person who is known to have a predisposition to a disease. An example of a person with a known predisposition is someone having a history of breast or ovarian cancer in his or her family, or who has been exposed to factors that predispose the subject to a condition, such as exposure to radiation. Furthermore, treating a disease refers to a therapeutic intervention that ameliorates at least one sign or symptom of a disease or pathological condition, or interferes with a pathophysiological process, after the disease or pathological condition has begun to develop.

In some aspects, a more aggressive treatment may be selected if warranted. By way of example, if a subject is found to have a BRCA1-like or BRCA2-like gene expression pattern, a more aggressive treatment, such as chemotherapy, radiotherapy, or surgical removal of the affected tissue and/or surrounding area may be selected.

A tumor is an abnormal mass of tissue, or neoplasm that may be either malignant or non-malignant. “Tumors of the same tissue type” refers to primary tumors originating in a particular organ (such as breast, ovary, bladder or lung). Tumors of the same tissue type may be divided into tumor of different sub-types, for example ovarian carcinomas can be further classified based on tumor histology as adenocarcinoma, serous, endometrial, clear cell or mixed. Tumors may also be classified according to a genetic abnormality associated with the development of that type of tumor. By way of example, a tumor associated with a defect in tumor suppressor genes BRCA1 or BRCA2 is referred to herein as a “BRCA1- or BRCA2-linked” tumor. As described herein, a sporadic ovarian tumor is a tumor arising for a reason other than a mutation in BRCA1 or BRCA2. However, the similarities in the pattern of expression of ovarian cancer markers in sporadic tumors to those in BRCA1-linked and BRCA2-linked tumors can be used to classify sporadic tumors into “BRCA1-like” or “BRCA2-like” tumors, using the methods of the disclosure. A “non-BRCA-type” tumor is one that has a pattern of expression of ovarian cancer markers unlike a BRCA1-like or BRCA2-like tumor.

A vector is a nucleic acid molecule as introduced into a host cell, thereby producing a transformed host cell. A vector may include nucleic acid sequences that permit it to replicate in the host cell, such as an origin of replication, and may also include one or more therapeutic genes and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Unless otherwise explained, all technical and scientific terms used herein have the same meaning as commonly understood by one of ordinary skill in the art to which this invention belongs. The singular terms “a,” “an,” and “the” include plurals unless the context clearly indicates otherwise. Similarly, the word “or” is intended to include “and” unless the context clearly indicates otherwise. “Comprises” means “includes.” It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Description of Several Specific Embodiments

Provided herein are methods of diagnosing or prognosing development or progression of ovarian cancer in a subject, which methods involve detecting altered expression of at least one marker (e.g., a nucleic acid molecule such as one listed in Table 1 or genes, cDNAs or other polynucleotide molecules comprising one of the listed sequences, or a fragment thereof, or a protein, such as one encoded by such a nucleic acid molecule, or fragment of such protein). In certain embodiments, altered expression is detected in more than marker, for instance in at least 50, at least 100, at least 200, or at least 400 or more nucleic acid molecules listed in Table 1, or encoded for by a nucleic acid molecule listed in Table 1. In certain specific embodiments, no more than the molecules listed in Table 6, Table 7, Table 8, Table 9, Table 10 or Table 11 are included in such analysis.

Additionally provided herein are methods for the classification of ovarian tumors as BRCA1-like, BRCA2-like or non-BRCA-like tumors based upon expression profiles of selected markers. Using the expression profile data, multiple types of comparisons can be made to provide qualitative and quantitative information about the tumor type. Non-limiting examples of such comparisons include visual examination of color profiles of hierarchically clustered markers on a cDNA microarray, multidimensional scaling to the determine relative distance of the analyzed markers, and compound covariate prediction analysis to statistically classify a given tumor into one of two classes based upon the logarithmic expression ratio of the expression of at least one known classifying marker. In a specific non-limiting example, logarithmic expression ratios are generated and used to classify tumor types by comparing to markers known to have a logarithmic expression ratio associated with BRCA1-like, BRCA2-like or non-BRCA-like tumors (see Example 4).

Also encompassed herein are arrays containing two or more disclosed markers. Certain of such arrays are nucleic acid arrays that contain at least one marker, for instance at least one or more, such as 5, 10, 15, 25, 50, 100, 150, 200, 250, 300, 350, 400 or more nucleic acid molecules listed in Table 1 (or genes, cDNAs or other polynucleotide molecules comprising one of the listed sequences, or a fragment thereof), or a fragment of such protein, or an antibody specific to such a protein or protein fragment. Such arrays can also contain any particular subset of the nucleic acids (or corresponding molecules) listed in Tables 1-11 or all of those nucleic acids. Certain arrays (as well as the methods described herein) also may include nucleic acid molecules that are not listed in Table 1.

Certain of the encompassed methods involve measuring an amount of the ovarian cancer-related molecule in a sample (such as a serum or tissue sample) derived or taken from the subject, in which a difference (for instance, an increase or a decrease) in level of the ovarian cancer-related molecule relative to a standard such as a sample derived or taken from the subject at an earlier time, is diagnostic or prognostic for development or progression of ovarian cancer.

In some embodiments, altered expression of ovarian cancer-related nucleic acid molecules is detected using, for instance, in vitro nucleic acid amplification and/or nucleic acid hybridization. The results of such detection methods can be quantified, for instance by determining the amount of hybridization or the amount of amplification of the nucleic acid molecules.

Specific embodiments of methods for detecting altered expression of at least one ovarian cancer-related molecule use the arrays disclosed herein. Such arrays may be nucleotide (e.g., polynucleotide or cDNA) or protein (e.g., peptide, polypeptide, or antibody) arrays. In such methods, an array may be contacted with polynucleotides or polypeptides (respectively) from (or derived from) a sample from a subject. The amount and/or position of expression of the subject's polynucleotides or polypeptides then can be determined, for instance to produce a gene expression profile for that subject. Such gene expression profile can be compared to another gene expression profile, for instance a control gene expression profile from a subject having a known ovarian cancer-related condition. Similarly, protein arrays can give rise to protein expression profiles. Both protein and gene expression profiles can more generally be referred to as expression profiles. Expression profile data can be used to generate logarithmic expression ratios for use in compound covariate prediction analysis.

Other embodiments are methods that involve providing nucleic acids from the subject; semi-quantitatively amplifying the nucleic acids to form nucleic acid amplification products using primers; quantifying the amount of the nucleic acid amplification products; and comparing results to expression levels obtained using cDNA microanalysis. The sequence of such primers may be selected to bind specifically to a nucleic acid molecule listed in Table 1, or a nucleic acid molecule represented by those listed in Table 1. In specific examples of such methods, the primers are selected to amplify a nucleic acid product encoding topoisomerase II (TOP2A) (SEQ ID NO: 448), regulator of G-protein signaling 1 (RGS1) (SEQ ID NO: 398), invariant gamma-chain-associated protein (CD74) (SEQ ID NO: 89-91), epididymis-specific, whey-acidic protein (HE4) (SEQ ID NO: 60), major histocompatibility complex, class II, DR beta 1 protein (HLA-DRB1) (SEQ ID NO: 87-88), or zinc finger protein (ZFP36) (SEQ ID NO: 167-168).

Also encompassed are methods of ovarian cancer therapy, in which classification of a tumor of a patient into a BRCA1-like, BRCA2-like or non-BRCA-like tumor type aids in the selection of a treatment regimen. In some examples, the treatment selected is specific and tailored for the subject, based on the analysis of that subject's profile for one or more ovarian cancer-related molecules.

Other embodiments are kits for classifying tumors into a BRCA1-like, BRCA2-like or non-BRCA-like tumor class, which kits may include a binding molecule that selectively binds to the marker that is the target of the kit. In some examples of such kits where the marker is an ovarian cancer-related protein, the binding molecule provided in the kit may be an antibody or antibody fragment that selectively binds to the target ovarian marker protein. In other examples of such kits where the ovarian cancer-related marker level is a nucleic acid, the binding molecule provided in the kit may be an oligonucleotide capable of hybridizing to the nucleic acid marker molecule.

Further embodiments are methods of screening for a compound useful in treating, reducing, or preventing ovarian cancer or development or progression of ovarian cancer. Such methods involve determining if a test compound alters the gene expression profile of a subject (or cells of an in vitro assay) so that the profile more closely resembles a wild-type expression profile than it did prior to such treatment, and selecting a compound that so alters the gene expression profile. In specific examples of such methods, the test compound is applied to a test cell. In some of such methods, the profile is determined or measured in an array format.

Also encompassed are compounds selected using the methods described herein, which are useful in treating, reducing, or preventing ovarian cancer or development or progression of ovarian cancer.

Also disclosed herein are uses of identified target ovarian cancer-related molecules for the development of antibodies, including therapeutic antibodies that affect an ovarian cancer-related pathway. It is also envisioned that the disclosed ovarian cancer-related molecules can be used as vaccines, for instance as “cancer vaccines” to elicit an immune response from a subject that renders the subject more resistant to developing or progressing through a stage of ovarian cancer.

IV. Gene Expression Profiling of Ovarian Cancer Tumor Tissue Using Disclosed Markers

The present disclosure concerns gene expression profiling of ovarian tumor tissue from a subject for use in diagnosing, prognosing, staging, preventing, and treating the disease. Measurement of expression of genes within a tissue sample provides information regarding proteins that may be active during cancer mechanisms. Hence, the gene expression profile of tumor tissue may be compared against the profile for known markers for ovarian cancer, such as those disclosed herein (see Table 1).

Using the gene expression profile, an ovarian tumor from a subject may be classified into a BRCA1-like, BRCA2-like, or non-BRCA-like tumor. Because the prognosis for a patient having a BRCA1 or BRCA2 mutation is poorer than for patient having non-BRCA-like mutations, classification of tumors into these groups is helpful in selecting treatment strategies and aids a clinician in deciding whether to employ a more aggressive regimen in treating the patient, for instance radiotherapy, chemotherapy, or surgical removal of the affected tissue. In addition, classification of a sporadic tumor into a BRCA1-like or BRCA2-like classification may provide similar guidance in treating the patient. For example, a subject who has a BRCA1-like or BRCA2-like sporadic tumor may be treated similarly to a subject who has a BRCA1-linked or BRCA2-linked tumor. The identification of BRCA1- or BRCA2-like sporadic tumors also allows tumors (or subjects) to be selected for specific drug regimens that are particularly effective with the associated mutation type.

The ovarian cancer-linked markers disclosed herein are believed to be useful as diagnostic or prognostic indicators of BRCA1-like, BRCA2-like and non-BRCA-like ovarian cancer. In addition, the markers are believed to be useful in applications for treating ovarian cancer as the basis of new therapeutic targets, for the development of new anti-cancer therapeutic compounds, and/or to select particularly appropriate existing treatments. For example, the expression levels of these markers can be examined to monitor the effectiveness of anti-cancer treatments where an increase in or decreased level of nucleic acid expression opposite of the ovarian cancer-indicative pattern disclosed herein indicates an effective anti-cancer treatment. Beyond use in generating an expression profile, certain of the identified genes or EST sequences provided herein arc believed to have individual use as cancer markers.

A. Generating Gene Expression Information and Logarithmic Expression Ratios.

cDNA microanalysis allows for simultaneous analysis of the expression of multiple genes within various tissue samples, and is therefore useful in generating gene expression profiles. To perform cDNA microarray analysis, RNA is isolated from a subject and cDNA is synthesized from the RNA according to standard methods (see Sambrook et al., Molecular cloning, a laboratory manual. 2^{nd} ed. Cold Spring Harbor Laboratory, Cold spring Harbor, N.Y., 1989). Relative over-expression of the mRNA in the cancerous tissues can be measured against non-cancerous reference baselines (e.g., ovarian tissue from a subject not having ovarian cancer or an ovarian cell line, such as an immortalized ovarian cell line), to provide a framework for determining normal expression versus altered expression (genes that are either overexpressed or underexpressed). Nucleic acids that are overexpressed may be used as markers for ovarian cancer, while genes that are underexpressed may be putative tumor suppressors.

cDNA microarrays containing 7,651 sequence-verified features were constructed and applied to analyze the mRNA expression profile of sixty-one subjects with pathologically-confirmed epithelial ovarian adenocarcinoma having matched clinicopathologic features (see Alizadeh et al., Nature 403: 503-511, 2000; Perou et al., Nature 406: 747-752, 2000; Bubendorf et al., J. Natl. Cancer Inst., 91(20): 1758-64, 1999; Welsh et al., Proc Natl Acad Sci. USA 98: 1176-1181, 2001). These included eighteen cases linked to BRCA1 founder mutations, sixteen cases linked to BRCA2 founder mutations, and 27 cases negative for any founder mutations (e.g., sporadic ovarian epithelial cancer). These samples were compared to expression levels of these same features in an immortalized normal ovarian surface epithelium cell line (IOSE). Statistical tools, including a modified F-test with P<0.0001 considered significant (e.g., a 99.99% confidence level) were then used to analyze the data (e.g., to differentiate gene expression profiles associated with ovarian cancer), enabling a comprehensive, genomics-based analysis of the mRNA expression profiles of these ovarian cancer subjects.

The logarithmic expression ratios for the spots on each array were normalized by subtracting the median log ratio for the same array. Data were filtered to exclude spots with size less than 25 μm, intensity less than two times background or less than 300 units in both red and green channels, and any flagged or missing spots. In addition, any features found to be missing or flagged in greater than 10% of the arrays were not included in the analysis. Application of these filters resulted in the inclusion of 6,445 of the total 7,651 features in subsequent analyses. Statistical comparison between tumors groups was performed using the “BRB Array Tools” software (developed by Dr. Richard Simon and Amy Peng, Biometrics Research Branch, Division of Cancer Treatment and Diagnosis, NCI, USA), consisting of a modified F-test with P<0.0001 (99.99% confidence level) considered significant (see Example 4). This stringent P value is selected in lieu of the Bonferroni correction for multiple comparisons, which was deemed excessively restrictive (see Bland and Altman, B.M.J.: 310: 170, 1995). See Example 4 for an example of how an ovarian tumor is analyzed using the disclosed methods.

In addition to statistical analysis, multidimensional scaling (MDS) and hierarchical clustering techniques using a correlation metric and average linkage were used for evaluating overall gene expression. Using these techniques, a large set of genes and other encoding sequences (e.g., expressed sequence tags, ESTs) have been identified (Table 1), the expression of which varies in subjects having ovarian cancer (see Addendum). Other confidence levels could be used to select ovarian cancer-related molecules, such as 98%, 95%, 90%, 85%, and so forth (see Jain et al., IEEE Transacations on Pattern Analysis and Machine Intelligence 22(1): 4-37, 2000). Molecules identified as being linked to ovarian cancer (referred to generally herein as ovarian cancer-related molecules) using the methods described herein can be arranged on arrays for use in diagnostic and prognostic methods. Specific arrays are contemplated that are constructed using molecules identified at differing confidence levels. Specific examples of such arrays include arrays that detect altered expression of at least 2, 5, 10, 20, 30, or 50 of these molecules.

B. Comparison of Ovarian Epithelial Adenocarcinoma Cells to Immortalized Ovarian Surface Epithelium Cells

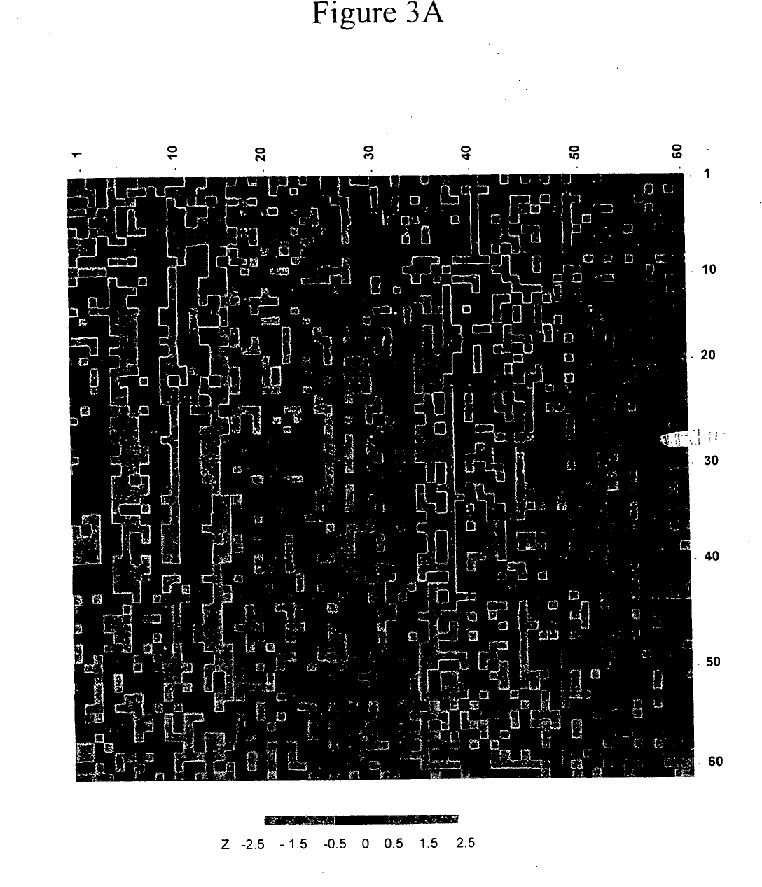

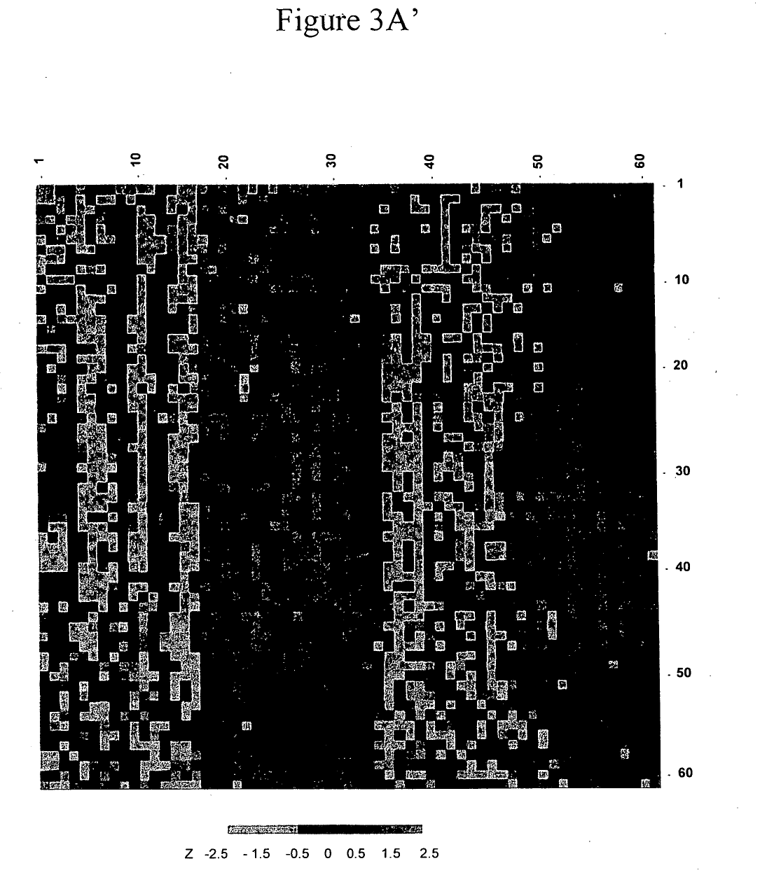

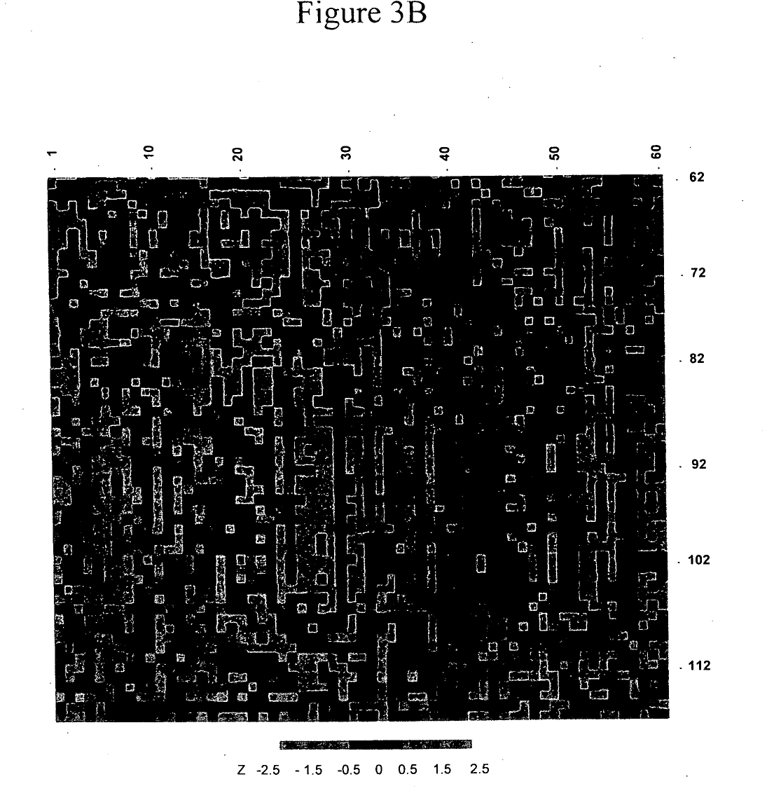

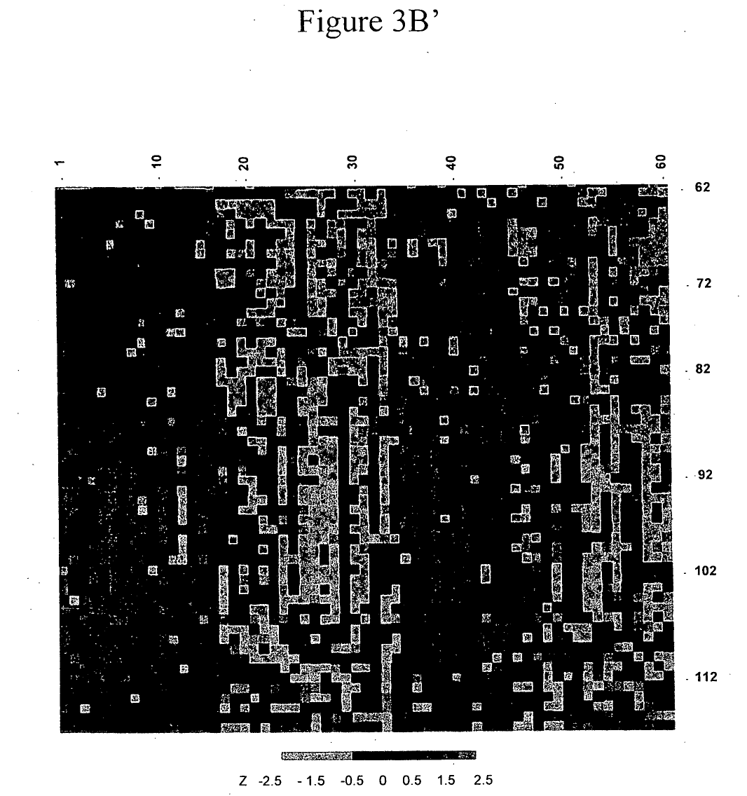

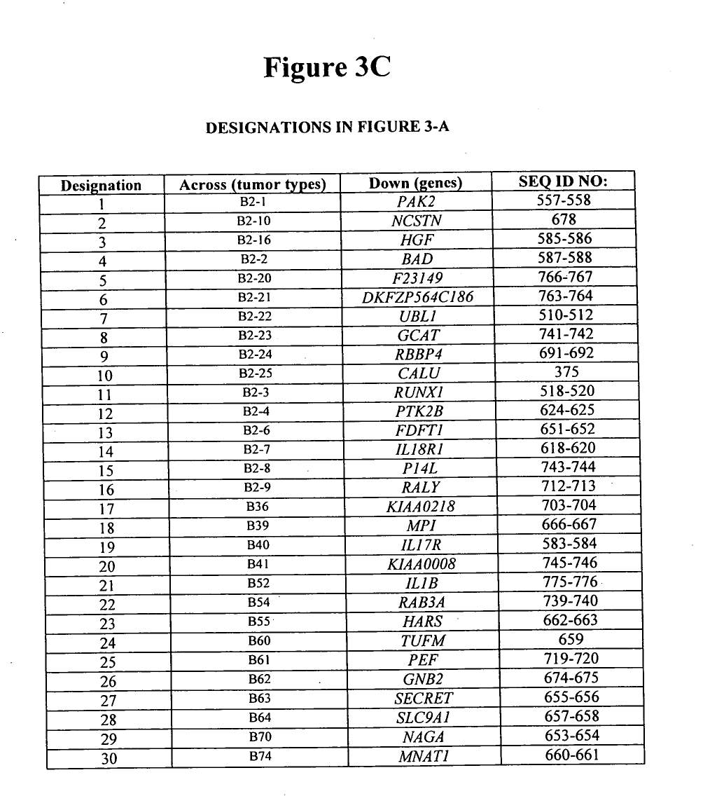

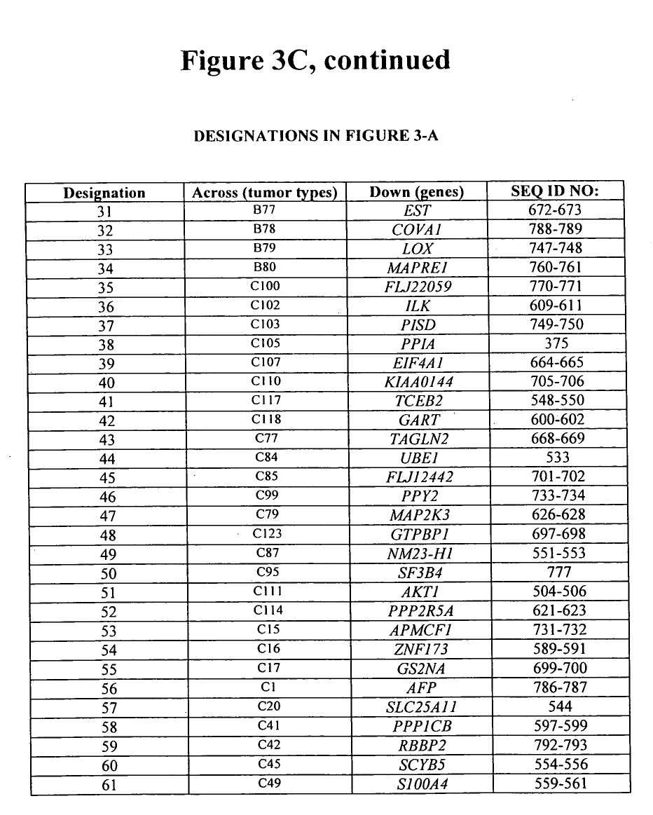

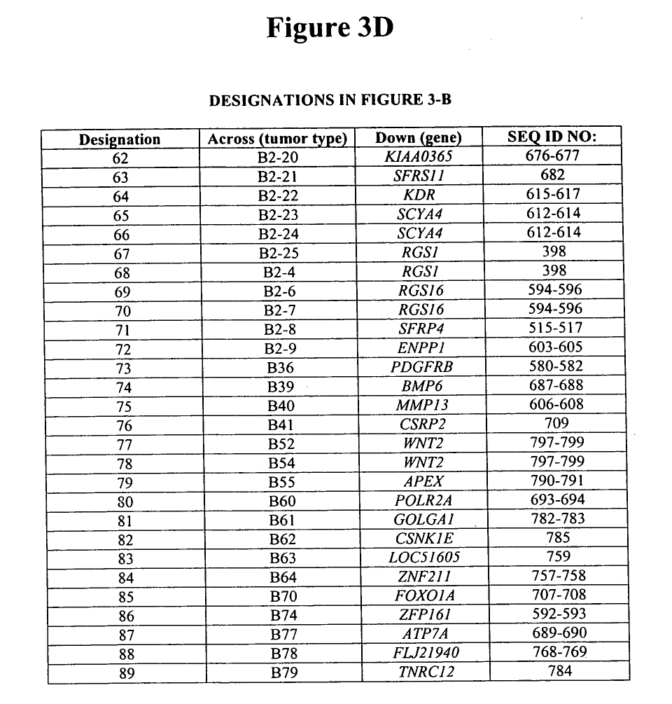

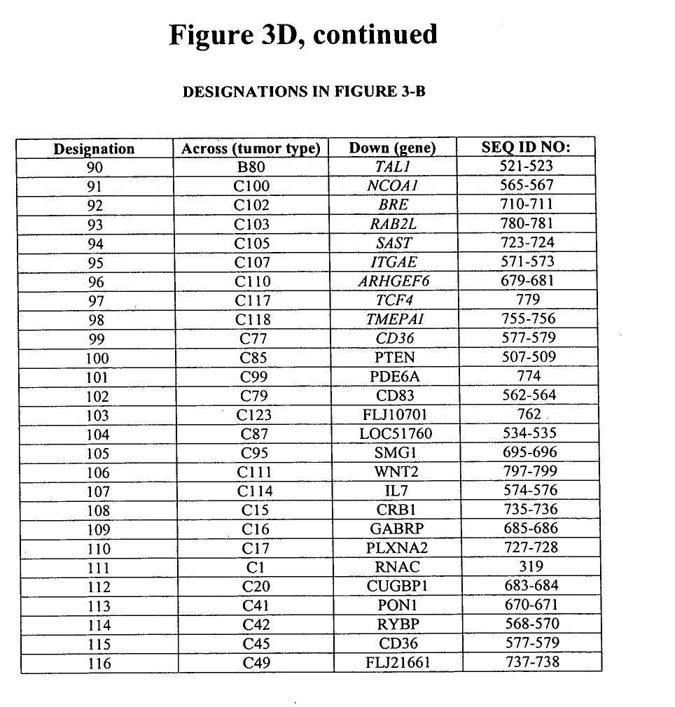

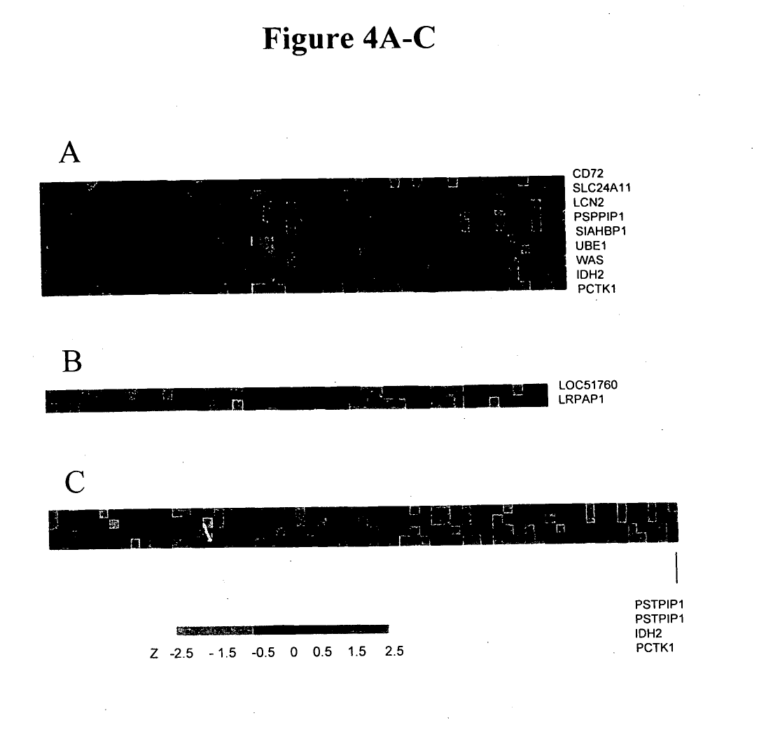

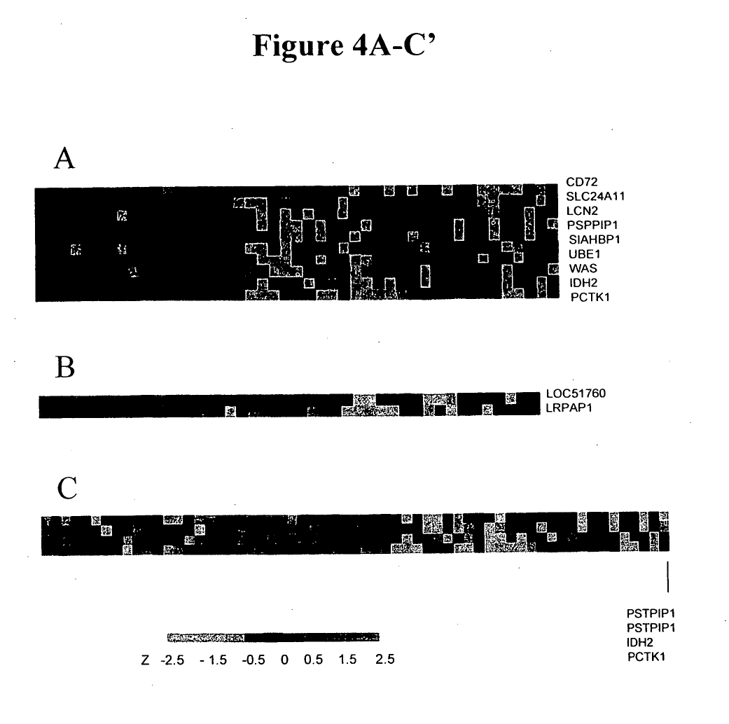

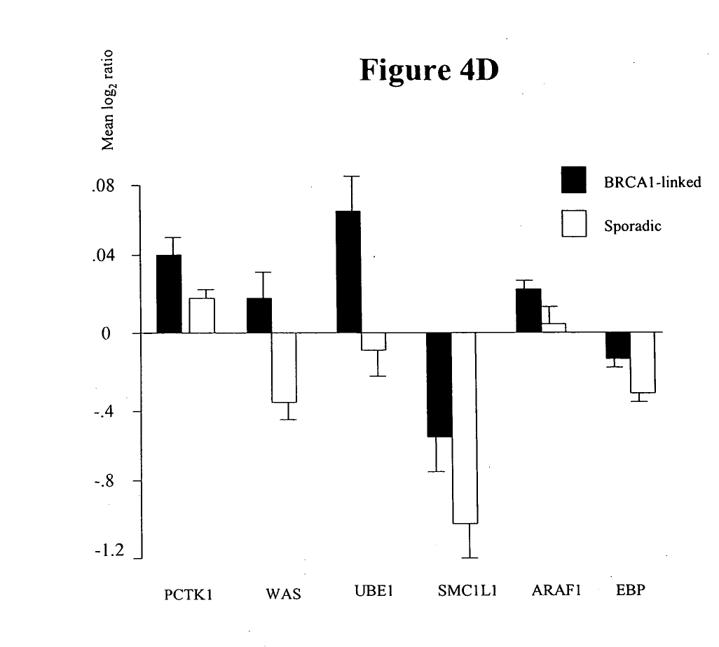

In a comparison of ovarian epithelial adenocarcinoma cells to immortalized ovarian surface epithelium cells, the largest contrast in gene expression was observed between BRCA1- and BRCA2-linked tumors, with multiple genes showing significant differences in expression levels. This group of genes was also able to segregate the sporadic tumors into two major “BRCA1-like” and “BRCA2-like” subgroups, indicating that BRCA-related pathways are also involved in sporadic ovarian cancers. In addition, two previously unreported gene expression patterns were noted. First, six of the genes differentially expressed between BRCA1-linked and sporadic tumors map to Xp11.23 and all exhibited higher mean expression levels in the BRCA1-linked samples [WAS (SEQ ID NO: 524-526), EBP (SEQ ID NO: 529), SMC1L1 (SEQ ID NO: 529), PCTK1 (SEQ ID NO: 527-528), ARAF1 (SEQ ID NO: 531-532), and UBE1 (SEQ ID NO: 533), see FIG. 3]. Second, compared to immortalized ovarian surface epithelium cells, several interferon-inducible genes were noted to be overexpressed in the majority of all tumor samples [SIAT1 (SEQ ID NO: 73), TNFSF10 (SEQ ID NO: 104-106), ABCB1 (SEQ ID NO: 164-166), CP (SEQ ID NO: 83-84), HLA-DRB5 (SEQ ID NO: 85-86), HLA-DRB1 (SEQ ID NOS: 87-88, 100, 101-103), CD74 (SEQ ID NO: 92-93), HLA-DRA (SEQ ID NO: 94-96), HLA-DPA (SEQ ID NO: 97-99), IFITM1 (SEQ ID NOS: 50-51, 52-54), IFITM2 (55-57, 58-59), A2M (SEQ ID NO: 193-195), G1P3 (68-69), 1GKC (SEQ ID NOS: 112-114, 115-116), SCYB10 (SEQ ID NO: 120-121), Col3A1 (SEQ ID NO: 141-143), HLA-B (SEQ ID NO: 154-156), and HLA-C (SEQ ID NO: 157-159), see FIG. 4]. In terms of overall differential gene expression, BRCA1 and BRCA2-linked tumors express genes more different from each other than from sporadic (non-BRCA-linked) tumor samples.

The identified ovarian cancer-related genes represent putative mediators of ovarian cancer, and as such are candidate targets for the development of novel therapeutics for the treatment of ovarian cancer using conventional techniques. By way of example, a candidate drug, targeted at restoring expression of a gene of the disclosure, could be examined using cDNA microarray analysis for utility in influencing growth of ovarian cancer cells. Thus, use of cDNA microarray techniques for genomics-based discovery of genes variably expressed during ovarian cancer provides for the identification of novel therapeutic targets for treatment of ovarian cancer.

It is contemplated that certain of the ovarian cancer markers identified herein encode or correspond to soluble proteins, while others encode or correspond to membrane associated or membrane integral proteins, some of which are exposed at least to a certain extent on the exterior of a cell in which they are expressed. In some embodiments, those ovarian cancer-related molecules that are expressed at or on the surface of a cell are selected as therapeutic targets, for instance for targeting with an antibody-based therapy, which is facilitated by the access of the ovarian cancer-related molecule to the extracellular matrix. These ovarian cancer markers may be described as being “drug accessible.” In addition, such soluble ovarian cancer markers, if secreted, may be detected in a blood or serum sample from the subject.

C. Comparison of Ovarian Epithelial Cancer Cells to Normal Postmenopausal Ovarian Samples.

cDNA microarrays containing 7,600 sequence-verified features were constructed and applied to analyze the mRNA expression profile of 61 subjects with ovarian epithelial cancer as compared to two normal postmenopausal ovarian samples.

Gene expression in each sample (normal or tumor) was directly compared to a “reference RNA” consisting of a mix of nine different human cell lines (breast adenocarcinoma, hepatoblastoma, cervical adenocarcinoma, testicular embryonal carcinoma, glioblastoma, melanoma, liposarcoma, histiocytic lymphoma, T cell lymphoblastic leukemia, and plasmacytoma/myeloma, Stratagene, La Jolla, Calif.). The raw gene expression data was used to calculate the logarithmic expression ratio for each gene. The logarithmic expression ratios (“log ratio”) obtained from this comparison were then normalized and statistically compared to one another, providing for indirect comparison of gene expression in tumors and normal ovarian samples. This was accomplished by scoring the magnitude of differential expression of each gene (between normal and cancer samples) according to the formula:

$$\frac{\begin{matrix}( average cancer log ratio - \\ average normal log ratio )\end{matrix}}{\begin{matrix}( standard deviation cancer + \\ standard deviation normal )\end{matrix}} = \begin{matrix}magnitude of differential \\ expression\end{matrix}$$

In genes showing a large mean expression difference between normal and cancerous samples, the magnitude of differential expression has a greater value, while the intra-group variability in expression ratios is low.

Genes were then ranked according to the magnitude of differential expression and the highest-ranking genes were considered to be the best candidates for differentiating normal from malignant ovarian samples (see Furey et al., Bioinformatics 16(10): 906-14, 2000).

Using these techniques, a large set of genes and other encoding sequences (e.g., ESTs) that are under-expressed in subjects having ovarian cancer have been identified (see Table 4). These under-expressed ovarian cancer markers represent putative tumor suppressors, and as such are candidate targets for the development of novel therapeutics for the treatment of ovarian cancer using conventional techniques. By way of example, induction of expression of one or more of these markers through therapeutic means (e.g., induction by a drug or gene therapy) may inhibit tumor growth and/or increase tumor cell death, for instance through stimulation of apoptotic pathways.

Furthermore, a large set of genes and other encoding sequences (e.g., ESTs) have been identified (see Table 5), the expression of which is overexpressed in subjects having ovarian cancer. These overexpressed ovarian cancer markers represent putative mediators of ovarian cancer, and as such are candidate targets for the development of novel therapeutics for the treatment of ovarian cancer using conventional techniques. Over-expression of one or more such markers can also be detected in the body (for example using a serum test to detect or monitor progression of ovarian cancer).

In addition, six of the markers identified herein (e.g., WAS (SEQ ID NO: 524-526), PCTK1 (SEQ ID NO: 527-528), UBE1 (SEQ ID NO: 533), SMC1L1 (SEQ ID NO: 529), ARAF1 (SEQ ID NO: 531-532), and EBP (SEQ ID NO: 529)) have all been mapped to chromosome Xp11 (see Example 1). Hence, this chromosome could contain additional genes and ESTs that may be useful as markers for prognosing, diagnosing and monitoring ovarian cancer. The methods of the disclosure can be used to find additional genes and ESTs in this region for use as ovarian cancer markers.

V. Methods of Classifying Tumors into Subgroups

Disclosed herein are multiple methods of classifying tumors into subtypes based upon the expression of disclosed ovarian tumor markers (see Table 1).

A. Comparison of Raw Expression Data

The expression data of one or more ovarian cancer markers can be compared between samples and analyzed to detect differences in expression between the markers. The expression of an individual marker can be stated in ratio or “fold” form relative to the expression of the standard. For instance, in Table 4, the average logarithmic ratio of the gene expression for the standard (“normal”) for ITM2A (SEQ ID NO: 202) is 1.145, while the average logarithmic ratio of the gene expression in cancer cells was −2.036. These numbers can be compared to derive a value for the difference in expression by calculating the expression ratio of each number, and dividing the expression ratio for the average log cancer value by the expression ratio for the average log normal value. Hence:
Expression ratio of average log normal: 2^{1.145}=2.211
Expression ratio of average log cancer 2^{-2.036}=0.244
Ratio (cancer to normal)=(0.244)/(2.211)=0.110

Thus, ITM2A is under-expressed in cancer by a ratio of 0.110 to 1 (i.e., in ovarian cancer tissue, ITM2A expresses at approximately 10% of the expression level seen in wild-type cells).

Collections of such data can be assembled to provide a gene expression profile, as discussed above. With such profiles, the standard deviation of the expression ratio of each gene can be measured by obtaining the square root of the variance of the expression data as described by Jaccard and Becker (in Statistics for the Behavioral Sciences, 2^{nd} ed., Wadsworth Publishing Co., Belmont, Calif., 1990) and Myers and Well. (in Research Design and Statistical Analysis, University of Massachusetts, Amherst, Mass., 1995).

Further analysis can include a Student's t-test, to determine if the mean expression of two groups (e.g., BRCA1-like and non-BRCA-like, BRCA2-like and non-BRCA-like, etc.) are statistically different from each other.

Due to the range over which genes may express, it may be useful to perform statistical analyses using the logarithmic expression value for each marker (see Example 1). However, calculations using the logarithmic expression values may dilute the ability of certain analyses to determine differences. Hence, it may be useful to employ multiple methods of analysis to ascertain relative values in expression (see Jain et al., IEEE Transacations on Pattern Analysis and Machine Intelligence 22(1): 4-37, 2000).

B. Visual Analysis of Hierarchical Clustering

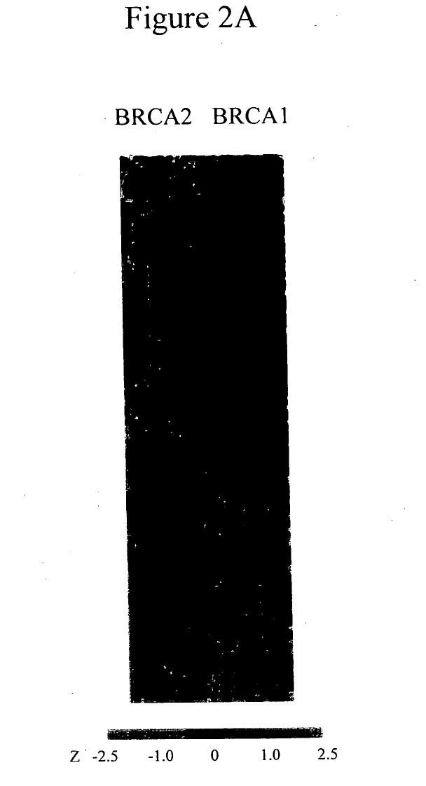

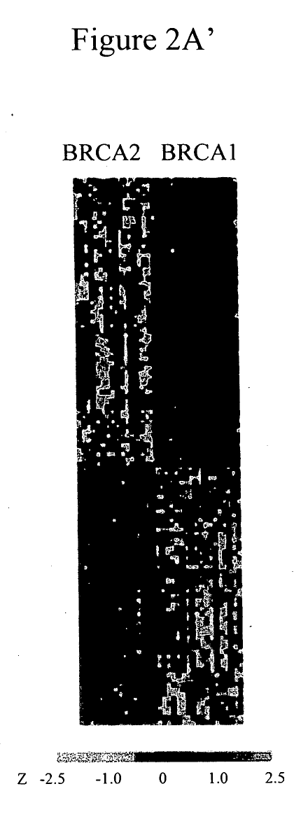

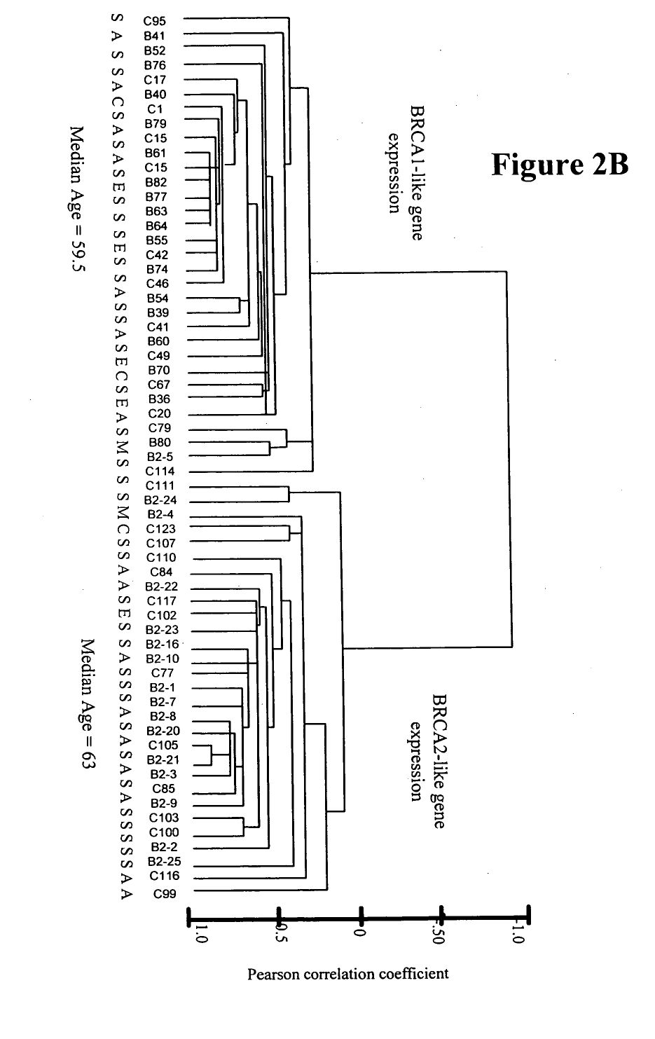

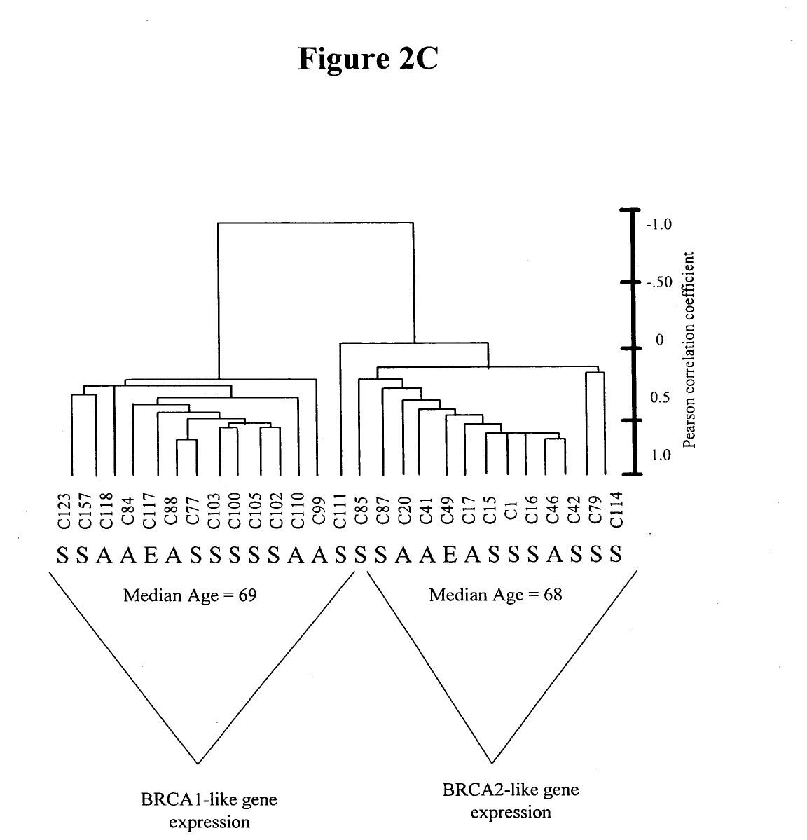

Methods disclosed herein include hierarchical clustering analysis of genes with statistically significant differential expression between sets of tumor groups. Hierarchical clustering can be used to cluster objects (e.g., genes, such as the ovarian cancer markers listed in Table 1) to represent relationships among the objects. The relationships are represented, for example by a tree whose branch lengths reflect the degree of similarity between the objects (see e.g., FIG. 2B).

Optionally, hierarchical clustering can be combined with a graphical representation of the primary data by representing each data point with a color that quantitatively and qualitatively reflects the original experimental observations. The use of color representations, along with statistical organization, provides a graphical display that provides visual information about expression of the genes. Hence, the methods disclosed herein can provide visual information regarding degrees of similarity (e.g., patterns of under-expression or over-expression) between assessed genes in different samples, for instance in samples of BRCA1-linked, BRCA2-linked and sporadic ovarian tumor samples (see FIG. 2B).

At the first iteration, each object is considered to be its own group, and the pair of objects with the smallest distance between them is merged into a new group. Each subsequent iteration merges two groups to form a new group, until finally all objects end up merged into a single group. The classification tree, or dendrogram, graphically represents the sequence of clusters formed at each iteration of merges, as well as the distance between clusters at each merge (here, FIG. 2). This technique is widely employed to represent gene expression information obtained from microarray experiments (see Eisen et al., Proc. Natl. Acad. Sci. U.S.A. 95(25): 14863-8, 1998).

The gene expression data disclosed herein were analyzed by calculating the Pearson correlation coefficient to obtain a gene expression similarity metric. To describe, Gi is (log-transformed) primary gene expression data for gene G in each tumor sample, represented as variable i. For any two genes X and Y observed over a series of N tumor samples, a similarity score can be computed as follows:

$$S ( X , Y ) = \frac{1}{N} {\sum }_{i = 1 , N} ( \frac{{X}_{i} - {X}_{offset}}{{Φ}_{X}} ) ( \frac{{Y}_{i} - {Y}_{offset}}{{Φ}_{Y}} )$$

$$where$$

$${Φ}_{G} = \overline{{\sum }_{i - 1 , N} \frac{{( {G}_{i} - {G}_{offset} )}^{2}}{N}} .$$

When G_{offset} is set to the mean of the gene expression levels of the tumor samples for gene G, then Φ_{G} becomes the standard deviation of G, and S(X, Y) is exactly equal to the Pearson correlation coefficient of the gene expression levels for genes X and Y. Values of G_{offset} that are not the average of the gene expression levels for gene G are used when there is an assumed unchanged or reference state (e.g., the gene is not over-expressed or under-expressed) represented by the value of G_{offset}, against which changes are to be analyzed; in all of the examples presented here, G_{offset} is set to 0, corresponding to a fluorescence ratio of 1.0.

By way of example, FIGS. 2A and 2A′ demonstrate that expression of the disclosed markers can be used to visualize different tumor types. Hierarchical clustering was performed using with a Pearson correlation metric and average linkage were used for evaluating overall gene expression for the sixty-one BRCA1-linked, BRCA2-linked and sporadic tumors (see Example 1). When applicable, all statistical tests were two-sided.

In FIG. 2, B2 represents BRCA2-linked tumors, and B1 represents BRCA1-linked tumors. The red and green intensities represent standard normal deviation (Z score) values from each marker's means expression level (represented as black) across the sixty-one tumors samples. Red represents increased expression and green represents decreased expression. The differences in gene expression can be appreciated by looking at the groupings apparent in FIG. 2A. The genes in the left half of the FIG. 2A are from BRCA2-linked tumors and the genes in the right half are from BRCA1-linked tumors. As can be seen with casual observation, gene expression between these two tumor groups differs relative to the control (IOSE cells). Specifically, BRCA2-linked tumors contain under-expressing genes that correlate to these genes in the upper left and lower right quadrants of FIG. 2A, which are represented as primarily green in color. Furthermore, the genes in the upper right and lower left quadrant, which are represented as primarily red in color, correlate to genes that are generally over-expressed relative to the control IOSE cells. Hence, hierarchical clustering can be used to qualitatively visualize differences in the expression patterns of samples.

C. Multidimensional Scaling

Multidimensional scaling is a dimension reduction procedure that can be used for visualization purposes. Each experiment can be represented by its expression profile, which is a K-dimensional vector of log-ratios, where K is the number of clones represented after filtering. The multidimensional scaling procedure reduces each experiment's expression profile from K-dimensional space to 3-dimensional space, by attempting to preserve distances between the N experiment vectors. The distance metric needs to be specified when using the multidimensional scaling tool. First, the N×N distance matrix is computed, which quantifies the relationships between the N experiments in the series of chips. For each of the N vectors in K-dimensional space, the multidimensional scaling procedure finds a vector in 3-dimensional space, such that the N×N distance matrix computed in 3-dimensional space approximates the N×N distance matrix computed in K-dimensional space. The relationships between the N experiments can then be visualized by plotting the N vectors in 3-dimensional space, in which each of the N points represents a single experiment. A rotating 3-dimensional visualization tool can be used for discovery of experiment clusters.

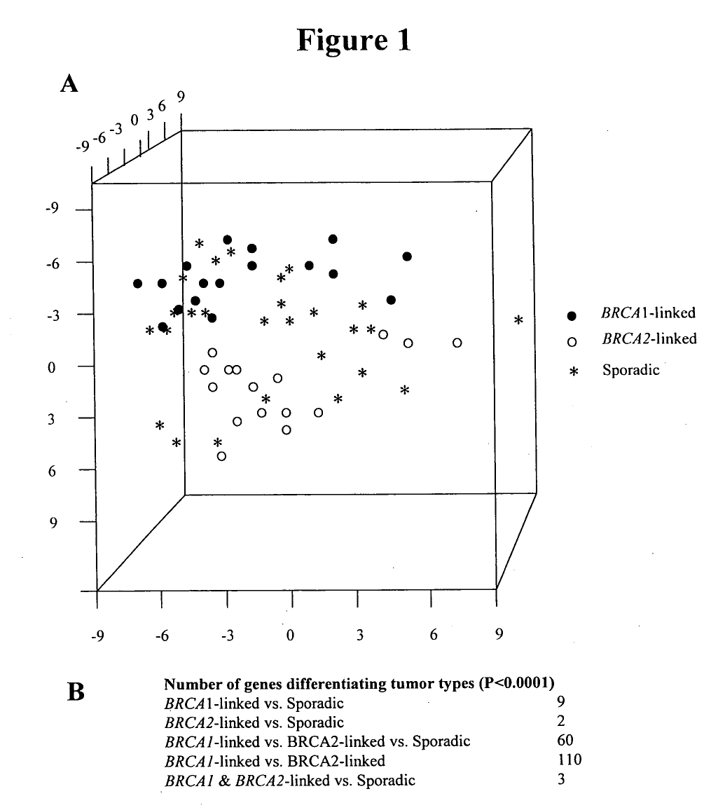

By way of example, the gene expression data of 6445 filtered genetic elements of the sixty-one ovarian tumor samples (see Example 1) was used in multidimensional scaling to generate a 3-D diagram for visualization of the respective differences between the expression patterns of each tumor sample. As seen in FIG. 1, the data segregate into different areas of the 3-D space based on similarities in gene expression within the tumor type. In particular, the BRCA1-linked tumors (dark circles) segregate higher into the cube than the BRCA2-linked tumors (open circles). The sporadic tumor samples (asterisks) also fell into higher and lower areas of the cube, indicating that they segregate into BRCA1-type and BRCA2-type expression patterns. Thus, multidimensional scaling can be used to make a qualitative distinction regarding the expression patterns of these samples.

Multidimensional scaling can be used to qualitatively assess the expression pattern of an unknown tumor type. Expression data for a plurality of BRCA1-type and BRCA2-type markers is generated using the tumor tissue (for instance, on a cDNA microarray) relative to a standard ovarian tissue (e.g., from a subject not having ovarian cancer, immortalized ovarian epithelial cells, etc.), and logarithmic ratios of the gene expression data are calculated. To compare the pattern of expression of the plurality of the known BRCA1-type and BRCA2-type markers to the unknown ovarian sample, the K-dimensional vectors of the logarithmic expression ratios for all expression data are calculated as discussed above. Next, the K-dimensional vectors are plotted in a 3-dimensional space and the layout of the data compared. Similar to FIG. 1, the unknown sample data should cluster either near the BRCA1-like or BRCA2-like tumors, or alone (which would indicate that it is a non-BRCA-like tumor). Hence, multidimensional scaling can be used to make a qualitative distinction regarding the expression patterns of an unknown samples in comparison to known BRCA1-type and BRCA2-type markers. In addition, more than one unknown sample can be used in this analysis.

D. Compound Covariate Predictor Analysis

Segregation into tumor types can be performed using compound covariate predictor analysis, which creates a multivariate predictor for one of two classes to each sample (see Example 4). Markers included in the multivariate predictor are those that are univariately significant at the selected significance cutoff (e.g., P<0.0005). The multivariate predictor is a weighted linear combination of log-ratios (or log intensities for single-channel arrays) for genes that are univariately significant. The weight consists of the univariate t-statistics for comparing the classes, and is calculated using the equation:
CCP=Φᵢtᵢ*(xᵢ−mᵢ)
where tᵢ=t-value for gene i (see Table 10), xᵢ=logarithmic ratio of the gene expression (i) in the new sample to be classified, and mᵢ=midpoint between the two classes for gene i (see Table 10). The index i runs over all the genes that are significant in the original analysis (i.e. all 62 genes in Table 10). If the log ratio xᵢ is missing for gene i in the new sample to be classified, then it should be assigned as mᵢ for that gene, to cause the result of the calculation to be zero for that gene. If the compound covariate predictor value is positive, then the tumor classified as one of the first type (e.g., BRCA1-like). If the compound covariate predictor value is negative, then the tumor is classified as belonging to the second type (e.g., BRCA2-like).

A second method of tumor classification using a compound covariate predictor model can be found in Radmacher et al., “A paradigm for class prediction using gene expression profiles,” found on the National Cancer Institute Internet website. This publication is expressly incorporated by reference herein.

In order to determine whether a tumor is classified as BRCA1-like or BRCA2-like using a single markers in Table 10, the following steps are used:
1. Gene expression information is obtained, for instance on a cDNA microarray, using the same standard (e.g., IOSE cells) that was used to obtain the marker data.
2. The gene expression data is converted into a logarithmic ratio using log base 10. Hence, a tumor that has a gene expression value for gene KIAA00008 of 0.45 would have a log base 10 ratio of −0.346.
3. The midpoint value of Table 10 is subtracted from the logarithmic ratio, and multiplied by the t-value for KIAA00008 for the data set. Thus,
   [(−0.346)−(−0.431)]*(−8.0421)=−0.51.

The values for the average logarithmic ratio for BRCA1-linked and BRCA2-linked values in the data set are then consulted. The obtained value will fall between the midpoint and one of these values because genes in which larger values of the logarithmic ratio are assigned to one class (e.g., BRCA1-linked) will have weights of a value that is more negative with respect to the midpoint value (e.g., −0.56864), whereas genes in which larger values of the logarithmic ratios are assigned to the other class (e.g., BRCA2-linked) will have weights of a value that is more positive with respect to the midpoint value (e.g., −0.29414). Hence, the obtained value, 0.1930, would fall on the more negative side of this data, and would therefore be classified as a BRCA2-like data set.

If this same analysis is performed using multiple markers, the method remains the same except that the data can be summed prior to performing the analysis. This method is a multivariate approach of the compound covariate analysis, and can be used to determine whether the pattern of expression of an unknown tumor is similar to a BRCA1-like or BRCA2-like pattern of expression.

Further analysis, such as a “leave-one-out” approach may additionally be employed to test the ability of the Compound Covariate Predictor to classify the tumors into additional subtypes, such as resistance to a therapeutic compound. See Radmacher et al., “A paradigm for class prediction using gene expression profiles,” found on the National Cancer Institute Internet website.

E. Comparisons Using Databases

Due to the large amount of information associated with the analysis methods disclosed herein, it may be particularly useful to construct and/or consult databases of information for use in the analysis.

By way of example, the information generated by the methods of the disclosure can be stored in databases, such as a database of a plurality of markers known to express differently in BRCA1-like and BRCA2-like tumors (e.g., Table 9). Such databases may be made publicly available, such as the Stanford Microarray Database (maintained by Stanford University, see Sherlock et al., Nucleic Acids Res., 29(1): 152-155, 2001). These databases may be used to store reference data for use with the classification methods of the disclosure. In addition, such databases can be used to provide information regarding markers of potential use in diagnosing, prognosing, or monitoring ovarian cancer, for use by clinicians.

The use of databases to search for stored information is disclosed in U.S. Pat. Nos. 5,871,697 and 6,519,583 the methods of which are expressly incorporated herein.

VI. Kits for Measuring the Level or Function of Ovarian Cancer-Related Molecules.

The nucleic acid sequences and ESTs disclosed herein can be supplied in the form of a kit for use in detection or monitoring ovarian cancer. In such a kit, one or more of the nucleic acid sequences and/or ESTs in Table 1 are provided in one or more containers, or in the form of a microarray. The kit may also contain reagents for use in preparing a biological sample of a subject for screening with the kit. The container(s) in which the reagent(s) and microarray(s) are supplied can be any conventional container that is capable of holding the supplied form, for instance, plastic boxes, microfuge tubes, ampoules, or bottles. In some applications, negative controls obtained from a subject free from ovarian cancer may be provided in pre-measured (e.g., single use) amounts in individual, typically disposable, tubes or equivalent containers. With such an arrangement, the sample to be tested for the presence of ovarian cancer can be added to the testing container and tested directly.

The amount/number of each testing reagent and container supplied in the kit can be any appropriate amount, depending for instance on the market to which the product is directed. For instance, if the kit is adapted for research or clinical use, the amount of each testing reagent and container provided would likely be an amount sufficient to screen several biological samples. Those of ordinary skill in the art know the amount of testing reagent that is appropriate for use in a single container. General guidelines may for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al. (In Current Protocols in Molecular Biology, Greene Publ. Assoc. and Wiley-Intersciences, 1992).

A kit may include more than two nucleic acid sequences or ESTs, in order to facilitate screening of a larger number of ovarian cancer markers or tumor suppressors. For instance, the sequences set forth in Table 1, or a subset of (e.g., 5, 10, 15, 20, 50, 100, 150, 200, 250, 300, 350, 400 or more) of these sequences, may be provided. By way of example, a provided subset could include the markers set forth in Table 6, Table 7, Table 8, Table 9, or Table 10. These sets of sequences are provided by way of example only, and are not intended to be limiting examples.

In some embodiments of the current disclosure, kits may also include the reagents necessary to carry out screening reactions, including, for instance, RNA sample preparation reagents, appropriate buffers (e.g., polymerase buffer), salts (e.g., magnesium chloride), and secondary detection reagents (e.g., cyanine 5-conjugated dUTP).

Kits may in addition include either labeled or unlabeled sequences for use in detection of the expression levels.

Embodiments of the disclosure are illustrated by the following non-limiting Examples.

### EXAMPLE 1

### Identification of Genes with Altered Expression in Ovarian Cancer

This example describes how a first subset of the disclosed ovarian cancer-related nucleic acid molecules were identified. These ovarian cancer-related molecules show differences in expression in subjects having ovarian cancer compared to normal ovarian surface epithelial cells and are classified according to their BRCA-1, BRCA-2, and sporadic tumor status. The results of these studies have been published in Jazaeri et al., J. Natl. Cancer Inst., 94(13): 990-1000, 2002, which is incorporated by reference in its entirety herein.

Methods and Material:

Clinicopathologic characteristics of BRCA-linked and sporadic ovarian cancers: Sixty-one cases of pathologically-confirmed epithelial ovarian adenocarcinoma from the Memorial Sloan-Kettering Cancer Center were studied and screened for founder mutations. These included eighteen cases linked to BRCA1, sixteen cases linked to BRCA2, and twenty-seven sporadic cases. All patients were self-identified as Ashkenazi Jews and after informed consent underwent genotyping for germ-line founder mutations in BRCA1 (185delAG and 5382insC) and BRCA2 (6174delT) (see Boyd et al., JAMA: 283: 2260-2265, 2000). Those cases with a BRCA mutation were categorized as having hereditary ovarian cancer and those without such a mutation as having sporadic ovarian cancer.

Tumor samples: In order to minimize confounding variables, BRCA1-linked, BRCA2-linked, and sporadic tumors of similar stage, grade, and histology were selected from the sixty-one individuals studied [18 BRCA1 (185delAG, 5382insC), 16 BRCA2 (6174delT), 27 sporadic tumors). The majority of tumors in all three groups were characterized by advanced stage, moderate to high grade (grade 2 or 3), and a predominance of serous histology. Hence, the clinicopathologic parameters of selected samples were well-matched and in agreement with those reported previously for these tumors types (see Boyd et al., JAMA: 283: 2260-2265, 2000: Ramus et al., Genes Chrom. Cancer: 25: 91-96, 1999).

All tumor samples had been flash frozen, embedded in OCT medium, and stored at −80° C. Isolation of RNA was performed using the RNeasy columns (Qiagen, Valencia, Calif.) according to the manufacturer's instructions. The integrity of RNA was verified by denaturing gel electrophoresis. Total RNA was linearly amplified using a modification of the Eberwine method (see Van Gelder et al., Proc. Natl. Acad. Sci. U.S.A. 87: 1663-1667, 1990). Table 2 catalogs the clinicopathologic features of the tumor samples studied.

TABLE 2
Clinicopathologic features of tumor samples

| Variable | BRCA1-linked | | BRCA2-linked | | Sporadic | |
|---|---|---|---|---|---|---|
| Number of samples | 18 | | 16 | | 27 | |
| Median Age* (SD) | 50 | (11) | 60 | (9) | 69 | (11) |
| Stage | | | | | | |
| I | 2 | (11.1%) | 0 | | 0 | |
| II | 0 | | 2 | (12.5%) | 3 | (11.1%) |
| III | 11 | (61.1%) | 12 | (75%) | 24 | (88.9%) |
| IV | 5 | (27.8%) | 2 | (12.5%) | 0 | |
| Grade | | | | | | |
| 1 | 0 | | 0 | | 0 | |
| 2 | 4 | (22.2%) | 6 | (37.5%) | 8 | (29.6%) |
| 3 | 14 | (77.8%) | 7 | (43.8%) | 16 | (59.3%) |
| No. | 0 | | 3 | (18.7%) | 3 | (11.1%) |
| Histology** | | | | | | |
| Serous | 9 | (50%) | 12 | (75%) | 16 | (59.3%) |
| Endometrioid | 3 | (16.7%) | 0 | | 2 | (7.4%) |
| Mucinous | 0 | | 0 | | 0 | |
| Clear Cell | 2 | (11.1%) | 0 | | 0 | |
| Adenocarcinoma NOS | 3 | (16.7%) | 3 | (18.8%) | 9 | (33.3%) |
| other | 1 | (5.5%) | 1 | (6.2%) | 0 | |

*F test, P = .0002/Data are the median +/− standard deviation.
**Chi test P value for differences in histology among tumor groups = 0.17
NOS = not otherwise specified

cDNA Microarrays: The cDNA microarrays consisted of 7,651 total features representing different (non-redundant) genes, and were manufactured at the National Cancer Institute microarray facility.

Total RNA was reverse-transcribed by using a 63 nucleotide synthetic primer containing the T7 RNA polymerase binding site (5′-GGCCAGTGAATFGTAATACGACTCACTATAGGGAGGCGG(T)₂₄-3′ (SEQ ID NO: 1). Second strand cDNA synthesis (producing double-stranded cDNA) was performed with RNase H, Escherichia coli DNA polymerase 1, and E. coli DNA ligase (Invitrogen, Carlsbad, Calif.). After cDNA was blunt-ended with T4 DNA polymerase (Invitrogen, Carlsbad, Calif.), it was purified by extraction with a mixture of phenol, chloroform, and isoamyl alcohol and by precipitation in the presence of ammonium acetate and ethanol. The double-stranded cDNA was then transcribed using T7 polymerase (T7 Megascript Kit, Ambion, Austin, Tex.), yielding amplified antisense RNA that was purified using RNeasy mini-columns (Qiagen, Valencia, Calif.). Pooled total RNA from two SV40 immortalized ovarian surface epithelial cell-lines (IOSE) was amplified and used as reference for cDNA microarray analysis.

Four μg of amplified RNA was reverse transcribed and directly labeled using cyanine 5-conjugated dUTP (tumor RNA) or cyanine 3-conjugated dUTP (IOSE RNA, provided by Dr. Jeff Boyd, Memorial Sloan-Kettering). Hybridization was performed in a solution of 5×SSC and 25% formamide for 14-16 hours at 42° C. Slides were washed, dried, and scanned using an Axon 4000a laser scanner (Axon Instruments, Inc., Union City, Calif.).

Imaging and I.M.A.G.E. Analysis: Fluorescence intensities at the immobilized targets were measured by using an Axon GenePix Scanner and Genepix Pro 3.0 analysis software (Axon Instruments, Union City, Calif.). The raw data were then uploaded to a relational database maintained by the Center for Information Technology at the National Institutes of Heath. The cDNA clones are identified by their Integrated Molecular Analysis of Genomes and their Expression Consortium (I.M.A.G.E.) clone number.

Amplification of RNA: The first strand of RNA was synthesized, by adding 1-3 μg of total RNA into a reaction tube (e.g., Eppendorf, or other container of suitable size), adding 1 μl T7-(dT)₂₄ primer (2 μg/μl), and bringing to a volume of 20 μl with nuclease-free water. The reaction was incubated at 70° C. for 10 minutes, then spun briefly in a centrifuge and placed on ice. Four μl 5× first strand cDNA buffer was added, then 2 μl 0.1M DTT, 2 μl 10 mM dNTP mix (Amersham-Pharmacia, Piscataway, N.J. 08855-1327 USA), 1 μl RNasin (Promega, Madison, Wis. 53711 USA), and 2 μl Superscript II. The reaction was mixed well and incubated at 42° C. for 1 hour. The tube was centrifuged briefly, and placed on ice. To synthesize the second strand, 91 μl DEPC-treated water was added, then 30 μl second strand buffer, 3 μl10 mM dNTP mix, 4 μl DNA Polymerase I (10U/μl), 1 μl DNA Ligase (10U/μl), and 1 μl RNAse H (2U/jμl). The final reaction volume equaled 150 μl. Next, the tube was gently tapped to mix, then briefly centrifuged. The tube was incubated at 16° C. for two hours, then 2 μl (10U) T4 DNA Polymerase was added. The tube was cooled for five minutes at 16° C., then the reaction stopped with 10 μl of 0.5 M EDTA. Ten μl of 1M NaOH were added, then the reaction was incubated at 65° C. for 10 minutes. The solution was neutralized by addition of 25 μl Tris-HCl (pH=7.5).

Clean Up of Double Stranded cDNA: The Phase Lock Gel (PLG) was pelleted in a microcentrifuge at maximum speed for 30 seconds. 198 μl (equal volume) of (25:24:1) Phenol:chloroform:isoamyl alcohol (saturated with 10 mM Tris-HCl pH 8.0/1 mM EDTA) was added to the final DNA synthesis preparation (198 μl) to a final volume of 396 μl. The solution was mixed well by pipetting up & down vigorously. The entire cDNA-phenol/chloroform mixture was transferred to the PLG tube, and microcentrifuged at maximum speed for two minutes. The aqueous supernatant was transferred to a new 1.5 ml tube, and 1 μl linear acrylamide was added. 0.5 volumes of 7.5M Ammonium Acetate+2.5 volumes (include the added Ammonium Acetate) of 95% ethanol stored at −20 to the sample was added and the solution was vortexed, then centrifuged at maximum speed in a microcentrifuge at room temperature for 20 minutes. The supernatant was removed and the pellet was washed with 0.5 ml of 80% ethanol. The solution was centrifuiged at maximum speed for 5 minutes at room temperature. The 80% ethanol was poured off, and the 80% ethanol wash repeated. The pellet was air dried for approximately 15 minutes, then resuspended in 16 pi of nuclease-free water.

In Vitro Transcription: Using an Ambion T7 Megascript kit (Ambion, Austin, Tex. 78744-1832, USA), the manufacturer's instructions were followed to create a 40 μl reaction (i.e., the 20 μl standard reaction was doubled and incubated at 37° C. for 4-5 hours). The reaction was assembled at room temperature using 16 μl of template double-stranded DNA, to avoid the precipitation of spermidine, which can occur if done on ice. Four μl of 10× reaction buffer were added, then 4 μl of ATP solution (75 mM T7), 4 μl of CTP solution (75 mM T7), 4 μl of GTP solution (75 mM T7), 4 μl of UTP solution (75 mM T7), 4 μl of Enzyme Mix, and the reaction was incubated at 37° C. for 5-6 hours. Sixty μl nuclease-free water was added to bring the total volume up to 100 μl. The RNA was “cleaned” using the “RNA clean-up” protocol provided in the Qiagen RNeasy Mini Handbook, May 1999, pp. 48-49, Qiagen, Valencia Calif. RNA was eluted with 30 μl of nuclease-free water, and the optical density ratio was measured (the sample should have an optical density of greater than 1.8 when measured at 260/280 nanometers). The expected yield from this preparation was ten times the starting amount of total RNA, and the RNA was then ready for use in generating probe for microarrays using total RNA (see below).

Second Round Amplifications: 0.5-1.0 μg of amplified RNA were resuspended in 11 μl ultrapure water.

First Strand Synthesis: One μl Random hexamer (1 mg/ml) was added and the reaction was incubated at 70° C. for 10 minutes, then chilled on ice, then allowed to equilibrate at room temperature for 10 minutes. Four μl 5× First strand cDNA buffer, 2 μl 0.1M DTT, 2 μl 10 mM dNTP mix, 1 μl RNasin were added, and the reaction was mixed incubated at 42° C. for 2 minutes. Two μl Superscript II were added, and the reaction was mixed well and incubated at 42° C. for 1 hour. One μl RNAse H was added, and the reaction was incubated at 37° C. for 20 minutes, then heated to 95° C. for 2 minutes to quell the reaction, then chilled on ice.

Second Strand Synthesis: One μl T7-oligodT primer (0.5 mg/ml) was added, and the reaction was incubated at 70° C. for 5 minutes and at 42° C. for 10 minutes. Then, 91 μl DEPC treated H₂O were added, then 30 μl Second strand buffer, 3 μl10 mM dNTP mix, 4 μl DNA Polymerase I (10U/μl), 1 μl DNA Ligase (10U/μl), and 1 μl RNAse H (2U/μl) to a final volume of 150 μl. The tube was tapped gently to mix, then briefly centrifuged. The reaction was incubated at 16° C. for two hours, then 2 μl (10U) T4 DNA Polymerase were added, and the reaction was cooled for 5 minutes at 16° C. The reaction was stopped with 10 μl of 0.5 M EDTA, then 10 μl of 1M NaOH were added. The reaction was incubated at 65° C. for 10 minutes, then neutralized by addition of a solution with 25 μl Tris-HCl (pH=7.5). The protocol for “Clean Up of Double Stranded cDNA” and “In Vitro Transcription” was followed to generate cDNA for use in preparation of the probe for microarray hybridization.

Preparation of Probe and Microarray Hybridization Using Amplified RNA: To prepare the probe, reverse transcription labeling reaction mixes were created for each probe containing the component Random Primer (InVitrogen, Carlsbad, Calif. 92008, USA). Three μg/μl in 2 μl were added, then 5-6 μg amplified RNA. The reaction was brought to a final volume of 17 μl with water, then incubated at room temp for 10 minutes. To each probed, 5× first strand buffer in 8 μl, 20× lowT-dNTP mix in 2 μl, 0.1 M DTT in 4 μl, RNAsin in 1 μl, Cy-3 or Cy-5 dUTP (NEN Life Science, Boston, Mass. 02118-2512 USA) in 4 μl, and SuperScriptI] (GIBCO-BRL, InVitrogen Corporation, Carlsbad, Calif. 92008 USA) enzyme in 2 μl was added. The reaction was incubated at 42° C. for 60 minutes, then 5 μl of 500 mM EDTA and 10 μl of 1M NaOH were added. The reaction was incubated at 65° C. for 15 minutes to hydrolyze residual RNA, then cooled to room temperature. Twenty-five 25 μl of 1M Tris-HCl (pH7.5) was added to neutralize pH.

Probe Cleanup: 500 μl of 1×TE were added to a Microcon-YM30 column and the column was spun at 13,000 rpm for 5-6 minutes to wash the column. Membrane integrity was checked by looking into the top insert, to confirm that a thin film of TE (˜50 μl) covered the membrane. 400 μl 1×TE was added to each of the sample tubes and all contents were transferred to the washed Microcon-YM30 column (Amicon, Millipore Corp., Bedford, Mass.). The column was spun at 13,000 rpm for 5-6 minutes until approximately 50 μl was left on the membrane. The column was checked for dye crystals along the edge of the column membrane, which indicated that the probe was likely to be good. 450 μl 1×TE were added to the column and the column was spun down to ˜50 μl as above. The presence of crystals was confirmed. The Cy-3 labeled probe was placed into a clean tube, and the column was spun at 14,000 rpm for 1 minute to elute the probe. The Cy-3 labeled probe was added to the Cy-5 labeled probe in the column, and approximately 450 μl 1×TE was added to the column. The column was spun at 13,000 rpm until approximately 13-14 μl of combined probe remained on the membrane, which was checked with a pipette. The combined probe was inverted into a clean tube, and spun at 14,000 for 1 minute to elute. The probe (14 μl) was transferred into a clean Eppendorf tube and stored at 4° C. until used in the hybridization reaction.

Probe Hybridization: Twenty μl of water were added to each humidifying well in the Hybridization Chamber (to maintain humidity). Then, 40 μl of prehybridization buffer (5×SSC, 0.1% SDS, 1% BSA (Sigma) warmed to 42° C.) were placed in the center of the slide and the cover slip was placed on the slide, taking care to prevent bubble accumulation beneath the slip. The margin clamps on the Hybridization Chamber were firmly attached, and the chamber was incubated at 42° C. for least 1 hour. The slide was washed in distilled water for 2 minutes, followed by isopropanol for 2 minutes. The slide was dried in a centrifuge (5804R, Eppendorf) at 705 rpm (˜70×g) for 4 minutes, then prepared for hybridization as discussed above. The slide was hybridized within 1 hour of the prehybridization step. Two 2 μl COT1-DNA (Hoffman La Roche, Nutley, N.J. 07110 USA) (1 μg/μl), 2 μl polyA (Sigma) (8-10 μg/μl), and 2 μl yeast tRNA (Sigma, Ronkonkoma, N.Y. 11779 USA) (4 μg/μl) were mixed with the probe. Then, the probe was denatured for 1 minute at 100° C., placed briefly on ice to cool the reaction, and spun down in a centrifuge. Twenty μl of 2× hybridization buffer (50% formamide, 10×SSC, 0.2% SDS, warmed to 42° C.) were added to the denatured probe, mixed well (taking care to minimize bubble formation) and kept at 42° C. until ready to spot on the slide. The hybridization chamber was prepared as in the prehybridization step with 20 μl of distilled water in each well. The slides were placed face-up in the chambers, and the probe was hybridized with the slide for 14-16 hrs at 42° C.

Slide Washes: The margin clamps on the Hybridization Chamber were carefully removed to prevent water from seeping in and contaminating the array. The slide was removed from the chamber, held with forceps and the cover slip allowed to fall off into the solution containing 2×SSC, 0.1% SDS. The slide was washed for 4 minutes in 1×SSC, 0.1% SDS, for 4 minutes in 0.2×SSC, and for 1 minute in 0.05×SSC. The slide was spun dry in a centrifuge at 705 rpm (approximately 70×g) for 4 minutes. If water droplets were seen on the slide, it was spin again for another 4 minutes. Exposure to light was minimized by placing the dried slides in a slide box until ready for scanning.

Statistical Analysis: The logarithmic expression ratios for the spots on each array were normalized by subtracting the median logarithmic ratio for the same array. Data were filtered to exclude spots with size less than 25 μm, intensity less than 2 times background or less than 300 units in both red and green channels, and any poor-quality or missing spots. In addition, any features found to be missing or flagged in greater than 10% of the arrays were not included in the analysis. Application of these filters resulted in the inclusion of 6,445 of the total 7,651 features in subsequent analyses. Statistical comparison between tumors groups was performed using the “BRB Array Tools” software (developed by Dr. Richard Simon and Amy Peng, Biometrics Research Branch, Division of Cancer Treatment and Diagnosis, NCI, USA). A modified F-test is run on each gene's log-ratio values, and the significance of that gene is determined with P<0.0001 considered significant. This stringent P value is selected in lieu of the Bonferroni correction for multiple comparisons, which was deemed excessively restrictive (FIG. 1) (see Bland and Altman, B.M.J; 310: 170, 1995).

Semi-quantitative PCR: Five samples from each tumor group (BRCA-1, BRCA-2, sporadic) were selected at random. For each sample, 3.5 μg of total RNA was reverse-transcribed using oligo dT primers and 400 units of Superscript II reverse transcriptase (Invitrogen, Carlsbad, Calif.) in the presence of all four deoxyribonucleoside 5′-triphosphates (each at 10 mM) (InVitrogen, Carlsbad, Calif.) and 40 units of RNAse inhibitor (Promega, Madison, Wis.). Reverse transcription was performed in a total reaction volume of 40 μl, of which 1 μl was subsequently used for each PCR reaction. Preliminary experiments were performed to identify optimal cycle number for each gene. Thirty cycles was found to be optimal amplification for all amplified RNAs except for HLA-DRB1 and CD74, which were amplified for 26 cycles. Polymerase chain reaction was performed using the GeneAmp PCR kit (PE Applied Biosystems, Foster City, Calif.) according to the manufacturer's instructions. Representative gene specific primer sequences are shown in Table 3:

TABLE 3
Primer pairs for RNA amplification.

| | |
|---|---|
| Gene specific primer pair | SEQ ID NO. |

| | | |
|---|---|---|
| ACTB | 5′-ATGTGGATCAGCAAGCAGGA-3′ | SEQ ID NO: 2 |
| | 5′-GGTGGCTTTTAGGATGGCAA-3′ | SEQ ID NO: 3 |
| HE4 | 5′-TTCGGCTTCACCCTAGTCTCA-3 | SEQ ID NO: 4 |
| | 5′-AGAGGGAATACAGAGTCCCGAA-3′ | SEQ ID NO: 5 |
| ZFP36 | 5′-ACCCTGATGAATATGCCAGCA-3′ | SEQ ID NO: 6 |
| | 5′-GCTACTTGCTTTTGGAGGGTA-3′ | SEQ ID NO: 7 |
| RGS1 | 5′-GACTCTTATCCCAGGTTCCTCA-3′ | SEQ ID NO: 8 |
| | 5′-TGACTCCCTGGTTTTAAGAGCA-3′ | SEQ ID NO: 9 |
| CD74 | 5′-CCAGTCCCCATGTGAGAGCA-3′ | SEQ ID NO: 10 |
| | 5′-AGCTGATAACAAGCTTGGCTGA-3′ | SEQ ID NO: 11 |
| TOP2A | 5′-TGTCCCTCCACGAGAAACAGA-3′ | SEQ ID NO: 12 |
| | 5′-CGTACAGATTTTGCCCGAGGA-3′ | SEQ ID NO: 13 |
| HLA-DRB1 | 5′-GCGAGTTGAGCGTAAGGTGA-3′ | SEQ ID NO: 14 |
| | 5′-TTGAAGATGAGGCGCTGTCA-3′ | SEQ ID NO: 15 |

Amplified RT-PCR products were visualized on an agarose gel stained with ethidium bromide. The intensity of each band was an indicator of the quantity of DNA, as previously amplified by PCR. Thus, the intensity served as an indirect measure of the starting amount of the RNA amplified from the respective gene in each sample. Intensity was quantified using an ultraviolet light source and Alpha Imager software (Alpha Innotech Corp, San Leandro, Calif.). In addition to the above-mentioned tumor samples, sqRT-PCR evaluation of selected genes was also performed on the IOSE RNA for comparison.

### Results

Global assessment of Gene expression differences among tumor groups: Prior to investigating specific inter-group differences, the overall patterns of gene expression in the three tumor types (BRCA-1, BRCA-2, sporadic) were assessed. Multidimensional scaling (MDS), based on the expression levels of all 6,445 filtered genetic elements in the microarray, revealed that BRCA1- and BRCA2-linked tumors have distinct molecular profiles. In contrast, the sporadic samples showed a more heterogeneous distribution pattern, with many patterns clustering near the patterns of BRCA1-linked or BRCA2-linked samples (FIG. 1A). The MDS results suggested that the BRCA1- and BRCA2-associated groups would be the most different and that gene expression patterns for each of the BRCA groups and the sporadic tumors would have fewer differences. In support of this hypothesis, only a few genes showed statistically significant (P<0.0001) differential expression between the sporadic tumors and the BRCA1- or BRCA2-linked tumors, whereas 110 genes were differentially expressed between BRCA1-linked and BRCA2-linked tumors (FIG. 1B). In addition 34 EST sequences were differentially expressed between BRCA1- and BRCA2-linked tumors. The group of 144 total markers that were differentially expressed between BRCA1- and BRCA2-type tumor cells compared to normal ovarian epithelial cells is listed in Table 9 (see Addendum).

Differential gene expression among all three groups was also performed, which identified 60 genes and 3 EST sequences whose expression segregated BRCA1-linked, BRCA2-linked, and sporadic tumors (modified F test, wherein P<0.0001). Fifty-one of these 63 genes and EST sequences were also among the statistically significant discriminators of BRCA1 and BRCA2 tumors, highlighting the distinct gene expression profiles of these two groups. In addition, the expression profile of the combined BRCA1- and BRCA2-linked group was remarkably similar to that of the sporadic tumors, as demonstrated by only three genes showing differential expression (P<0.0001) between these groups [PSTP1P1 (SEQ ID NO: 538-540), IDH2 (SEQ ID NO: 541-542), and PCTK1 (SEQ ID NO: 527-528)]. These observations were in agreement with the multidimensional scaling analysis and demonstrated that, in terms of the overall pattern of gene expression, the BRCA1- and BRCA2-linked tumors are distinct from one another. Furthermore, the gene expression profiles of the sporadic tumors appear to share features of either BRCA1- or BRCA2-linked cancers, and these sporadic tumors are referred to herein as BRCA1-type or BRCA2-type sporadic ovarian tumors.

The group of 144 nucleic acid molecules listed in Table 9 was further investigated using hierarchical clustering (FIG. 2A, B). As expected, the BRCA-associated tumors showed distinct and contrasting expression profiles (FIG. 2A). Strikingly, the sporadic samples also segregated into two groups based on the expression patterns of the same 144 genes, exhibiting sporadic sample had a molecular profile similar to that of either the BRCA1- or the BRCA2-linked tumors. This observation was illustrated by hierarchical clustering of all samples, revealing distinct “BRCA1-type” and “BRCA2-type” clusters (FIG. 2A). This clustering further demonstrates that sporadic tumors (which do not contain the BRCA1 or BRCA2 mutations) can often be classified as BRCA1-type or BRCA2-type. Classification of sporadic tumors into these subtypes may provide guidance in treating the patient. For example, a subject who has a BRCA1-type or BRCA2-type sporadic tumor may be treated similarly to a subject who has a BRCA1-linked or BRCA2-linked tumor. The identification of BRCA1- or BRCA2-type sporadic tumors also allows tumors (or subjects) to be selected for specific drug regimens that are particularly effective with the associated mutation type.

Color-coding is usually used to represent the relative transcript expression ratio, as measured by cDNA microarray analysis. Red customarily indicates the maximum point in gene expression, green the minimum, and levels closer to the mean approach black

To ensure that the BRCA-linked samples were not biasing the observed clustering patterns, the hierarchical architecture of gene expression in sporadic tumors was examined separately. Even in the absence of the BRCA-linked samples, two distinct cluster phenotypes were observed, each comprised of those sporadic samples that previously grouped with BRCA1- and BRCA2-linked tumors (FIG. 2B). Tumor histology and patient age were also evaluated for possible confounding effects on the observed BRCA1-type and BRCA2-type clusters. Neither variable was found to influence clustering patterns (FIG. 2A, 2B).

Genes differentially expressed between BRCA1- and BRCA2-linked ovarian carcinomas: The analysis of overall gene expression patterns established that the same genes whose expression differentiated BRCA1 and BRCA2-linked tumors, also identified two major sub-populations of sporadic cancers (FIG. 3). As such, these nucleic acids are believed to represent important mediators of common genetic pathways in ovarian cancer and/or carcinogenesis. Many of these genes are involved in important cellular functions including signal transduction, RNA processing and translation, chemokine signaling and immune modification, and DNA repair. By way of example, the BRCA1-associated tumors were characterized by higher AKT1 (SEQ ID NO: 504-506) and lower PTEN (SEQ ID NO: 507-509) relative expression. In addition UBL1 (SEQ ID NO: 510-512) (also known as SUMO-1 and sentrin) was more highly expressed in BRCA1-associated tumors. This molecule interacts with RAD51 and RAD52 and has been proposed to have a regulatory role in homologous recombination (see Li et al., Nuc. Ac. Res: 28: 1145-1153, 2000). The preferential expression of UBL1 (SEQ ID NO: 510-512) in the BRCA1-linked samples may prove to be relevant to possible differences in DNA repair actions of the BRCA tumor suppressor genes.

By way of example, the BRCA2-linked tumors showed higher relative expression of WNT2 (SEQ ID NO: 513-514 and SFRP4 (SEQ ID NO: 515-517), which are members of the wnt-β-catenin-TCF signaling pathway. Another notable observation is that both BRCA1- and BRCA2-linked tumors showed preferential expression of proto-oncogenes commonly altered in hematologic malignancies. BRCA1 tumors showed higher expression levels of RUNX1(SEQ ID NO: 518-520)/AML1, while BRCA2-associated samples showed preferential expression of TAL1 (SEQ ID NO: 521-523)/SCL. Both of these oncogenes are transcription factors involved in proliferation, and their preferential expression in BRCA1- and BRCA2-linked tumors may indicate that the activation of such a “proliferation driver” is a necessary step in ovarian carcinogenesis.

Gene expression differences between BRCA-linked and sporadic tumors: Nine non-redundant genes showed significant differential expression between BRCA1-linked and sporadic tumors [CD72 (SEQ ID NO: 805), SLC25A11 (SEQ ID NO: 544), LCN2 (SEQ ID NO: 545-547), PSTP1P1 (SEQ ID NO: 538-540), SIAHBP1 (SEQ ID NO: 543), UBE1 (SEQ ID NO: 533), WAS (SEQ ID NO: 524-526), IDH2 (SEQ ID NO: 541-542), PCTK1 (SEQ ID NO: 527-528), P<0.0001, FIG. 4A. A noteworthy observation was that three of these genes, WAS (SEQ ID NO: 524-526), PCTK1 (SEQ ID NO: 527-528), and UBE1 (SEQ ID NO: 533), have all been mapped to the Xp11.23 and all were higher expressed in the BRCA1-linked tumors. This observation seemed unlikely to be explained by chance alone as only 35 of the total 6,445 filtered spots (0.5%) on the microarray represent genes mapped to Xp11. To further investigate this pattern, a larger group of 53 genes was considered for differential expression between BRCA1-linked and sporadic tumors under the less stringent significance level of P<0.001. Among this group three additional genes, SMC1L1 (SEQ ID NO: 530), ARAF1 (SEQ ID NO: 531-532), and EBP (SEQ ID NO: 529), were discovered that also mapped to the Xp11.23 locus and also showed higher mean expression in BRCA1-associated samples (FIG. 4D). Thus, six of fifty-three genes differentially expressed between BRCA1-linked and sporadic samples (P<0.001) mapped to Xp11.23 and all showed higher mean expression in BRCA1-linked tumors. In silico analysis of the location of these genes revealed that they are all confined to a 5-Mb region of DNA in Xp11.23 (Ensemble database, Prous Science, Philadelphia, Pa. 19102, U.S.A.).

The comparison between BRCA2-linked and sporadic tumors revealed only two genes with differential expression among these groups at the significance level of P<0.0001 (FIG. 3). The gene designated as LOC51760 (SEQ ID NO: 534-535) is also known as B/K (encoding the brain/kidney protein) and is moderately homologous to the synaptotagmin family of vesicular transport molecules. The second differentially expressed gene encodes low-density lipoprotein-related protein-associated protein 1 (LRPAP1), also known as alpha-2-macroglobulin receptor-associated protein 1.

A further comparison consisted of investigating gene expression differences between the combined BRCA-linked group and the sporadic group, which revealed only three non-redundant, differentially expressed genes [PSTP1P1 (SEQ ID NO: 538-540), IDH2 (SEQ ID NO: 541-542), and PCTK1 (SEQ ID NO: 527-528), FIG. 4C. All three genes were among the group of genes that differentiated BRCA1-linked and sporadic samples. This finding is consistent with the observation that the RNA profiles of sporadic ovarian cancers share significant similarities with those of BRCA1-linked or BRCA2-linked tumors. It is believed that the similarities shown in the RNA profiles is a general characteristic that applies to gene and protein component profiles as well. The small number of differentially expressed genes obtained from the comparison of the combined BRCA group to the sporadic tumors is the result of the latter also consisting of BRCA1-type and BRCA2-type molecular classes.

Gene expression features distinguishing ovarian cancers from ovarian surface epithelial cells: Gene expression patterns common among all tumor types were investigated to identify genes that may be associated with the transformed state, i.e., genes commonly expressed in ovarian tumors irrespective of their hereditary or sporadic nature. Gene expression in all sixty-one primary tumor samples was compared to immortalized ovarian surface epithelial (IOSE) cells used as the common reference. Using the selection criterion of two-fold or greater expression ratio relative to the IOSE reference in at least two-thirds of all tumors, a list of 201 non-redundant genes and ESTs was generated. The top twenty-five overexpressed (IL8 (SEQ ID NO: 449-451), GRO1 (SEQ ID NO: 452-453), ALDH1A3 (SEQ ID NO: 454-456), MMP1 (SEQ ID NO: 457-459), OSF-2 (SEQ ID NO: 460-461), CDC2SB (SEQ ID NO: 462-464), FLNA (SEQ ID NO: 465-467), TFP12 (SEQ ID NO: 468-469), FGF2 (SEQ ID NO: 470-472), CD44 (SEQ ID NO: 473-475), DYT1 (SEQ ID NO: 476-477), UCHL1 (SEQ ID NO: 478), FGF2 (SEQ ID NO: 470-472), PLAU (SEQ ID NO: 479-480), LDHA (SEQ ID NO: 256), PTGS2 (SEQ ID NO: 481-483), PRNP (SEQ ID NO: 484-486), MT1X (SEQ ID NO: 487-488), UGB (SEQ ID NO: 489-490), PBEF (SEQ ID NO: 491-493), TXNRD1 (SEQ ID NO: 494-496), NTS (SEQ ID NO: 497-499), PTGS2 (SEQ ID NO: 481-483), MT2A (SEQ ID NO: 500-502), ZNF220 (SEQ ID NO: 503)) and twenty-five down-regulated (FLJ22174 (SEQ ID NO: 30-31), DDR1 (SEQ ID NO: 74-76), SERPINF2 (SEQ ID NO: 18-19), HLA-DRB1 (SEQ ID NO: 87-88), IFITM2 (SEQ ID NOS: 55-57, 58-59), HGF (SEQ ID NO: 174-175), SORL1 (SEQ ID NO: 149-151), CP (SEQ ID NO: 83-84), HLA-DRA (SEQ ID NO: 94-96), BRF2 (SEQ ID NO: 190-192), ABCB1 (SEQ ID NO: 164-166), GIP3 (SEQ ID NO: 68-69), RGS1 (SEQ ID NO: 122-123), IFITM1 (SEQ ID NOS: 50-51, 52-54), FOS (SEQ ID NO: 133-135), PPP1R7 (179-180), HLA-DPA (SEQ ID NO: 97-99), HLA-DRB5 (SEQ ID NO: 85-86), TLR3 (SEQ ID NO: 199-201), ZFP36 (SEQ ID NOS: 167-168, 169-171, 172-173), SGK (SEQ ID NO: 176-178), HLA-DRB1 (SEQ ID NO: 87-88), HE4 (SEQ ID NO: 60), CD74 (SEQ ID NO: 89-91, CD24 (SEQ ID NO: 181-182)) named genes (by order of magnitude) are presented in FIGS. 4A and 4B. This analysis revealed two potentially significant functional groups of genes to be overexpressed in ovarian cancers. The first group consisted of several of the genes that have all been previously shown to be interferon-inducible (HLA-DRB1 (SEQ ID NO: 87-88), HLA-DRB5 (SEQ ID NO: 85-86), HLA-DRA (SEQ ID NO. 373-374), HLA-DPA (SEQ ID NO: 97-99), CD74 (SEQ ID NO: 89-91), IFITM1 (SEQ ID NOS. 50-51, 52-54), and IFITM2 (SEQ ID NOS: 55-57, 58-59), as indicated by italics in FIGS. 4A and 4B). The second group consisted of immediate-early response genes (BRF2, ZFP36, SGK, and FOS). In addition, several genes previously reported to be overexpressed in ovarian epithelial tumors were present in the list of genes overexpressed in tumors relative to the IOSE cells (FIGS. 4A and 4B). Elevated levels of CLU, CD24, and MUC1 were also observed. These results identify additional potential markers of ovarian cancer. Table 9 lists the 144 nucleic acids that showed significantly elevated expression in ovarian cancer. These genes were selected based on consistency across all the pooled experiments and a significant difference in the average expression in the 40 independent samples, using a criteria of a tumor-to-ovarian surface epithelial cell line ratio of two or greater in at least 66% of all tumors.

### EXAMPLE 2

### Semiquantitative RT-PCR Confirms and Complements cDNA Microarray Data

This example describes how the results found in the previous example were confirmed using semiquantitative RT-PCR.

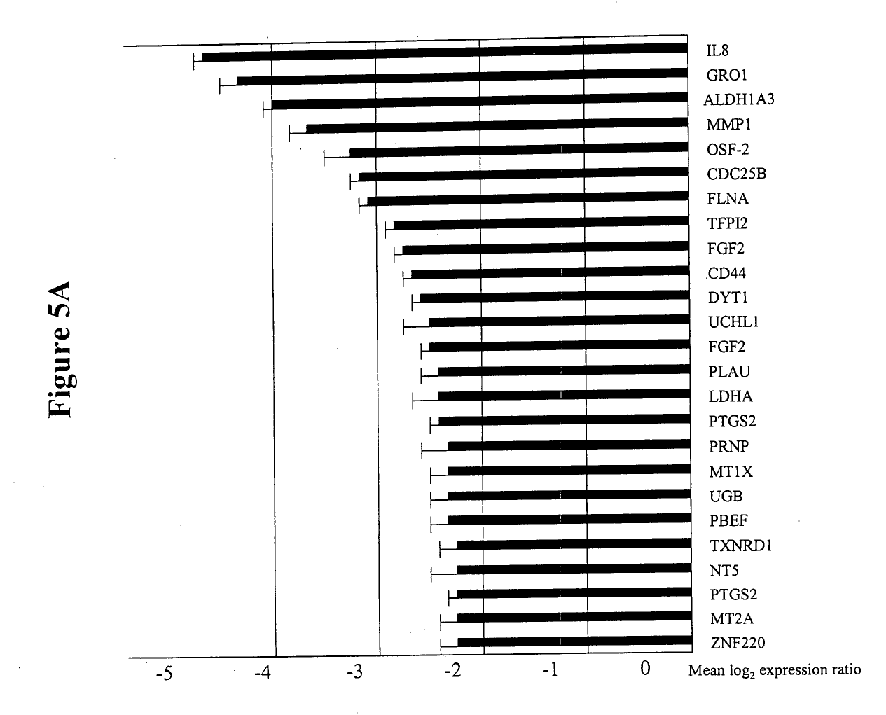

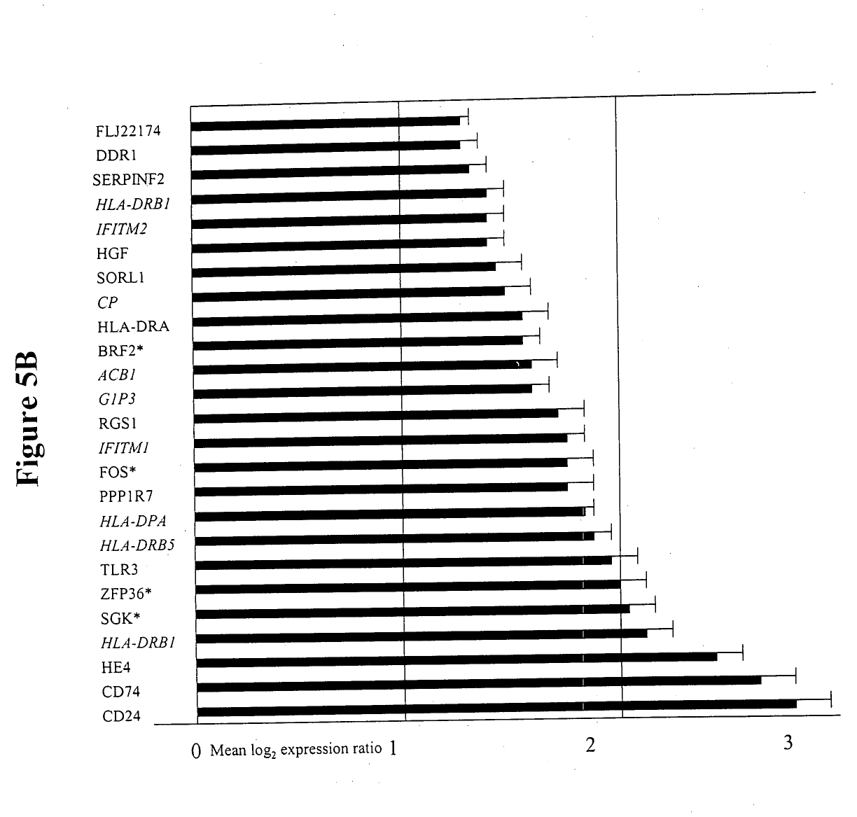

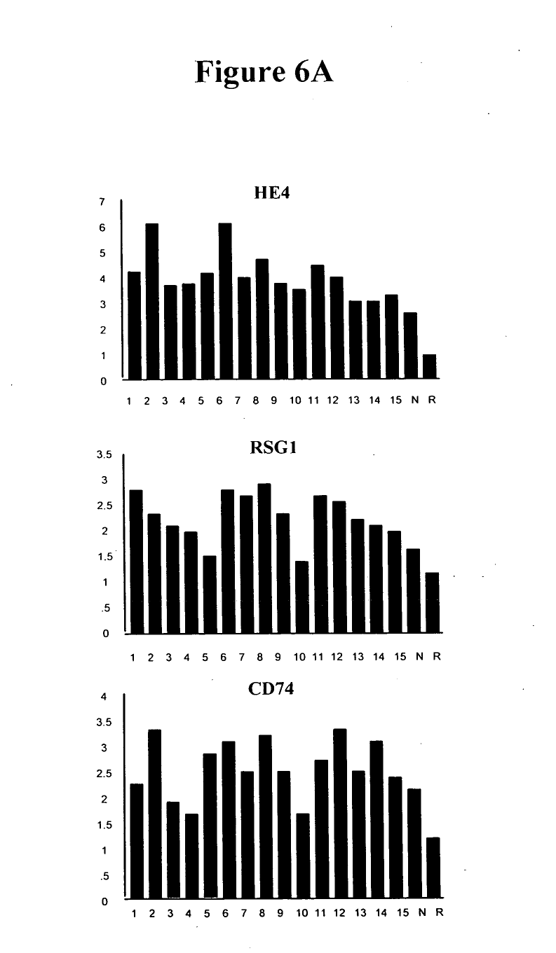

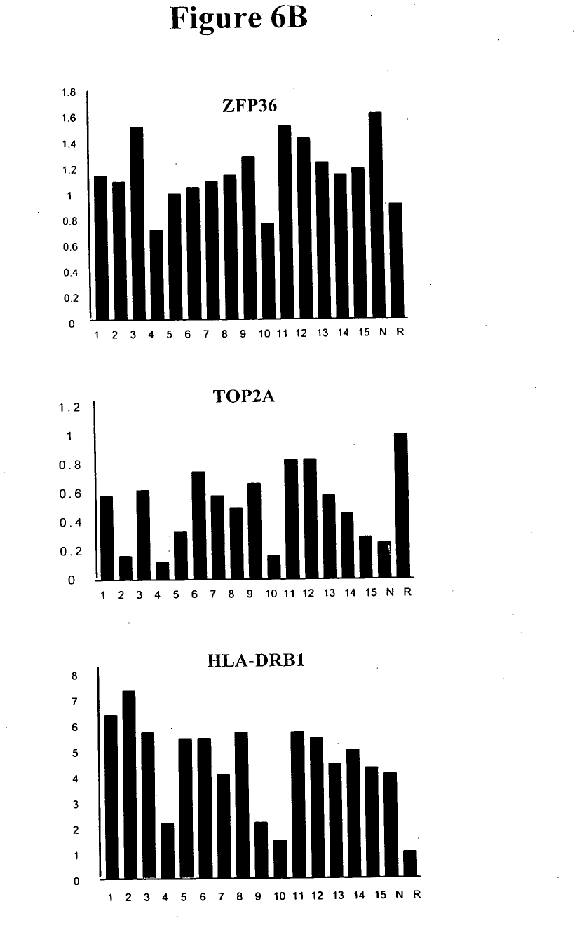

To validate the array data, semiquantitative RT-PCR (sqRT-PCR) analysis of several mRNAs was performed in a representative subset of tumors consisting of five BRCA1-linked, five BRCA2-linked, and five sporadic RNA samples. The tumor samples were randomly selected. The expression of TOP2A (SEQ ID NO: 448), RGS1 (SEQ ID NO: 398, CD74 (SEQ ID NOS: 89-91, 92-93), HE4 (SEQ ID NO: 60), HLA-DRB1 (SEQ ID NO: 87-88), and ZFP36 (SEQ ID NO: 167-168, 169-171, 172-173) were evaluated using sqRT-PCR, with β-actin as a normalizing control. Because data obtained from cDNA microarrays is in the form of relative expression ratios between tumors and the reference, RNA from IOSE cells and a histologically normal, postmenopausal ovarian RNA sample in the sqRT-PCR experiments was included for comparison. The results of these sqRT-PCR experiments were consistent with the cDNA microarray relative expression data for all six genes evaluated (FIGS. 5A and 5B). As anticipated from the microarray results (FIG. 4), HE4 expression was consistently elevated in all fifteen tumor samples compared to IOSE reference cell-line and normal ovary (FIGS. 5A and 5B). Invariant chain genes, also known as CD74 and RGS1, were overexpressed in the majority of tumors as indicated by microarray analysis (FIG. 4). Both were also found to have increased expression in the majority of tumors as evaluated by sqRT-PCR (FIGS. 5A and 5B). The expression of TOP2A was found to be highest in the reference IOSE RNA. Furthermore, compared with the expression level in the normal postmenopausal ovary, twelve of the fifteen tested tumor samples showed elevated and variable TOP2A gene expression.

Several members of the immediate-early response cascade showed elevated expression in tumors as compared to IOSE cells in the microarray experiments (as indicated by the notation * in FIGS. 4A and 4B); however, some of these genes have previously been shown to have lower expression in ovarian cancer compared to normal ovary (see Welsh et al. Proc. Natl. Acad. Sci. U.S.A. 98: 1176-1181, 2001, and Wang et al., Gene 229: 101-108, 1999).

This discrepancy suggested that the elevated relative ratio observed in these experiments may be driven by low expression levels of these genes in IOSE cells grown in culture. In order to test this hypothesis, sqRT-PCR was used to compare ZFP36 (an immediate-early gene also known as G0S24 and Tis11) expression in tumors to that of normal ovary and IOSE cells. As suspected, normal ovary had one of the highest expression levels of ZFP36, followed by that of the majority of tumors (FIG. 5A), while the lowest expression level was observed in the IOSE cells.

In addition to statistical analysis, multidimensional scaling (MDS) and hierarchical clustering techniques using a correlation metric and average linkage were used for evaluating overall gene expression (see Eisen et al., Proc. Natl. Acad. Sci. U.S.A. 95: 14863-14868, 1998).

### EXAMPLE 3

### Identification of Additional Genes with Altered Expression in Ovarian Cancer

This example provides a description of how additional disclosed ovarian cancer-related nucleic acid molecules were identified. These ovarian cancer-related molecules show differences in expression in subjects having ovarian cancer compared to expression in normal ovarian surface epithelial cells.

Using a different microarrays and methods essentially similar to those described above in Example 1, thirty-one ovarian epithelial cancers were compared to two normal postmenopausal ovarian samples. 141 additional ovarian cancer-related nucleic acid molecules were identified and further characterized (Tables 6 and 7, Addendum).

Methods and Materials:

Methods and materials were similar to those described in Example 1, except that different microarrays were used. The nucleic acids constituted 7,600 features, and representing different (non-redundant) transcripts including multiple known named genes and ESTs. The cDNA microarrays were constructed by Dr. Eric Chuang (Division of Radiation Oncology) at the Advanced Technology Center (Gaithersburg, Md. 20877). The genes represented on these arrays are composed of 7,600 cDNA clones and ESTs and are commercially available (Research Genetics, 2130 Memorial Parkway, Huntsvillle, Ala. 35801, U.S).

The nucleic acid molecule expression patterns of thirty-one ovarian epithelial cancers were compared to two normal postmenopausal ovarian samples. The tissues were analyzed once, as the correlation coefficient from previously repeated array experiments was shown to be 0.92-0.95. Each tumor and normal sample was directly compared to a “reference RNA” consisting of a mix of nine different human cell lines (Stratagene, La Jolla, Calif.), allowing for indirect comparison of gene expression in tumors and normal ovarian samples.

Hierarchcal clustering was performed as described above and as set forth in Eisen et al., Proc. Natl. Acad. Sci. U.S.A. 95: 14863-8, 1998.

Results

Systematically Altered Genes

Using these methods, two additional sets of nucleic acid molecules were identified that showed differential expression in subjects having ovarian cancer. Table 4 (see Addendum) provides a list of nucleic acid molecules that were found to be underexpressed in subjects having ovarian cancer, and their average gene log expression ratios. Table 5 (see Addendum) shows nucleic acid molecules that were found to be overexpressed in persons having ovarian cancer, and their average gene log expression ratios.

Genes underexpressed in ovarian tumors (see Table 4) may represent potential tumor suppressors. The induction of the expression of these genes through therapeutic means, for instance by induction through drug or gene therapy, may slow tumor growth and/or increase tumor cell death.

Among the 100 underexpressed genes were several oncogenes coding for proteins that are normally associated with the process of malignant transformation, 8ncluding JUN (SEQ ID NO: 137-138), KIT (SEQ ID NO: 298-299), and MAF (SEQ ID NO: 229-230). The lower expression of these genes in cancers compared to expression in non-cancerous subjects is unexpected and is believed to reflect novel effects unique to ovarian cancer. Additionally, CDKN1C (SEQ ID NO: 249), NBL1 (SEQ ID NO: 273), and ING1L (SEQ ID NO: 322) are recognized tumor suppressors, the downregulation of which may be involved in the process of tumor formation and/or progression. TGF beta cascade members TGFBR3 (SEQ ID NO: 216-218) and EBAF (SEQ ID NO: 294) (both shown herein to be underexpressed in ovarian cancer) present potential interest in light of the recent implication of the TGF beta pathway in normal and oral contraceptive-induced ovarian epithelial cell death and turnover (see Rodriguez et al., J. Natl. Can. Inst. 94(1): 50-60, 2002). Thus, downregulation of these nucleic acids may lead to inappropriate growth and possible transformation.

Genes that were overexpressed in ovarian tumors (Table 5) compared to normal tissue are believed to represent suitable targets for therapy and/or diagnosis, prognosis and staging of ovarian cancer. The decrease of the expression of these genes through therapeutic means, for instance by drug or gene therapy, presents a potential method of inhibition of ovarian cancer.

Among the fifty-nine overexpressed genes were several genes coding for proteins that are believed to be particularly promising as gene targets, including the following: SLP1 (Secretory leukocyte protease inhibitor) (SEQ ID NO: 340-341); SPP1 (Secreted phosphoprotein 1) (SEQ ID NO: 342); CKS1 (CDC28 protein kinase 1) (SEQ ID NO: 345-347); ZWINT(ZW10 interactor) (SEQ ID NO: 354); BF(B-factor, properdin) (SEQ ID NO: 343-344); MMP7 (Matrix metalloproteinase 7) (SEQ ID NO: 348-349); FOLR1 (Folate receptor 1) (SEQ ID NO: 364-365); KLK8 (Kallikrein 8) (SEQ ID NO: 368; CR1P1 (Cysteine-rich protein 1) (SEQ ID NO: 375; EYA2 (Eyes absent) (SEQ ID NO: 392-393); and PAX8 (Paired box gene 8) (SEQ ID NO: 350-351).

SLP1 is a particularly promising candidate as a potential ovarian cancer marker or detector. This protein has also been shown to be overexpressed in lung cancer (see Ameshima et al., Cancer 89(7): 1448-1456, 2000) and is detectable in the saliva, enabling non-invasive testing (see Shugars et al., Gerontology, 47(5): 246-253, 2001). MMP7 over-expression has been described in primary and metastatic gastric cancers (see Mori et al., Surgery, 131(1 Pt 2): S39-S47, 2002) as well as colorectal carcinomas (see Ougolkov et al., Gastroenterology. 122(1): 60-71, 2002). MMP7 appears to be involved in new blood vessel formation, which is a prerequisite for tumor growth (see Nishizuka et al., Cancer Lett. 173(2): 175-182, 2001). SPP1 (otherwise known as osteopondin) has also been associated with a number of malignancies (see Fedarko et al., Clin. Cancer Res. 12: 4060-4066, 2001) including a recent report showing higher expression in ovarian cancer (see Mok et al., J. Natl. Cancer Inst. 93(19) 1458-64, 2001). ZWINT is a newly discovered protein involved in kinetochore binding and centromere function (see Starr et al., J. Cell Sci. 13(Pt 11): 1939-1950, 2000). Properdin is involved in immune function and encodes complement factor B, a component of the alternative pathway of complement activation. CR1P1 is believed to be involved in zinc transport. Kallikrein 8 (also TADG14) is normally expressed in neural tissue, but appears to be altered such that it is highly expressed in ovarian cancers (see Underwood et al., Cancer Res. 59(17): 4435-4439, 1999). EYA2, named for its involvement in eye development, is an important developmental gene that is potentially important in ovarian cancer. EYA2 is located on the 20q13 chromosomal locus, which is the most frequently amplified chromosome region in ovarian cancers (see Tanner et al., Clin. Cancer Res. 5: 1833-1839, 2000). Other genes localized to the same 20q13 chromosomal region are BMP7, which is also involved in development, and SLP1 (discussed above), as well as HE4 (identified in Example 1, above), all of which show higher expression in ovarian tumors. Thus, the upregulation of these nucleic acids may in part be due to amplification of 20q13 in the tumors studied.

PAX8 is involved in thyroid differentiation and normal function (see Pasca et al., Proc. Natl. Acad. Sci. U.S.A. 97(24): 13144-13149, 2000). Furthermore, the folate receptor has been shown to be overexpressed in ovarian cancer (see Hough et al., Cancer Res. 61(10): 3869-3876, 2001 and Bagnoli et al., Oncogene, 19(41): 4754-4763, 2000). Finally, the specific pattern of caveolin (CAV1) under-expression and Folate receptor (FOLR1) over-expression disclosed herein (see Tables 4 & 5, Addendum) is consistent with the reciprocal regulation of the expression of genes in ovarian cancer (see Bagnoli et al., Oncogene, 19(41): 4754-4763, 2000).

For each of the above specifically enumerated genes, a survey of the Serial Analysis of Gene Expression (SAGE) database (available through the UniGene search engine on the National Center for Biotechnology Information website) revealed that the expression of these genes is limited to a relatively small number of tissues, including ovarian cancers and some other tumors, for instance pancreatic or breast. In addition, SLP1 and SPP1 are secreted proteins that may be detectable as a diagnostic marker in the serum of a subject.

### EXAMPLE 4

### Classification of a Tumor into BRCA1-Linked or BRCA2-Linked Tumor Class

This example describes how to classify a tumor into a BRCA1-like or BRCA2-like tumor type using compound covariate prediction analysis.

Class prediction can be performed using a Compound Covariate Predictor tool, available as part of the BRB Array Tools software provided for download on the National Cancer Institute Internet website. Detailed information about the Compound Covariate Predictor is provided by the Biometric Research Branch, National Cancer Institute and can be found in the following technical reports listed at that site” McShane et al., “Methods for assessing reproducibility of clustering patterns observed in analyses of microarray data” and Radmacher et al., “A paradigm for class prediction using gene expression profiles.”

The compound covariate predictor tool creates a multivariate predictor for one of two classes for each sample using markers in the multivariate predictor that are univariately significant at the selected significance cutoff for a given set of data (see discussion above in Section V. D, “Compound Covariate Predictor Analysis.”). The statistical significance cutoff for a given set of data can be chosen based upon the level of confidence desired.

By way of example, the markers provided in Table 10 satisfy a cutoff of P<0.0005, and are therefore suitable for use with compound covariate predictor analysis. The multivariate predictor is a weighted linear combination of log-ratios for genes that are univariately significant. The weight consists of the univariate t-statistics for comparing the classes.

Using the compound covariate predictor and the markers provided in Table 10, a sample of ovarian tissue can be classified into a BRCA1-like or BRCA2-like tumor. Samples are prepared as described in Example 1, and logarithmic expression ratios obtained for each marker used in the compound covariate predictor analysis.

The markers provided in Table 10 were used to segregate BRCA1-linked and BRCA1-type sporadic tumor samples from BRCA2-linked and BRCA2-type sporadic samples, in a multivariate analysis. Based upon the information regarding these classes that was obtained using other approaches (such as hierarchical clustering, see Example 1), compound covariate predictor analysis classified the tumors with 92% accuracy (see Table 11).

Using this method, an unknown tumor can be classified into one of any two groups provided that markers that are univariately significant at the selected significance cutoff for the desired groups are known. In addition, the gene expression data for the markers should be obtained using the same reference standard as the sample tumor.

Further analysis, such as a “leave-one-out” approach may be employed to check the veracity of the compound covariate predictor model. In this approach, each of the tumors is individually segregated, and the analysis completed using that tumor against the remaining samples. In this way, the strength of the data set is measured against each individual sample (tumor), confirming that the data set is useful, independently of any individual sample. See Radmacher et al., “A paradigm for class prediction using gene expression profiles,” available on the Biometric Research Branch, National Cancer Institute Internet site.

### EXAMPLE 5

### Expression of Ovarian Cancer-Related Polypeptides

This example describes how to express the ovarian cancer-related proteins disclosed herein using various techniques.

The disclosed ovarian cancer-related proteins (and fragments thereof) can be expressed by standard laboratory technique. After expression, the purified ovarian cancer-related protein or polypeptide may be used for instance for functional analyses, antibody production, diagnostics, prognostics, and patient therapy, e.g., for prevention or treatment of ovarian cancer. Furthermore, the DNA sequences encoding the disclosed ovarian cancer-related proteins can be manipulated in studies to understand the expression of these genes and the function of their products. Mutant forms of human ovarian cancer-related proteins (and corresponding encoding sequences) may be isolated based upon information contained herein, and may be studied in order to detect alteration in expression patterns in terms of relative quantities, tissue specificity and functional properties of the encoded mutant ovarian cancer-related protein. Partial or full-length cDNA sequences that encode the subject protein may be ligated into bacterial expression vectors. Methods for expressing large amounts of protein from a cloned gene introduced into Escherichia coli (E. coli) or other prokaryotes may be utilized for the purification, localization, and functional analysis of proteins. For example, fusion proteins consisting of amino terminal peptides encoded by a portion of the E. coli lacZ or trpE gene linked to an ovarian cancer-related protein may be used to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immunofluorescence.

Intact native protein may also be produced in E. coli in large amounts for functional studies. Methods and plasmid vectors for producing fusion proteins and intact native proteins in bacteria are described in Sambrook et al. (In Molecular Cloning: A Laboratory Manual, Ch. 17, CSHL, New York, 1989). Such fusion proteins may be made in large amounts, are easy to purify, and can be used to elicit antibody response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented in Sambrook et al. (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning: A Laboratory Manual, Ch. 17, CSHL. New York, 1989). Vector systems suitable for the expression of lacZ fusion genes include the pUR series of vectors (see Ruther and Muller-Hill, EMBO J. 2:1791, 1983), pEX 1-3 (see Stanley and Luzio, EMBO J. 3:1429, 1984) and pMR100 (see Gray et al., Proc. Natl. Acad. Sci. USA 79:6598, 1982). Vectors suitable for the production of intact native proteins include pKC30 (see Shimatake and Rosenberg, Nature 292:128, 1981), pKK177-3 (see Amann and Brositis, Gene 40:183, 1985) and pET-3 (see Studiar and Moffatt, J. Mol. Biol. 189:113, 1986). Fusion proteins, for instance fusions that incorporate a portion of an ovarian cancer-related protein, may be isolated from protein gels, lyophilized, ground into a powder and used as an antigen. The DNA sequence can also be transferred from its existing context to other cloning vehicles, such as other plasmids, bacteriophages, cosmids, animal viruses and yeast artificial chromosomes (YACs) (see Burke et al., Science 236:806-812, 1987). These vectors may then be introduced into a variety of hosts including somatic cells, and simple or complex organisms, such as bacteria, fungi (see Timberlake and Marshall, Science 244:1313-1317, 1989), invertebrates, plants (see Gasser and Fraley, Science 244:1293, 1989), and animals (see Pursel et al., Science 244:1281-1288, 1989), which cell or organisms are rendered transgenic by the introduction of the heterologous ovarian cancer-related cDNA.

For expression in mammalian cells, the cDNA sequence may be ligated to heterologous promoters, such as the simian virus (SV) 40 promoter in the pSV2 vector (see Mulligan and Berg, Proc. Natl. Acad. Sci. USA 78:2072-2076, 1981), and introduced into cells, such as monkey COS-1 cells (see Gluzman, Cell 23:175-182, 1981), to achieve transient or long-term expression. The stable integration of the chimeric gene construct may be maintained in mammalian cells by biochemical selection, for example with neomycin (see Southern and Berg, J. Mol. Appl. Genet. 1: 327-341, 1982) or mycophenolic acid (see Mulligan and Berg, Proc. Natl. Acad. Sci. USA 78: 2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR.

The cDNA sequence (or portions derived from it) or a mini gene (a cDNA with an intron and its own promoter) may be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (see Mulligan et al., Proc. Natl. Acad. Sci. USA 78:1078-2076, 1981; Gorman et al., Proc. Natl. Acad. Sci USA 78:6777-6781, 1982). The level of expression of the cDNA can be manipulated with this type of vector, either by using promoters that have different activities (for example, the baculovirus pAC373 can express cDNAs at high levels in S. frugiperda cells (see Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (see Lee et al., Nature 294:228, 1982). The expression of the cDNA can be monitored in the recipient cells 24 to 72 hours after introduction (transient expression).

In addition, some vectors contain selectable markers such as the gpt (see Mulligan and Berg, Proc. Natl. Acad. Sci. USA 78:2072-2076, 1981) or neo (see Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (see Sarver et al., Mol. Cell Biol. 1:486, 1981) or Epstein-Barr (see Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (see Alt et al., J. Biol. Chem. 253:1357, 1978).

The transfer of DNA into eukaryotic, in particular human or other mammalian cells, is now a conventional technique. The vectors are introduced into the recipient cells as pure DNA (transfection) by, for example, precipitation with calcium phosphate (see Graham and vander Eb, Virology 52:466, 1973) or strontium phosphate (see Brash et al., Mol. Cell Biol. 7:2013, 1987), electroporation (see Neumann et al., EMBO J. 1:841, 1982), lipofection (see Felgner et al., Proc. Natl. Acad. Sci USA 84:7413, 1987), DEAE dextran (see McCuthan et al., J. Natl. Cancer Inst. 41:351, 1968), microinjection (see Mueller et al., Cell 15:579, 1978), protoplast fusion (see Schafner, Proc. Natl. Acad. Sci. USA 77:2163-2167, 1980), or pellet guns (see Klein et al., Nature 327:70, 1987). Alternatively, the cDNA, or fragments thereof, can be introduced by infection with virus vectors. Systems are developed that use, for example, retroviruses (see Bernstein et al., Gen. Engr'g 7:235, 1985), adenoviruses (see Ahmad et al., J. Virol. 57:267, 1986), or Herpes virus (see Spaete et al., Cell 30:295, 1982). MB1 encoding sequences can also be delivered to target cells in vitro via non-infectious systems, for instance liposomes.

These eukaryotic expression systems can be used for studies of ovarian cancer-related nucleic acids (such as those listed in Table 1) and mutant forms of these molecules, as well as ovarian cancer-related proteins and mutant forms of these protein. Such uses include, for example, the identification of regulatory elements located in the 5′ region of ovarian cancer-related genes on genomic clones that can be isolated from human genomic DNA libraries. The eukaryotic expression systems may also be used to study the function of the normal ovarian cancer-related proteins, specific portions of these proteins, or of naturally occurring or artificially produced mutant versions of ovarian cancer-related proteins.

Using the above techniques, the expression vectors containing ovarian cancer-related gene sequence or cDNA, or fragments or variants or mutants thereof, can be introduced into human cells, mammalian cells from other species or non-mammalian cells as desired. The choice of cell is determined by the purpose of the treatment. For example, monkey COS cells (see Gluzman. Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

The present disclosure thus encompasses recombinant vectors that comprise all or part of an ovarian cancer-related gene or cDNA sequence (e.g., those listed in Table 1), for expression in a suitable host. In some embodiments, the ovarian cancer-related nucleic acid sequence is operatively linked in the vector to an expression control sequence to form a recombinant DNA molecule, so that the ovarian cancer-related polypeptide can be expressed. The expression control sequence may be selected from the group consisting of sequences that control the expression of genes of prokaryotic or eukaryotic cells and their viruses, and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors, and combinations thereof.

The host cell, which may be transfected with the vector of this disclosure, may be selected from the group consisting of E. coli, Pseudomonas, Bacillus subtilis, B. stearothermophilus or other bacilli; other bacteria; yeast; fungi; insect; mouse or other animal; or plant hosts; or human tissue cells.

It is appreciated that for mutant or variant ovarian cancer-related DNA sequences, similar systems are employed to express and produce the mutant product. In addition, fragments of an ovarian cancer-related protein can be expressed essentially as detailed above. Such fragments include individual ovarian cancer-related protein domains or sub-domains, as well as shorter fragments such as peptides. Ovarian cancer-related protein fragments (e.g., those having therapeutic properties) may be expressed in this manner also.

### EXAMPLE 6

### Suppression of Ovarian Cancer-Related Increased Gene Expression

This example describes how the ovarian cancer-related nucleic acids disclosed herein may be suppressed using various techniques.

A reduction of ovarian cancer-related protein expression in a transgenic cell may be obtained by introducing into cells an antisense construct based on an ovarian cancer-related protein encoding sequence, such as a cDNA or gene sequence or flanking regions thereof of any one of the proteins encoded by the nucleic acid molecules listed in Table 1, Table 9 or elsewhere herein. For antisense suppression, a nucleotide sequence encoding an ovarian cancer-related protein that is overexpressed in ovarian cancer, e.g. all or a portion of the small cell lung carcinoma cluster 4 antigen (CD24) (SEQ ID NO: 181-182), secretory leukocyte protease inhibitor antileukoproteinase (SLP1) (SEQ ID NO: 340-341), secreted phosphoprotein 1 (SPP1) (SEQ ID NO: 342), B-factor, properdin (BF) (SEQ ID NO: 343-344), “homolog of Cks1=p34Cdc28/Cdc2-associated protein” (CKS1) (SEQ ID NO: 345-347), matrix metalloproteinase 7 (MMP7) (SEQ ID NO: 348-349), paired box gene 8 (PAX8) (SEQ ID NO: 350-351), serine protease inhibitor, Kunitz type, 2 (SPINT2) (SEQ ID NO: 352-353), ZW10 interactor (ZWINT) (SEQ ID NO: 354), diacylglycerol kinase (DGKH) (SEQ ID NO: 355), high-mobility group (nonhistone chromosomal) protein isoforms I and Y (HMG1Y) (SEQ ID NO: 356), Syndecan-4-amphiglycan-ryudocan core protein (SDC4) (SEQ ID NO: 357-359), cyclin-dependent kinase inhibitor 2A (CDKN2A) (SEQ ID NO: 360), sodium channel, nonvoltage-gated 1 alpha (SCNN1A) (SEQ ID NO: 361-362), lactate dehydrogenase A (LDHA) (SEQ ID NO: 363), adult folate receptor (FOLR1) (SEQ ID NO: 364-365), Triosephosphate isomerase 1 (TP11) (SEQ ID NO: 366-367), kallikrein 8 (neuropsin/ovasin) (KLK8) (SEQ ID NO: 368), CXC chemokine receptor 4-fusin-neuropeptide Y receptor-L3 (CXCR4) (SEQ ID NO: 200), kinesin-like 1 (KNSL1) (SEQ ID NO: 369-370), H2A histone family, member O (H2AFO) (SEQ ID NO: 371-372), major histocompatibility complex, class II, DR alpha, HLA-DRA, cysteine-rich protein 1 (intestinal) (CR1P1) (SEQ ID NO: 375), pyrophosphatase (inorganic), (PP) (SEQ ID NO: 376), EST 666391, glucose transporter (HepG2) (SLC2A1) (SEQ ID NO: 379-381), EST 897770, hepatoma-derived growth factor (HDGF) (SEQ ID NO: 383-385), argininosuccinate synthetase (ASS) (SEQ ID NO: 386), claudin 4 (CLDN4) (SEQ ID NO: 387-388), preferentially expressed antigen in melanoma (PRAME) (SEQ ID NO: 389), LAR=LCA-homologue (PTPRF) (SEQ ID NO: 390-391), eyes absent (Drosophila) homolog 2 (EYA2) (SEQ ID NO: 392-393), L-myc (MYCL1) (SEQ ID NO: 394-396), STAT1=IFN alpha/beta-responsive transcription factor ISGF3 beta subunits (p91/p84) (STAT1) (SEQ ID NO: 397-399), mitochondrial carrier homolog 2 (MTCH2) (SEQ ID NO: 400-401), 5-hydroxytryptamine (serotonin) receptor 3A (HTR3A) (SEQ ID NO: 402), cyclin E1 (CCNE1) (SEQ ID NO: 403-404), cadherin 6, type 2, K-cadherin (fetal kidney) (CDH6) (SEQ ID NO: 405), 5′-AMP-activated protein kinase-gamma-1 subunit (PRKA4G) (SEQ ID NO: 406408), defensin beta 1 (DEFB1) (SEQ ID NO: 409), actin related protein ⅔ complex, subunit 1A (41 kD) (ARPC1B) (SEQ ID NO: 410-411), PKC iota=Protein kinase C, iota (PRKC1) (SEQ ID NO: 412-414), glyceraldehyde-3-phosphate dehydrogenase (GAPD) (SEQ ID NO: 415), complement component 2 (C2) (SEQ ID NO: 416-417), H2A histone family, member Y (H2AFY) (SEQ ID NO: 418-419), transmembrane 4 superfamily member 1 (TM4SF1) (SEQ ID NO: 420-421), glyceraldehyde-3-phosphate dehydrogenase (GAPD) (SEQ ID NO: 422-423), Interferon-inducible protein 1-8U (IFITM3) (SEQ ID NO: 424-426), glycine dehydrogenase (decarboxylating; glycine decarboxylase, glycine cleavage system protein P) (GLDC) (SEQ ID NO: 427-428), calumenin (CALU) (SEQ ID NO: 429-430), hemoglobin alpha 2 (HBA2) (SEQ ID NO: 431-432), S100 calcium-binding protein A11 (calgizzarin) (S100A11) (SEQ ID NO: 433), Lactate dehydrogenase A (LDHA) (SEQ ID NO: 434-436), ubiquitin-conjugating enzyme E2C (UBE2C) (SEQ ID NO: 437), E2F-3=pRB-binding transcription factor=KIAA0075 (E2F3) (SEQ ID NO: 438-440), E-cadherin (CDH1) (SEQ ID NO: 441-442), proteasome (prosome, macropain) activator subunit 2 (PA28 beta) (PSME2) (SEQ ID NO: 443444), OP-1=osteogenic protein in the TGF-beta family (BMP7) (SEQ ID NO: 445-447), and topoisomerase II (TOP2A) (SEQ ID NO: 448) cDNA or gene, is arranged in reverse orientation relative to the promoter sequence in the transformation vector. Other aspects of the vector may be chosen as for any other expression vector (see, e.g., Example 4).

The introduced sequence need not be a full-length human ovarian cancer-related cDNA or gene, and need not be exactly homologous to the equivalent sequence found in the cell type to be transformed. Generally, however, where the introduced sequence is of shorter length, a higher degree of homology to the ovarian cancer-related sequence likely will be needed for effective antisense suppression. The introduced antisense sequence in the vector may be at least thirty nucleotides in length, and improved antisense suppression will typically be observed as the length of the antisense sequence increases. The length of the antisense sequence in the vector advantageously may be greater than 100 nucleotides.

Although the exact mechanism by which antisense RNA molecules interfere with gene expression has not been elucidated, it is believed that antisense RNA molecules bind to the endogenous mRNA molecules and thereby inhibit translation of the endogenous mRNA.

Suppression of endogenous ovarian cancer-related gene expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

In addition, dominant negative mutant forms of the disclosed ovarian cancer-related sequences may be used to block endogenous activity of the corresponding gene products.

Suppression can also be achieved using small inhibitory RNA molecules (siRNAs) (see, for instance, Caplen et al., Proc. Natl. Acad. Sci. 98(17): 9742-9747, 2001, and Elbashir et al., Nature 411: 494-498, 2001). Thus, this disclosure also encompasses siRNAs that correspond to an ovarian cancer-related nucleic acid, which siRNA is capable of suppressing the expression or function of its cognate (target) ovarian cancer-related protein. Also encompassed are methods of suppressing the expression or activity of an ovarian cancer-related molecule using an siRNA.

Suppression of expression of an ovarian cancer-related gene can be used, for instance, to treat, reduce, or prevent cell proliferative and other disorders caused by over-expression or unregulated expression of the corresponding ovarian cancer-related gene. In particular, suppression of expression of sequences disclosed herein as being up-regulated in ovarian cancer can be used to treat, reduce, or prevent progression to a later stage of ovarian cancer.

### EXAMPLE 7

### Nucleic Acid-Based Analysis

This example describes how to use the ovarian cancer-related nucleic acids disclosed herein to detect and analyze neoplasms and mutations in ovarian cancer-related nucleic acids that may result in neoplasms.

The ovarian cancer-related nucleic acid molecules provided herein, and combinations of these molecules, can be used in methods of genetic testing for neoplasms (e.g., ovarian or other cancers) or predisposition to neoplasms owing to altered expression of ovarian cancer-related nucleic acid molecules (e.g., deletion, genomic amplification or mutation, or over- or under-expression in comparison to a control or baseline). For such procedures, a biological sample of the subject, which biological sample contains either DNA or RNA derived from the subject, is assayed for a mutated, amplified or deleted ovarian cancer-related nucleic acid molecule, or for over- or under-expression of an ovarian cancer-related nucleic acid molecule. Suitable biological samples include samples containing genomic DNA or RNA (including mRNA), obtained from body cells of a subject, such as those present in peripheral blood, urine, saliva, tissue biopsy, surgical specimen, amniocentesis samples and autopsy material.

The detection in the biological sample of a mutant ovarian cancer-related nucleic acid molecule, a mutant ovarian cancer-related RNA, an amplified or homozygously or heterozygously deleted ovarian cancer-related nucleic acid molecule, or over- or under-expression of an ovarian cancer-related nucleic acid molecule, may be performed by a number of methodologies, examples of which are provided.

A. Detection of Unknown Mutations:

Unknown mutations in ovarian cancer-related nucleic acid molecules can be identified through polymerase chain reaction amplification of reverse transcribed RNA (RT-PCR) or DNA isolated from breast or ovary or other tissue, followed by direct DNA sequence determination of the products; single-strand conformational polymorphism analysis (SSCP) (for instance, see Hongyo et al., Nucleic Acids Res. 21:3637-3642, 1993); chemical cleavage (including HOT cleavage) (Bateman et al., Am. J. Med. Genet. 45:233-240, 1993; reviewed in Ellis et al., Hum. Mutat. 11:345-353, 1998); denaturing gradient gel electrophoresis (DGGE); ligation amplification mismatch protection (LAMP); or enzymatic mutation scanning (Taylor and Deeble, Genet. Anal. 14:181-186, 1999), followed by direct sequencing of amplicons with putative sequence variations.

B. Detection of Known Mutations:

The detection of specific known DNA mutations in ovarian cancer-related nucleic acid molecules may be achieved by methods such as hybridization using allele specific oligonucleotides (ASOs) (see Wallace et al., CSHL Symp. Quant. Biol. 51:257-261, 1986), direct DNA sequencing (see Church and Gilbert. Proc. Natl. Acad. Sci. USA 81:1991-1995, 1988), the use of restriction enzymes (see Flavell et al., Cell 15:25, 1978; Geever et al., Proc. Natl. Acad. Sci. U.S.A. 8(8): 5081-5085, 1981), discrimination on the basis of electrophoretic mobility in gels with denaturing reagent (see Myers and Maniatis, Cold Spring Harbor Symp. Quant. Biol. 51:275-284, 1986), RNase protection (see Myers et al., Science 230:1242, 1985), chemical cleavage (see Cotton et al., Proc. Nail. Acad. Sci. USA 85:4397-4401, 1985), and the ligase-mediated detection procedure (see Landegren et al., Science 241:1077-1080, 1988). Oligonucleotides specific to normal or mutant MB1 sequences can be chemically synthesized using commercially available machines. These oligonucleotides can then be labeled radioactively with isotopes (such as ³²P) or non-radioactively, with tags such as biotin (see Ward and Langer et al., Proc. Nail. Acad. Sci. USA 78:6633-6657, 1981), and hybridized to individual DNA samples immobilized on membranes or other solid supports by dot-blot or transfer from gels after electrophoresis. These specific sequences are visualized by methods such as autoradiography or fluorometric (see Landegren et al., Science 242:229-237, 1989) or colorimetric reactions (see Gebeyehu et al., Nucleic Acids Res. 15:4513-4534, 1987). Using an ASO specific for a normal allele, the absence of hybridization would indicate a mutation in the particular region of the gene, or deleted MB I gene. In contrast, if an ASO specific for a mutant allele hybridizes to a clinical sample then that would indicate the presence of a mutation in the region defined by the ASO.

C. Detection of Genomic Amplification or Deletion

Gene dosage (copy number) can be important in neoplasms; it is therefore advantageous to determine the number of copies of ovarian cancer-related nucleic acids in biological samples of a subject, e.g., serum or ovary samples. Probes generated from the disclosed encoding sequence of in ovarian cancer-related nucleic acid molecules can be used to investigate and measure genomic dosage of the corresponding ovarian cancer-related genomic sequence.

Appropriate techniques for measuring gene dosage are known in the art; see for instance, U.S. Pat. No. 5,569,753 (“Cancer Detection Probes”) and Pinkel et al. (Nat. Genet. 20:207-211, 1998) (“High Resolution Analysis of DNA Copy Number Variation using Comparative Genomic Hybridization to Microarrays”).

Determination of gene copy number in cells of a patient-derived sample using other techniques is known in the art. For example, amplification of an ovarian cancer-related nucleic acid sequence in cancer-derived cell lines as well as uncultured ovarian cancer or other cells can be carried out using bicolor FISH analysis. By way of example, interphase FISH analysis of breast cancer cell lines can be carried out as previously described (see Barlund et al., Genes Chromo. Cancer 20:372-376, 1997). The hybridizations can be evaluated using a Zeiss fluorescence microscope.

For tissue microarrays, the FISH can be performed as described in Kononen et al. (Nat. Med. 4:844-847, 1998). Briefly, consecutive sections of the array are deparaffinized, dehydrated in ethanol, denatured at 74° C. for 5 minutes in 70% formamide/2×SSC, and hybridized with test and reference probes. The specimens containing tight clusters of signals or >3-fold increase in the number of test probe as compared to chromosome 17 centromere in at least 10% of the tumor cells may be considered as amplified. Microarrays can be constructed as described in WO 99/44063A2 and WO 99/44062A1.

C. Detection of mRNA Expression Levels

Altered expression of an ovarian cancer-related molecule also can be detected by measuring the cellular level of ovarian cancer-related nucleic acid molecule-specific mRNA. mRNA can be measured using techniques well known in the art, including for instance Northern analysis, RT-PCR and mRNA in situ hybridization. Details of mRNA analysis procedures can be found, for instance, in Example 1, Example 3, and Sambrook et al. (ed.), Molecular Cloning. A Laboratory Manual, 2nd ed., vol. 1-3, Cold Spring Harbor Laboratory Press, Cold Spring Harbor, N.Y., 1989.

The nucleic acid-based diagnostic methods of this disclosure are predictive of ovarian cancer. Cells of any tumors that demonstrate altered expression levels (e.g., through genomic amplification, deletion, mutation, or other over- or under-expression) of nucleotide sequences that share homology with the ovarian cancer-related nucleic acids disclosed herein are aggressive tumor cells, and result in decreased survival, increased metastasis, increased rates of clinical, and overall worsened prognosis.

### EXAMPLE 8

### Production of Protein Specific Binding Agents

This example describes how to use the ovarian cancer-related molecules disclosed herein to produce binding agents useful in preventing ovarian cancer.

Monoclonal or polyclonal antibodies may be produced to any of the disclosed ovarian cancer-related proteins, or mutant forms of these proteins. Optimally, antibodies raised against these proteins, or peptides from within such proteins, would specifically detect the protein or peptide with which the antibodies are generated. That is, an antibody generated to the BMP7 protein or another specified protein (see Table 1) or a fragment thereof would recognize and bind that protein and would not substantially recognize or bind to other proteins found in human cells.

The determination that an antibody specifically detects a designated protein (e.g., an ovarian cancer-related protein as disclosed herein) can be made by any one of a number of standard immunoassay methods; for instance, the Western blotting technique (see Sambrook et al., In Molecular Cloning: A Laboratory Manual, CSHL, New York, 1989). To determine that a given antibody preparation (such as one produced in a mouse) specifically detects a designated protein by Western blotting, total cellular proteins are extracted from cells (for example, human ovary) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase. Application of an alkaline phosphatase substrate 5-bromo-4-chloro-3-indolyl phosphate/nitro blue tetrazolium results in the production of a dense blue compound by immunolocalized alkaline phosphatase. Antibodies that specifically detect the designated protein will, by this technique, be shown to bind to the designated protein band (which will be localized at a given position on the gel determined by its molecular weight). Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody-protein binding.

Substantially pure ovarian cancer-related protein or protein fragment (peptide) suitable for use as an immunogen may be isolated from transfected or transformed cells, as described above. Concentration of protein or peptide in the final preparation is adjusted, for example, by concentration on an Amicon filter device, to the level of a few micrograms per milliliter. Monoclonal or polyclonal antibody to the protein can then be prepared as follows:

A. Monoclonal Antibody Production by Hybridoma Fusion

Monoclonal antibody to epitopes of a designated protein (such as an ovarian cancer-related protein, including one encoded by a nucleic acid listed in Table 1) identified and isolated as described can be prepared from murine hybridomas according to the classical method of Kohler and Milstein (Nature 256:495-497, 1975) or derivative methods thereof Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Meth. Enzymol. 70: 419-439, 1980), and derivative methods thereof. Selected positive clones can be expanded and their monoclonal antibody product harvested for use. Detailed procedures for monoclonal antibody production are described in Harlow and Lane (Antibodies, A Laboratory Manual, CSHL, New York, 1988).

B. Polyclonal Antibody Production by Immunization

Polyclonal antiserum containing antibodies to heterogeneous epitopes of a single protein can be prepared by immunizing suitable animals with the expressed protein, which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with either inadequate or excessive doses of antigen resulting in low titer antisera. Small doses (ng level) of antigen administered at multiple intradermal sites appear to be most reliable. An effective immunization protocol for rabbits can be found in Vaitukaitis et al. (J. Clin. Endocrinol. Metab. 33: 988-991, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In Handbook of Experimental Immunology, Wier (ed.) Chapter 19. Blackwell, 1973). Plateau concentration of antibody is usually in the range of about 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

C. Antibodies Raised against Synthetic Peptides

A third approach to raising antibodies against the subject ovarian cancer-related proteins or peptides is to use one or more synthetic peptides synthesized on a commercially available peptide synthesizer based upon the predicted amino acid sequence of the desired ovarian cancer-related protein or peptide.

D. Antibodies Raised by Injection of Protein Encoding Sequence

Antibodies also may be raised against proteins and peptides related to ovarian cancer as described herein by subcutaneous injection of a DNA vector that expresses the desired ovarian cancer-related protein, or a fragment thereof, into laboratory animals, such as mice. Delivery of the recombinant vector into the animals may be achieved using a hand-held form of the Biolistic system (see Sanford et al., Particulate Sci. Technol. 5:27-37, 1987) as described by Tang et al. (Nature 356:152-154, 1992). Expression vectors suitable for this purpose may include those that express the ovarian cancer-related sequence under the transcriptional control of either the human β-actin promoter or the cytomegalovirus (CMV) promoter.

Antibody preparations prepared according to these protocols are useful in quantitative immunoassays that determine concentrations of antigen-bearing substances in biological samples; they also can be used semi-quantitatively or qualitatively to identify the presence of antigen in a biological sample; or for immunolocalization of the corresponding ovarian cancer-related protein.

For administration to human patients, antibodies, e.g., ovarian cancer-related protein specific monoclonal antibodies (such as antibodies to the proteins encoded by the encoding sequences listed to in Table 1), can be humanized by methods known in the art. Antibodies with a desired binding specificity can be commercially humanized (Scotgene, Scotland, UK; Oxford Molecular, Palo Alto, Calif.). Alternatively, human antibodies can be produced. Methods for producing human antibodies are known in the art; see, for instance, Canevari et al., Int. J. Biol. Markers 8:147-150, 1993 and Green, J. Immunol. Meth. 231:11-23, 1999, for instance.

### EXAMPLE 9

### Protein-Based Analysis

This example describes how to use the ovarian cancer-related molecules disclosed herein to quantitate the level of one or more ovarian cancer-related proteins in a subject.

An alternative method of diagnosing, staging, detecting, or predicting ovarian cancer is to quantitate the level of one or more ovarian cancer-related proteins in a subject, for instance in the cells of the subject. This diagnostic tool is useful for detecting reduced or increased levels of ovarian cancer-related proteins. Localization and/or coordinated expression (temporally or spatially) of ovarian cancer-related proteins can also be examined using well known techniques. The determination of reduced or increased ovarian cancer-related protein levels, in comparison to such expression in a normal subject (e.g., a subject not having ovarian cancer or not having a predisposition developing this condition, disease or disorder, would be an alternative or supplemental approach to the direct determination of ovarian cancer-related nucleic acid levels by the methods outlined above and equivalents. The availability of antibodies specific to specific ovarian cancer-related protein(s) will facilitate the detection and quantitation of cellular ovarian cancer-related protein(s) by one of a number of immunoassay methods which are well known in the art and are presented in Harlow and Lane (Antibodies, A Laboratory Manual, CSHL, New York, 1988). Methods of constructing such antibodies are discussed above, in Example 7.

Any standard immunoassay format (e.g., ELISA, Western blot, or RIA assay) can be used to measure altered expression of ovarian cancer-related polypeptide or protein levels; comparison is to wild-type (normal) ovarian cancer-related protein levels, and a difference in ovarian cancer-related polypeptide levels is indicative of a biological condition resulting from altered expression of ovarian cancer-related polypeptides or proteins, such as neoplasia. Whether the key difference is an increase or a decrease is dependent on the specific ovarian cancer-related protein under examination, as discussed herein. Immunohistochemical techniques may also be utilized for ovarian cancer-related polypeptide or protein detection and quantification. For example, a tissue sample may be obtained from a subject, and a section stained for the presence of an ovarian cancer-related protein using the appropriate ovarian cancer-related protein specific binding agent and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating an ovarian cancer-related protein, a biological sample of the subject, which sample includes cellular proteins, is required. Such a biological sample may be obtained from body cells, such as those present in peripheral blood, urine, saliva, tissue biopsy, amniocentesis samples, surgical specimens and autopsy material, particularly breast cells. Quantitation of an ovarian cancer-related protein can be achieved by immunoassay and the amount compared to levels of the protein found in healthy cells. A significant difference (either increase or decrease) in the amount of ovarian cancer-related protein in the cells of a subject compared to the amount of the same ovarian cancer-related protein found in normal human cells is usually about a 10% or greater change, for instance 20%, 30%, 40%, 50% or greater difference. Substantial under- or over-expression of one or more ovarian cancer-related protein(s), may be indicative of neoplasia or a predilection to neoplasia or metastasis, and especially ovarian epithelial cancer.

The protein-based diagnostic methods as described herein are predictive of ovarian cancer. Cells of any tumors that demonstrate altered expression levels (e.g., through genomic amplification, deletion, mutation, or other over- or under-expression) of nucleotide sequences that share homology with the ovarian cancer-related nucleic acids disclosed herein are aggressive tumor cells, and result in decreased survival, increased metastasis, increased rates of clinical recurrence, and overall worsened prognosis.

### EXAMPLE 10

### Gene Therapy

This example describes how to use the ovarian cancer-related molecules and analysis methods disclosed herein to effect gene therapy for the treatment of ovarian cancer.

Gene therapy approaches for combating neoplasia (particularly ovarian cancer) in subjects are made possible by the present disclosure.

Retroviruses have been considered a preferred vector for experiments in gene therapy, with a high efficiency of infection and stable integration and expression (see Orkin et al., Prog. Med. Genet. 7:130-142, 1988). A full-length ovarian cancer-related gene or cDNA can be cloned into a retroviral vector and driven from either its endogenous promoter or from the retroviral LTR (long terminal repeat). Other viral transfection systems may also be utilized for this type of approach, including adenovirus, adeno-associated virus (AAV) (see McLaughlin et al., J. Virol. 62:1963-1973, 1988), Vaccinia virus (Moss et al., Annu. Rev. Immunol. 5:305-324, 1987), Bovine Papilloma virus (Rasmussen et al., Methods Enzymol. 139:642-654, 1987) or members of the herpesvirus group such as Epstein-Barr virus (Margolskee et al., Mol. Cell. Biol. 8:2837-2847, 1988).

Developments in gene therapy techniques include the use of RNA-DNA hybrid oligonucleotides, as described by Cole-Strauss et al. (Science 273:1386-1389, 1996). This technique may allow for site-specific integration of cloned sequences, thereby permitting accurately targeted gene replacement.

In addition to delivery of ovarian cancer-related protein encoding sequences to cells using viral vectors, it is possible to use non-infectious methods of delivery. For instance, lipidic and liposome-mediated gene delivery has recently been used successfully for transfection with various genes (for reviews, see Templeton and Lasic, Mol. Biotechnol. 11:175-180, 1999; Lee and Huang, Crit. Rev. Ther. Drug Carrier Syst. 14:173-206; and Cooper, Semin. Oncol. 23:172-187, 1996). For instance, cationic liposomes have been analyzed for their ability to transfect monocytic leukemia cells, and shown to be a viable alternative to using viral vectors (de Lima et al., Mol. Membr. Biol. 16:103-109, 1999). Such cationic liposomes can also be targeted to specific cells through the inclusion of, for instance, monoclonal antibodies or other appropriate targeting ligands (see Kao et al., Cancer Gene Ther. 3:250-256, 1996).

To reduce the level of ovarian cancer-related gene expression, gene therapy can be carried out using antisense or other suppressive constructs, the construction of which is discussed above (Example 4).

### EXAMPLE 11

### Kits

This example describes various kits for using the ovarian cancer-related molecules and analysis methods disclosed herein.

Kits are provided to determine the level (or relative level) of expression of one or more species of ovarian cancer-related nucleic acids (e.g., mRNA) or one or more ovarian cancer-related protein (i.e., kits containing nucleic acid probes or antibodies or other ovarian cancer-related protein specific binding agents). Kits are also provided that contain the necessary reagents for determining gene copy number (genomic amplification or deletion), such as probes or primers specific for an ovarian cancer-related nucleic acid sequence. These kits can each include instructions, for instance instructions that provide calibration curves or charts to compare with the determined (e.g., experimentally measured) values.

A. Kits for Detection of Ovarian Cancer-Related Genomic Amplification or Deletion

The nucleotide sequence of ovarian cancer-related nucleic acid molecules disclosed herein, and fragments thereof, can be supplied in the form of a kit for use in detection of ovarian cancer-related genomic amplification/deletion and/or diagnosis of progression to or predilection to progress to ovarian epithelial cancer. In such a kit, an appropriate amount of one or more oligonucleotide primer specific for an ovarian cancer-related-sequence is provided in one or more containers. The oligonucleotide primers may be provided suspended in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers may be provided in pre-measured single use amounts in individual, typically disposable, tubes, or equivalent containers. With such an arrangement, the sample to be tested for the presence of ovarian cancer-related genomic amplification/deletion can be added to the individual tubes and in vitro amplification carried out directly.

The amount of each oligonucleotide primer supplied in the kit can be any amount, depending for instance on the market to which the product is directed. For instance, if the kit is adapted for research or clinical use, the amount of each oligonucleotide primer provided likely would be an amount sufficient to prime several in vitro amplification reactions. Those of ordinary skill in the art know the amount of oligonucleotide primer that is appropriate for use in a single amplification reaction. General guidelines may for instance be found in Innis et al. (PCR Protocols, A Guide to Methods and Applications, Academic Press, Inc., San Diego, Calif., 1990), Sambrook et al. (In Molecular Cloning: A Laboratory, Manual, Cold Spring Harbor, N.Y., 1989), and Ausubel et al. (In Current Protocols in Molecular Biology, John Wiley & Sons, New York, 1998).

A kit may include more than two primers, in order to facilitate the in vitro amplification of ovarian cancer-related genomic sequences (or a protein of such a sequence), for instance an ovarian cancer-related nucleic acid listed in Table 1, or the 5′ or 3′ flanking region thereof.

In some embodiments, kits may also include the reagents necessary to carry out in vitro amplification reactions, including, for instance, DNA sample preparation reagents, appropriate buffers (e.g., polymerase buffer), salts (e.g., magnesium chloride), and deoxyribonucleotides (dNTPs). Written instructions may also be included.

Kits may in addition include either labeled or unlabeled oligonucleotide probes for use in detection of the in vitro amplified sequences. The appropriate sequences for such a probe will be any sequence that falls between the annealing sites of two provided oligonucleotide primers, such that the sequence the probe is complementary to is amplified during the in vitro amplification reaction (if it is present in the sample).

It may also be advantageous to provided in the kit one or more control sequences for use in the in vitro amplification reactions. The design of appropriate positive control sequences is well known to one of ordinary skill in the appropriate art.

B. Kits for Detection of mRNA Expression

Kits similar to those disclosed above for the detection of ovarian cancer-related genomic amplification/deletion can be used to detect ovarian cancer-related mRNA expression levels (including over- or under-expression, in comparison to the expression level in a control sample). Such kits include an appropriate amount of one or more of the oligonucleotide primers for use in, for instance, reverse transcription PCR reactions, similarly to those provided above, with art-obvious modifications for use with RNA.

In some embodiments, kits for detection of ovarian cancer-related mRNA expression may also include reagents necessary to carry out RT-PCR or other in vitro amplification reactions, including, for instance, RNA sample preparation reagents (including e.g., an RNAse inhibitor), appropriate buffers (e.g., polymerase buffer), salts (e.g., magnesium chloride), and deoxyribonucleotides (dNTPs). Written instructions may also be included.

Kits may in addition include either labeled or unlabeled oligonucleotide probes for use in detection of an in vitro amplified target sequence. The appropriate sequences for such a probe will be any sequence that falls between the annealing sites of the two provided oligonucleotide primers, such that the sequence the probe is complementary to is amplified during the PCR reaction.

It may also be advantageous to provided in the kit one or more control sequences for use in the in vitro amplification reactions. The design of appropriate positive control sequences is well known to one of ordinary skill in the appropriate art.

Alternatively, kits may be provided with the necessary reagents to carry out quantitative or semi-quantitative Northern analysis of ovarian cancer-related mRNA. Such kits include, for instance, at least one ovarian cancer-related sequence-specific oligonucleotide for use as a probe. This oligonucleotide may be labeled in any conventional way, including with a selected radioactive isotope, enzyme substrate, co-factor, ligand, chemiluminescent or fluorescent agent, hapten, or enzyme.

C. Kits for Detection of Ovarian Cancer-Linked Protein or Peptide Expression

Kits for the detection of ovarian cancer-linked protein expression, for instance altered (over or under) expression of a protein encoded for by a nucleic acid molecule listed in Table 1 or elsewhere, are also encompassed herein. Such kits may include for example at least one target (ovarian cancer-linked) protein (e.g., all or a portion of the small cell lung carcinoma cluster 4 antigen (CD24) (SEQ ID NO: 181-182), secretory leukocyte protease inhibitor antileukoproteinase (SLP1) (SEQ ID NO: 340-341), secreted phosphoprotein 1 (SPP1) (SEQ ID NO: 342), B-factor, properdin (BF) (SEQ ID NO: 343-344), “homolog of Cks1=p34Cdc28/Cdc2-associated protein” (CKS1) (SEQ ID NO: 345-347), matrix metalloproteinase 7 (MMP7) (SEQ ID NO: 348-349), paired box gene 8 (PAX8) (SEQ ID NO: 350-351), serine protease inhibitor, Kunitz type, 2 (SPINT2) (SEQ ID NO: 352-353), ZW10 interactor (ZWINT) (SEQ ID NO: 354), diacylglycerol kinase (DGKH) (SEQ ID NO: 355), high-mobility group (nonhistone chromosomal) protein isoforms I and Y (HMG1Y) (SEQ ID NO: 356), Syndecan-4-amphiglycan-ryudocan core protein (SDC4) (SEQ ID NO: 357-359), cyclin-dependent kinase inhibitor 2A (CDKN2A) (SEQ ID NO: 360), sodium channel, nonvoltage-gated 1 alpha (SCNN1A) (SEQ ID NO: 361-362), lactate dehydrogenase A (LDHA) (SEQ ID NO: 363), adult folate receptor (FOLR1) (SEQ ID NO: 364-365), Triosephosphate isomerase 1 (TP11) (SEQ ID NO: 366-367), kallikrein 8 (neuropsin/ovasin) (KLK8) (SEQ ID NO: 368), CXC chemokine receptor 4-fusin-neuropeptide Y receptor-L3 (CXCR4) (SEQ ID NO: 200), kinesin-like 1 (KNSL1) (SEQ ID NO: 369-370), H2A histone family, member O (H2AFO) (SEQ ID NO: 371-372), major histocompatibility complex, class II, DR alpha, HLA-DRA, cysteine-rich protein 1 (intestinal) (CR1P1) (SEQ ID NO: 375), pyrophosphatase (inorganic), (PP) (SEQ ID NO: 376), EST (SEQ ID NO: 377-378) 666391, glucose transporter (HepG2), (SLC2A1) (SEQ ID NO: 379-381), EST (SEQ ID NO: 377-378) 897770, hepatoma-derived growth factor (HDGF) (SEQ ID NO: 383-385), argininosuccinate synthetase (ASS) (SEQ ID NO: 386), claudin 4 (CLDN4) (SEQ ID NO: 387-388), preferentially expressed antigen in melanoma (PRAME) (SEQ ID NO: 389), LAR=LCA-homologue (PTPRF) (SEQ ID NO: 390-391), eyes absent (Drosophila) homolog 2 (EYA2) (SEQ ID NO: 392-392), L-myc (MYCL1) (SEQ ID NO: 394-396), STAT1=IFN alpha/beta-responsive transcription factor ISGF3 beta subunits (p91/p84) (STAT1) (SEQ ID NO: 397-399), mitochondrial carrier homolog 2 (MTCH2) (SEQ ID NO: 400-401), 5-hydroxytryptamine (serotonin) receptor 3A (H7R3A (SEQ ID NO: 402), cyclin E1 (CCNE1) (SEQ ID NO: 403-404), cadherin 6, type 2, K-cadherin (fetal kidney) (CDH6) (SEQ ID NO: 405),5′-AMP-activated protein kinase-gamma-1 subunit (PRKAG1) (SEQ ID NO: 406-408), defensin beta 1 (DEFB1) (SEQ ID NO: 409), actin related protein ⅔ complex, subunit 1A (41 kD) (ARPC1B) (SEQ ID NO: 410-411), PKC iota=Protein kinase C, iota (PRKC1) (SEQ ID NO: 412-414), glyceraldehyde-3-phosphate dehydrogenase (GAPD) (SEQ ID NO: 415), complement component 2 (C2) (SEQ ID NO: 416-417), H2A histone family, member Y (H2AFY) (SEQ ID NO: 418-419), transmembrane 4 superfamily member 1 (TM4SF1) (SEQ ID NO: 420-421), glyceraldehyde-3-phosphate dehydrogenase (GAPD) (SEQ ID NO: 422-423), Interferon-inducible protein 1-8U (IFITM3) (SEQ ID NO: 424-426), glycine dehydrogenase (decarboxylating; glycine decarboxylase, glycine cleavage system protein P) (GLDC) (SEQ ID NO: 427-428), calumenin (CALU) (SEQ ID NO: 429-430), hemoglobin alpha 2 (HBA2) (SEQ ID NO: 431-432), S100 calcium-binding protein A11 (calgizzarin) (S100A11) (SEQ ID NO: 433), Lactate dehydrogenase A (LDHA) (SEQ ID NO: 434-436), ubiquitin-conjugating enzyme E2C (UBE2C) (SEQ ID NO: 437), E2F-3=pRB-binding transcription factor=KIAA0075 (E2F3) (SEQ ID NO: 438-440), E-cadherin (CDH1) (SEQ ID NO: 441-442), proteasome (prosome, macropain) activator subunit 2 (PA28 beta) (PSME2) (SEQ ID NO: 443-444), OP-1=osteogenic protein in the TGF-beta family (BMP7) (SEQ ID NO: 445-447), or topoisomerase II (TOP2A) (SEQ ID NO: 448) specific binding agent (e.g., a polyclonal or monoclonal antibody or antibody fragment), and may include at least one control. The ovarian cancer-linked protein specific binding agent and control may be contained in separate containers. The kits may also include a means for detecting ovarian cancer-related protein:agent complexes, for instance the agent may be detectably labeled. If the detectable agent is not labeled, it may be detected by second antibodies or protein A, for example, either of both of which also may be provided in some kits in one or more separate containers. Such techniques are well known.

Additional components in some kits include instructions for carrying out the assay. Instructions will allow the tester to determine whether ovarian cancer-linked expression levels are elevated or reduced in comparison to a control sample. Reaction vessels and auxiliary reagents such as chromogens, buffers, enzymes, etc. also may be included in the kits.

### EXAMPLE 12

### Identification of Therapeutic Compounds

This example describes how to use the ovarian cancer-related molecules disclosed herein to identify compounds for potential therapeutic use in treating, reducing, or preventing ovarian cancer or development or progression of ovarian cancer.

The ovarian cancer-related molecules disclosed herein, and more particularly the linkage of these molecules to cancer, can be used to identify compounds that are useful in treating, reducing, or preventing ovarian cancer or development or progression of ovarian cancer. These molecules can be used alone or in combination, for instance in sets of two or more that are linked to cancer or cancer progression.

By way of example, a test compound is applied to a cell, for instance a test cell, and at least one ovarian cancer-related molecule level and/or activity in the cell is measured and compared to the equivalent measurement from a test cell (or from the same cell prior to application of the test compound). If application of the compound alters the level and/or activity of an ovarian cancer-related molecule (for instance by increasing or decreasing that level), then that compound is selected as a likely candidate for further characterization. In particular examples, a test agent that opposes or inhibits an ovarian cancer-related change is selected for further study, for example by exposing the agent to an ovarian epithelial cancer cell in vitro, to determine whether in vitro growth is inhibited. Such identified compounds may be useful in treating, reducing, or preventing ovarian cancer or development or progression of ovarian cancer. In particular embodiments, the compound isolated will inhibit or inactivate an ovarian cancer-related molecule, for instance those represented by the nucleic acids listed in Table 1.

Methods for identifying such compounds optionally can include the generation of an ovarian cancer-related gene expression profile, as described herein. Control gene expression profiles useful for comparison in such methods may be constructed from normal ovarian tissue, including primary ovarian cancer tissue.

### EXAMPLE 12

### Gene Expression Profiles (Fingerprints)

This example describes how to use the ovarian cancer-related nucleic acids and analysis methods disclosed herein to generate and use gene expression profiles, or “fingerprints.”

With the provision herein of methods for determining molecules that are linked to ovarian cancer, and the provision of a large collection of such ovarian cancer-linked molecules (as represented for instance by those listed in Table 1), gene expression profiles that provide information on the ovarian cancer-state of a subject are now enabled.

Ovarian cancer-related expression profiles comprise the distinct and identifiable pattern of expression (or level) of sets of ovarian cancer-related genes, for instance a pattern of high and low expression of a defined set of genes, or molecules that can be correlated to such genes, such as mRNA levels or protein levels or activities. Useful sets of molecules for constructing nucleic acid expression profiles include at least one that is represented by the following genes and ESTs: BCKDHB (SEQ ID NO: 16-17), ZNF33A (SEQ ID NO: 20-22), EST 192198 (SEQ ID NO: 25), EST 128738 (SEQ ID NO: 26-27), EST 429211 (28-29), FLJ22174 (SEQ ID NO: 30-31). EST 41556 (SEQ ID NO: 32-33), EST 296488 (SEQ ID NO: 34-35), EST 120124 (SEQ ID NO: 36-37), EST 132142 (SEQ ID NO: 38-39), EST 50635 (SEQ ID NO: 40), POR (SEQ ID NO: 41-43), EST 73702 (SEQ ID NO: 46-47), EST 2218314 (SEQ ID NO: 48), EST 2261113 (SEQ ID NO: 49), IFITM1 (SEQ ID NO: 50-54), IFITM2 (SEQ ID NO: 55-59), KIAA0203 (SEQ ID NO: 61-62). GIP3 (SEQ ID NO: 68-69). BST2 (SEQ ID NO: 70-72), EST 1384797 (SEQ ID NO: 196), TLR3 (SEQ ID NO: 199-201), SPON1 (SEQ ID NO: 160-161), HSRNASEB (SEQ ID NO: 162-163), EST 294506 (SEQ ID NO: 146-148), SORL1 (SEQ ID NO: 149-151), SIAT1 (SEQ ID NO: 73), PL1 (SEQ ID NO: 77), EST 108422 (SEQ ID NO: 78-79), CEBPG (SEQ ID NO: 80), HLA-DPA (SEQ ID NO: 97-99), H2AFL (SEQ ID NO: 107-109). IGKC (SEQ ID NO: 112-116), SCYB10 (SEQ ID NO: 120-121), RGS1 (SEQ ID NO: 122-126), LSR68 (SEQ ID NO: 168), SGK (SEQ ID NO: 176-178), and ZFP36 (SEQ ID NO: 167-173). These genes and ESTs, which have not previously been correlated with cancer, present potentially useful novel markers for cancer, and in particular, ovarian cancer.

A second example set of molecules that could be used in a profile would include at least one that is represented by (or correlated to) the genes and ESTs represented by the SEQ ID NOs in Table 9. These nucleic acids, which are disclosed herein to be differentially expressed in ovarian cancer (see FIG. 2), are suitable for markers to diagnose, prognose, and monitor ovarian cancer in a subject. In addition, these genes and ESTs are potentially useful as markers for classifying tumors into types, for instance into BRCA1-type or BRCA2-type tumors, using the methods disclosed herein.

A third example set of molecules that could be used in a profile would include at least one that is represented by (or correlated to) the genes and ESTs represented by SEQ ID NOs: 417, 284, 285, 281, 283, 278, 273, 282, 274. These represent markers disclosed herein that were found to be differentially expressed between BRCA1-Linked and sporadic tumors in a comparison to reference Immortalized Ovarian Epithelial Cells (IOSE). These markers are useful for classifying tumors into BRCA1-linked and sporadic types, and present potential targets for treatment of ovarian cancer.

A fourth example set of molecules that could be used in a profile would include at least one that is represented by (or correlated to) the genes and ESTs represented by SEQ ID NOs: 279-280, which, as disclosed herein, are markers that were found to be differentially expressed between BRCA2-Linked and sporadic tumors in a comparison to reference Immortalized Ovarian Epithelial Cells. These markers are useful for classifying tumors into BRCA2-linked and sporadic types, and present potential targets for treatment of ovarian cancer.

A fifth example set of molecules that could be used in a profile would include at least one that is represented by (or correlated to) the genes and ESTs represented by SEQ ID NOs: 281, 282 and 274, which, as disclosed herein, are markers that were found to be differentially expressed between combined BRCA-Linked and sporadic tumors in a comparison to reference Immortalized Ovarian Epithelial Cells. These markers are useful for classifying tumors into BRCA-linked and sporadic types, and present potential targets for treatment of ovarian cancer.

A sixth example set of molecules that could be used in a profile would include at least one that is represented by (or correlated to) the genes and ESTs represented by the SEQ ID NOs set forth in Table 10, which, as disclosed herein, are markers that can be used to segregate BRCA1-linked from BRCA2-linked tumor types using compound covariate prediction analysis. These markers are useful for classifying tumors into one of two types of tumors, which provides information helpful to a clinician in choosing a course of treatment for the patient based on the type of tumor into which the sample is classified.

A seventh example set of molecules that could be used in a profile would include at least one that is represented by (or correlated to) the genes and ESTs represented by SEQ ID NO: 16-201, 565-567, and 803-804. These genes and ESTs were found, as disclosed herein, to be differentially expressed in a comparison of BRCA1-linked and BRCA2-linked to sporadic tumors. Hence, these genes and ESTs present potentially useful markers for classifying tumors into types, using the methods disclosed herein. Furthermore, they represent potential targets for pharmaceutical treatment of tumors of each respective tumor type.

A eighth example set of molecules that could be used in a profile would include at least one that is represented by (or correlated to) the genes and ESTs represented by SEQ ID NOs: 124-126, 319, 429-430, 504-523, 533-535, 544, and 548-799. As disclosed herein, these nucleic acids were found to be overexpressed in a comparison of BRCA1-linked, BRCA2-linked and sporadic tumor samples. Hence, these genes and ESTs present potentially useful markers for classifying tumors into types, using the methods disclosed herein. Furthermore, they represent potential targets for pharmaceutical treatment of tumors of each respective tumor type.

A ninth example set of molecules that could be used in a profile would include at least one that is represented by (or correlated to) the genes and ESTs represented by SEQ ID NOs: 202-339. As disclosed herein, these nucleic acids were found to be overexpressed in ovarian cancer in a comparison of ovarian epithelial cancer to normal postmenopausal ovarian tissue. Hence, these genes and ESTs present potentially useful markers diagnosis, prognosis, and monitoring of ovarian cancer. In addition, they represent potential targets for pharmaceutical treatment of ovarian tumors.

A tenth example set of molecules that could be used in a profile would include at least one that is represented by (or correlated to) the genes and ESTs represented by SEQ ID NOs:97 and 340-448. As disclosed herein, these nucleic acids were found to be underexpressed in ovarian cancer in a comparison of ovarian epithelial cancer to normal postmenopausal ovarian tissue. Hence, these genes and ESTs present potentially useful markers diagnosis, prognosis, and monitoring of ovarian cancer. In addition, they represent potential targets for pharmaceutical treatment of ovarian tumors.

In other examples of ovarian cancer-related gene expression profiles, such profiles may be further broken down by the manner of molecules included in the profile. Thus, certain examples of profiles may include a specific class of ovarian cancer markers, such as those molecules involved in cell cycle control.

Particular profiles may be specific for a particular stage of normal tissue (e.g., ovarian tissue) growth or disease progression (e.g., progression of ovarian cancer). Thus, gene expression profiles can be established for a pre-ovarian cancer tissue (i.e., normal ovarian tissue), and a primary ovarian cancer tissue. Each of these profiles includes information on the expression level of at least one, but usually two or more, genes that are linked to ovarian cancer (e.g., ovarian cancer-related genes). Such information can include relative as well as absolute expression levels of specific genes. Likewise, the value measured may be the relative or absolute level of protein expression, which can be correlated with a “gene expression level.” Results from the gene expression profiles of an individual subject are often viewed in the context of a test sample compared to a baseline or control sample fingerprint.

The levels of molecules that make up a gene expression profile can be measured in any of various known ways, which may be specific for the type of molecule being measured. Thus, nucleic acid levels (such as direct gene expression levels, such as the level of mRNA expression) can be measured using specific nucleic acid hybridization reactions. Protein levels may be measured using standard protein assays, using immunologic-based assays (such as ELISAs and related techniques), or using activity assays, for instance. Examples for measuring nucleic acid and protein levels are provided herein; other methods are well known to those of ordinary skill in the art.

Examples of ovarian cancer-related gene expression profiles can be in array format, such as a nucleotide (e.g., polynucleotide) or protein array or microarray. The use of arrays to determine the presence and/or level of a collection of biological macromolecules is now well known (see, for example, methods described in published PCT application number WO9948916, describing hypoxia-related gene expression arrays). In array-based measurement methods, an array may be contacted with polynucleotides (in the case of a nucleic acid-based array) or polypeptides (in the case of a protein-based array) from a sample from a subject. The amount and/or position of binding of the subject's polynucleotides or polypeptides then can be determined, for instance to produce a gene expression profile for that subject. Such gene expression profile can be compared to another gene expression profile, for instance a control gene expression profile from a subject having a known gynecological or ovary-related condition. Optionally, the subject's gene expression profile can be correlated with one or more appropriate treatments, which may be correlated with a control (or set of control) expression profiles for stages of ovarian cancer, for instance.

This disclosure provides the identification of ovarian cancer-related molecules that exhibit alterations in expression during development of ovarian cancer, and expression fingerprints (profiles) specific for ovarian cancers. It further provides methods of using these identified nucleic acid molecules, and proteins encoded thereby, and expression fingerprints or profiles, for instance to predict and/or diagnose ovarian cancer, and to elect treatments for instance based on likely response. These identified ovarian cancer-related molecules also can serve as therapeutic targets, and can be used in methods for identifying, developing and testing therapeutic compounds. It will be apparent that the precise details of the methods described may be varied or modified without departing from the spirit of the described invention. We claim all such modifications and variations that fall within the scope and spirit of the claims below.

### Addendum

TABLE 1
Markers that were Differentially Expressed in a cDNA Microarray
Expression Profile of Sixty-One Ovarian Cancer Tumors

| | | | | Gen Bank | |
|---|---|---|---|---|---|
| SEQ ID | | IMAGE | UniGene | Accession | |
| NO. | Gene | ID No. | No. | No. | Gene Description |
| 16-17 | BCKDHB | 770835 | | AA427739; | Branched chain keto acid dehydrogenase |
| | | | | AA434304 | E1, beta polypeptide (maple syrup urine |
| | | | | | disease) |
| 18-19 | SERPINF2 | 82195 | | T68859; | serine (or cysteine) |
| | | | | T68934 | proteinase inhibitor, clade F (alpha-2 |
| | | | | | antiplasmin, pigment epithelium derived |
| | | | | | factor), mem |
| 20-22 | ZNF33A | 346902 | | D31763; | KIAA0065 |
| | | | | W78164; | |
| | | | | W79207 | |
| 23-24 | ZNF33A | 246543 | | N57658; | Zinc finger protein 33a (KOX 31) |
| | | | | N77515 | |
| 25 | EST | 192198 | | H41144 | Unknown |
| 26-27 | EST | 128738 | | R16726; | Homo sapiens cDNA: FLJ23371 fis, |
| | | | | R09980 | clone HEP16068, highly similar to |
| | | | | | HSTFIISH Homo sapiens mRNA for trar |
| 28-29 | EST | 429211 | | AA007283; | ESTs |
| | | | | AA007282 | |
| 30-31 | FLJ22174 | 295939 | | N67034; | hypothetical protein FLJ22174 |
| | | | | W04283 | |
| 32-33 | EST | 415562 | | W80701; | Unknown |
| | | | | W78802 | |
| 34-35 | EST | 296488 | | N70208; | Unknown |
| | | | | W01059 | |
| 36-37 | EST | 120124 | | T95064; | ESTs |
| | | | | T95160 | |
| 38-39 | EST | 132142 | | R26164; | Homo sapiens cDNA: FLJ21587 fis, |
| | | | | R23610 | clone COL06946 |
| 40 | EST | 50635 | | H17921 | ESTs |
| 41-43 | POR | 234180 | | S90469; | Cytochrome P450 reductase |
| | | | | H70626; | |
| | | | | H66247 | |
| 44-45 | CLU | 725877 | | AA292226; | Clusterin (complement lysis inhibitor, |
| | | | | AA292410 | SP-40, 40, sulfated glycoprotein 2, |
| | | | | | testosterone-repressed |
| | | | | | prostate messenger |
| 46-47 | EST | 73702 | | T54544; | Unknown |
| | | | | T54585 | |
| 48 | EST | 2218314 | | AI744768 | Unknown |
| 49 | EST | 2261113 | | AI609063 | EST |
| 50-51 | IFITM1 | 755599 | | AA419251; | Interferon induced transmembrane protein |
| | | | | AA419286 | 1 (9-27) |
| 52-54 | IFITM1 | 509641 | | J04164; | Interferon-inducible protein 9-27 = |
| | | | | AA058323; | interferon-induced 17 kDa membrane |
| | | | | AA058453 | protein |
| 55-57 | IFITM2 | 624655 | | X57351; | Interferon-induced protein 1-8D |
| | | | | AA187365; | |
| | | | | AA187099 | |
| 58-59 | IFITM2 | 376520 | | AA041402; | Interferon-induced protein 1-8D |
| | | | | AA041501 | |
| 60 | HE4 | 786675 | | AA451904 | Epididymis-specific, whey-acidic protein |
| | | | | | type, four-disulfide core; putative ovarian |
| | | | | | carcinoma marker |
| 61-62 | KIAA0203 | 61008 | | T40715; | K1AA0203 gene product |
| | | | | T39659 | |
| 63-64 | IL8RB | 882183 | | M73969; | CXCR2 = IL-8 Receptor beta |
| | | | | AA480683 | |
| 65-67 | VDUP1 | 297954 | | S73591; | Brain-expressed HHCPA78 homolog = Induced in |
| | | | | N68956; | HL60 cells treated with vitamin D or |
| | | | | W00656 | cycloheximide |
| 68-69 | G1P3 | 782513 | | AA448478; | Interferon, alpha-inducible protein |
| | | | | AA432030 | (clone IFI-6-16) |
| 70-72 | BST2 | 811024 | | D28137; | Bone marrow stromal cell antigen 2 |
| | | | | AA485371; | |
| | | | | AA485528 | |
| 73 | SIAT1 | 897906 | | AA598652 | Sialyltransferase 1 (beta-galactoside |
| | | | | | alpha-2,6-sialyltransferase) |
| 74-76 | DDR1 | 182288 | | U48705; | Receptor protein-tyrosine kinase |
| | | | | H41900; | EDDR1 |
| | | | | H41939 | |
| 77 | PL1 | 433573 | | AA701655 | Human endogenous retrovirus envelope |
| | | | | | region mRNA (PL1) |
| 78-79 | EST | 108422 | | T77847; | Homo sapiens, clone MGC: 12275, mRNA, |
| | | | | T77926 | complete cds |
| 80 | CEBPG | 455121 | | AA676804 | CCAAT/enhancer binding protein |
| | | | | | (C/EBP), gamma |
| 81-82 | MUC1 | 840687 | | AA488073; | Mucin 1, transmembrane |
| | | | | AA486365 | |
| 83-84 | CP | 223350 | | H86554; | ceruloplasmin (ferroxidase) |
| | | | | H86642 | |
| 85-86 | HLA-DRB5 | 811139 | | AA485739; | Major histocompatibility complex, |
| | | | | AA486460 | class II, DR beta 5 |
| 87-88 | HLA-DRB1 | 417711 | | W88967; | Major histocompatibility |
| | | | | W88546 | complex, class II, DR beta 1 |
| 89-91 | CD74 | 725751 | | X00497; | Invariant chain = la-associated invariant |
| | | | | AA399225; | gamma-chain |
| | | | | AA292218 | |
| 92-93 | CD74 | 840681 | | AA488071; | Invariant chain = la-associated invariant |
| | | | | AA486363 | gamma-chain |
| 94-96 | HLA-DRA | 117411 | | K01171; | MHC Class II = DR alpha |
| | | | | T89719; | |
| | | | | T89816 | |
| 97-99 | HLA-DPA | 207715 | | X00457; | MHC Class II = DP alpha |
| | | | | H62294; | |
| | | | | H62293 | |
| 100 | HLA-DRB1 | 855547 | | AA664195 | Major histocompatibility complex, |
| | | | | | class II, DR beta 1 |
| 101-103 | HLA-DRB1 | 470953 | | M20430; | MHC Class II = DR beta |
| | | | | AA032179; | |
| | | | | AA033653 | |
| 104-106 | TNFSF10 | 203132 | | U57059; | TRAIL = Apo-2 ligand |
| | | | | H54629; | |
| | | | | H54628 | |
| 107-109 | H2AFL | 429091 | | U90551; | Histone-2A-like protein (H2A/I) |
| | | | | AA007585; | |
| | | | | AA007574 | |
| 110-111 | IG | 66560 | | T67053; | Immunoglobulin lambda locus |
| | lambda | | | T67054 | |
| 112-114 | IGKC | 159142 | | M63438; | Immunoglobulin kappa light chain |
| | | | | R76324; | |
| | | | | R76323 | |
| 115-116 | IGKC | 840451 | | AA485725; | Immunoglobulin kappa light chain |
| | | | | AA485862 | |
| 117-119 | RAD23A | 293925 | | AF004230; | MIR-7 = monocyte/macrophage lg-related receptor |
| | | | | N63943; | AND RAD23 = UV excision repair protein |
| | | | | N98412 | (Double hit) |
| 120-121 | SCYB10 | 967284 | | X02530; | IP-10 |
| | | | | AA527139 | |
| 122-123 | RGS1 | 361323 | | AA017544; | regulator of G-protein signaling 1 |
| | | | | AA017417 | |
| 124-126 | RGS1 | 686248 | | S59049; | BL34 = RGS1 = regulator of G-protein |
| | | | | AA262268; | signaling which inhibits SDF-1 |
| | | | | AA262879 | directed B cell migration |
| 127-129 | GAS1 | 365826 | | L13698; | Growth arrest-specific 1 |
| | | | | AA025819; | |
| | | | | AA025884 | |
| 130-132 | BTG2 | 358214 | | U72649; | BTG2 = p53 dependent inducible anti- |
| | | | | W95415; | proliferative gene homologous to |
| | | | | W95512 | Pc3/Tis21 immediate early genes |
| 133-135 | FOS | 755279 | | V01512; | c-fos |
| | | | | AA496353; | |
| | | | | AA496403 | |
| 136 | LSR68 | 1862044 | | AI053597 | Lipopolysaccharide specific response-68 |
| | | | | | protein |
| 137-138 | JUNB | 309864 | | N94468; | Jun B proto-oncogene |
| | | | | W23847 | |
| 139-140 | JUNB | 122428 | | T99236; | Jun B proto-oncogene |
| | | | | T99280 | |
| 141-143 | COL3A1 | 122159 | | X14420; | Collagen Type III Alpha 1 |
| | | | | T98612; | |
| | | | | T98611 | |
| 144-145 | LUM | 813823 | | AA447781; | lumican |
| | | | | AA453712 | |
| 146-148 | EST | 294506 | | U90916; | clone 23815 mRNA |
| | | | | N71007; | |
| | | | | W01902 | |
| 149-151 | SORL1 | 279388 | | Y08110; | Mosaic protein LR11 = hybrid receptor |
| | | | | N48698; | gp250 precursor |
| | | | | N45548 | |
| 152-153 | RNASE6PL | 712341 | | AA405000; | Ribonuclease 6 precursor |
| | | | | AA281840 | |
| 154-156 | HLA-B | 769753 | | M28205; | Human Leukocyte Antigen B |
| | | | | AA429012; | |
| | | | | AA429162 | |
| 157-159 | HLA-C | 810142 | | M11886; | Human Leukocyte Antigen C |
| | | | | AA464246; | |
| | | | | AA464354 | |
| 160-161 | SPON1 | 46173 | | H09099; | Spondin 1, (f-spondin) extracellular |
| | | | | H09449 | matrix protein |
| 162-163 | HSRNASEB | 814526 | | AA459363; | RNA-binding region (RNP1, RRM) |
| | | | | AA459588 | containing 1 |
| 164-166 | ABCB1 | 813256 | | M14758; | MDR1 = Multidrug resistance protein |
| | | | | AA455911; | 1 = P-glycoprotein |
| | | | | AA456377 | |
| 167-168 | ZFP36 | 23804 | | R38383; | Zinc finger protein homologous to Zfp-36 |
| | | | | T77499 | in mouse |
| 169-171 | ZFP36 | 135880 | | M63625; | TTP = tristetraproline = GOS24 = zinc finger |
| | | | | R33813; | transcriptional regulator |
| | | | | R33812 | |
| 172-173 | ZFP36 | 727266 | | AA411987; | TTP = tristetraproline = GOS24 = zinc finger |
| | | | | AA402178 | transcriptional regulator |
| 174-175 | HGF | 41650 | | R52798; | hepatocyte growth factor (hepapoietin A; |
| | | | | R52797 | scatter factor) |
| 176-178 | SGK | 840776 | | AJ000512; | sgk = putative serine/threonine |
| | | | | AA486082; | protein kinase transcriptionally |
| | | | | AA486140 | modified during anisotonic and isotonic |
| | | | | | alteration |
| 179-180 | PPP1R7 | 814508 | | AA459351; | Protein phosphatase 1, regulatory subunit 7 |
| | | | | AA459572 | |
| 181-182 | CD24 | 204335 | | H59916; | CD24 antigen (small cell lung carcinoma |
| | | | | H59915 | cluster 4 antigen) |
| 183-184 | TPD52 | 814306 | | AA459100; | Tumor protein D52 |
| | | | | AA459318 | |
| 185-187 | CXCR4 | 79629 | | X71635; | CXC chemokine receptor 4 = |
| | | | | T62636; | fusin = neuropeptide Y receptor = L3 |
| | | | | T62491 | |
| 188-189 | JUND | 767784 | | AA418670; | Jun D proto-oncogene |
| | | | | AA418676 | |
| 190-192 | BRF2 | 485770 | | U07802; | Tis 11d = ERF-2 = growth factor early |
| | | | | AA039882; | response gene |
| | | | | AA039967 | |
| 193-195 | A2M | 377647 | | M11313; | Alpha-2-macroglobulin |
| | | | | AA055995; | |
| | | | | AA055907 | |
| 196 | EST | 1384797 | | AA856938 | Homo sapiens mRNA; cDNA DKFZp434O0227 |
| | | | | | (from clone DKFZp434O0227) |
| 197-198 | CD24 | 196519 | | S75311; | CD24 |
| | | | | R91610 | |
| 199-201 | TLR3 | 144675 | | U88879; | TLR3 = Toll-like receptor 3 |
| | | | | R76099; | |
| | | | | R76150 | |
| 202 | ITM2A | 878596 | Hs.17109 | AA775257 | integral membrane protein 2A |
| 203-204 | GATM | 42558 | Hs.75335 | R61229; | glycine amidinotransferase (L- |
| | | | | R61228 | arginine:glycine amidinotransferase) |
| 205-207 | RNASE4 | 81417 | Hs.283749 | D37931; | ribonuclease L (2′,5′-oligoisoadenylate |
| | | | | T60163; | synthetase-dependent) |
| | | | | T60223 | |
| 208-210 | LAMA2 | 471642 | Hs.75279 | Z26653; | laminin alpha 2 (merosin, congenital |
| | | | | AA034939; | muscular dystrophy) |
| | | | | AA034938 | |
| 211 | PBX3 | 448386 | Hs.294101 | AA778198 | pre-B-cell leukemia transcription factor 3 |
| 212 | PLA2G6 | 1472538 | Hs.120360 | AA872271 | phospholipase A2, group VI (cytosolic, |
| | | | | | calcium-independent) |
| 213 | SMARCA2 | 814636 | Hs.198296 | AA481026 | SWI/SNF related, matrix associated, |
| | | | | | actin dependent regulator of chromatin, |
| | | | | | subfamily a, member 2 |
| 214-215 | CUGBP2 | 488956 | Hs.211610 | AA047257; | CUG triplet repeat, RNA-binding |
| | | | | AA057142 | protein 2 |
| 216-218 | TGFBR3 | 209655 | Hs.79059 | L07594; | TGF beta receptor type III |
| | | | | H62473; | |
| | | | | H61499 | |
| 219 | STAR | 859858 | Hs.3132 | AA679454 | steroidogenic acute |
| | | | | | regulatory protein |
| 220 | GNG11 | 1636447 | Hs.83381 | AA999901 | guanine nuclcotide binding protein 11 |
| 221 | CITED2 | 491565 | Hs.82071 | AA115076 | Cbp/p300-interacting transactivator, with |
| | | | | | Glu/Asp-rich carboxy-terminal domain, 2 |
| 222 | CTNNAL1 | 744647 | Hs.58488 | AA621315 | catenin (cadherin-associated protein), |
| | | | | | alpha-like 1 |
| 223 | ABCA8 | 743773 | Hs.38095 | AA634308 | ATP-binding cassette, sub-family A (ABC1), |
| | | | | | member 8 |
| 224-226 | KLF4 | 188232 | Hs.7934 | AF105036; | GKLF = EZF = KLF4 = gut-enriched Kruppel- |
| | | | | H45668; | like zinc finger protein = expressed in |
| | | | | H45711 | vascular endothelial cells |
| 227-228 | ITPR1 | 471725 | Hs.198443 | AA035450; | inositol 1,4,5-triphosphate receptor, |
| | | | | AA035477 | type 1 |
| 229-230 | MAF | 487793 | Hs.30250 | AA043501; | v-maf musculoaponeurotic fibrosarcoma |
| | | | | AA044658 | (avian) oncogene homolog |
| 231-232 | FOXC1 | 768370 | Hs.284186 | AA495790; | forkhead box C1 |
| | | | | AA495846 | |
| 233 | TCF21 | 461351 | Hs.78061 | AA699782 | transcription factor 21 |
| 234-236 | CCNI | 248295 | Hs.79933 | D50310; | Cyclin I |
| | | | | N58511; | |
| | | | | N78101 | |
| 237-238 | DCN | 209367 | Hs.76152 | H64138; | decorin |
| | | | | H64086 | |
| 239-240 | CBF2 | 789049 | Hs.184760 | AA452909; | CCAAT-box-binding transcription factor |
| | | | | AA453077 | |
| 241-242 | EST | 68049 | Hs.180324 | T52830; | Homo sapiens, clone IMAGE: 4183312, |
| | | | | T52829 | mRNA, partial cds |
| 122 | RNASE4 | 81417 | Hs.283749 | | ribonuclease L (2′,5′-oligoisoadenylate |
| | | | | | synthetase-dependent) |
| 243 | SLC4A1AP | 2094012 | Hs.306000 | AI424433 | solute carrier family 4 (anion exchanger), |
| | | | | | member 1, adapter protein |
| 244-245 | GSTM5 | 377731 | Hs.75652 | AA056232; | glutathione S-transferase M5 |
| | | | | AA056231 | |
| 246 | C4BPB | 460470 | Hs.99886 | AA677687 | complement component 4-binding |
| | | | | | protein, beta |
| 247-248 | HS3ST1 | 73609 | Hs.40968 | T55714; | heparan sulfate (glucosamine) 3-O- |
| | | | | T55756 | sulfotransferase 1 |
| 249 | CDKN1C | 2413955 | Hs.106070 | AI828088 | cyclin-dependent kinase inhibitor 1C |
| | | | | | (p57, Kip2) |
| 250 | HNRPDL | 897823 | Hs.170311 | AA598578 | heterogeneous nuclear ribonucleoprotein D-like |
| 251 | CIRBP | 1558799 | Hs.119475 | AA977242 | cold inducible RNA-binding protein |
| 252 | RGS2 | 2321596 | Hs.78944 | A1675670 | regulator of G-protein signalling 2, 24 kD |
| 253-254 | TCEAL1 | 786607 | Hs.95243 | AA478480; | transcription elongation factor A |
| | | | | AA451969 | (SII)-like 1 |
| 255-256 | CAV1 | 377461 | Hs.323469 | AA055835; | caveolin 1, caveolae protein, 22 kD |
| | | | | AA055368 | |
| 257 | ALDH1A1 | 855624 | Hs.76392 | AA664101 | aldehyde dehydrogenase 1 family, member A1 |
| 258-259 | RBPMS | 343443 | Hs.80248 | W67200; | RNA-binding protein gene with multiple |
| | | | | W67323 | splicing |
| 260-261 | ADAMTS1 | 62263 | Hs.8230 | T41173; | a disintegrin-like and metalloprotease |
| | | | | T40309 | (reprolysin type) with thrombospondin |
| | | | | | type 1 motif, 1 |
| 262-263 | DSCR1L1 | 51408 | Hs.156007 | H19440; | Down syndrome critical region gene |
| | | | | H19439 | 1-like 1 |
| 264 | DLK1 | 436121 | Hs.169228 | AA701996 | delta-like homolog (Drosophila) |
| 265-266 | CDH11 | 491113 | Hs.75929 | AA136983; | cadherin 11, type 2, OB-cadherin |
| | | | | AA137109 | (osteoblast) |
| 138 | SGK | 840776 | Hs.296323 | | sgk = putative serine/threonine |
| | | | | | protein kinase transcriptionally |
| | | | | | modified during anisotonic and isotonic |
| | | | | | alterations of cell volume |
| 267 | HFL1 | 450060 | Hs.278568 | AA703392 | H factor (complement)-like 1 |
| 268-269 | FOG2 | 38347 | Hs.106309 | R49439; | Friend of GATA2 |
| | | | | R35921 | |
| 270 | C7 | 898122 | Hs.78065 | AA598478 | complement component 7 |
| 271-272 | SGCE | 784109 | Hs.110708 | AA432066; | sarcoglycan, epsilon |
| | | | | AA446750 | |
| 273 | NBL1 | 898305 | Hs.76307 | AA598830 | neuroblastoma, suppression of tumorigenicity 1 |
| 274-275 | HBB | 173385 | Hs.155376 | H20968; | hemoglobin, beta |
| | | | | H21011 | |
| 276-278 | CARP | 840683 | Hs.31432 | X83703; | Cytokine inducible nuclear protein |
| | | | | AA488072; | |
| | | | | AA486364 | |
| 279-280 | MITF | 278570 | Hs.166017 | N66177; | microphthalmia-associated transcription factor |
| | | | | N99168 | |
| 281-282 | CDC20 | 898062 | Hs.82906 | U05340; | p55CDC |
| | | | | AA598776 | |
| 283-285 | EPS8 | 148028 | Hs.2132 | U12535; | epidermal growth factor receptor kinase |
| | | | | H13623; | substrate (Eps8) |
| | | | | H13622 | |
| 286-287 | ARHI | 345680 | Hs.194695 | W72033; | ras homolog gene family, member I |
| | | | | W76278 | |
| 288 | B4-2 | 857002 | Hs.75969 | AA669637 | proline-rich protein with nuclear targeting |
| | | | | | signal |
| 289-291 | SELE | 186132 | Hs.89546 | M30640; | ELAM1 = endothelial leukocyte adhesion |
| | | | | H39991; | molecule I |
| | | | | H39560 | |
| 292-293 | PMP22 | 133273 | Hs.103724 | R26732; | peripheral myelin protein 22 |
| | | | | R26960 | |
| 294 | EBAF | 340657 | Hs.25195 | W56771 | endometrial bleeding associated factor (left- |
| | | | | | right determination, factor A; transforming |
| | | | | | growth factor beta superfamily) |
| 295-296 | PRKAR2B | 609663 | Hs.77439 | AA180007; | protein kinase, cAMP-dependent, regulatory, |
| | | | | AA181500 | type II, beta |
| 297 | NFKBIE | 1573311 | Hs.91640 | AA953975 | nuclear factor of kappa light polypeptide gene |
| | | | | | enhancer in B-cells inhibitor, epsilon |
| 298-299 | KIT | 269806 | Hs.81665 | N24824; | v-kit Hardy-Zuckerman 4 feline |
| | | | | N36279 | sarcoma viral oncogene homolog |
| 158 | JUNB | 309864 | Hs.198951 | | jun B proto-oncogene |
| 300 | BCKDK | 1573108 | Hs.20644 | AA970731 | branched chain alpha-ketoacid |
| | | | | | dehydrogenase kinase |
| 301-303 | BTG1 | 298268 | Hs.77054 | X61123; | BTG1 = B-cell translocation gene |
| | | | | N70463; | 1 = anti-proliferative |
| | | | | W03824 | |
| 304-305 | AKAP12 | 784772 | Hs.788 | AA478543; | A kinase (PRKA) anchor protein (gravin) 12 |
| | | | | AA478542 | |
| 306-307 | NR4A2 | 898221 | Hs.82120 | S77154; | NOT = Immediate early response |
| | | | | AA598611 | protein = Nurr1 homologue = Nurr77 |
| | | | | | orphan steroid receptor family member |
| 308-309 | HBB | 126531 | Hs.155376 | R06757; | hemoglobin, beta |
| | | | | R06806 | |
| 310-311 | ARHGAP6 | 768489 | Hs.250830 | AA495981; | Rho GTPase activating protein 6 |
| | | | | AA425035 | |
| 312 | PLS3 | 1568391 | Hs.4114 | AA953747 | plastin 3 (T isoform) |
| 313-314 | FNTA | 300015 | Hs.138381 | N78902; | farnesyltransferase, CAAX box, alpha |
| | | | | W06970 | |
| 315-316 | TNFAIP3 | 770670 | Hs.211600 | AA476272; | tumor necrosis factor, alpha-induced |
| | | | | AA433807 | protein 3 |
| 317-318 | EGR1 | 840944 | Hs.326035 | AA486533; | early growth response 1 |
| | | | | AA486628 | |
| 319 | RNAC | 795213 | Hs.113052 | AA453591 | RNA cyclase homolog |
| 320 | PA26 | 813584 | Hs.14125 | AA447661 | p53 regulated PA26 nuclear protein |
| 321 | C11orf13 | 1573778 | Hs.72925 | AA970526 | chromosome 11 open reading frame 13 |
| 322 | ING1L | 2169465 | Hs.107153 | AI564029 | inhibitor of growth family, member 1-like |
| 323 | RPL9 | 2577249 | Hs.157850 | AW075605 | ribosomal protein L9 |
| 324-325 | ADH5 | 813711 | Hs.78989 | AA453776; | alcohol dehydrogenase 5 (class III), chi |
| | | | | AA453859 | polypeptide |
| 326-327 | FZD7 | 298122 | Hs.173859 | N69049; | frizzled (Drosophila) homolog 7 |
| | | | | W00697 | |
| 328-329 | MATN2 | 366100 | Hs.19368 | AA071473; | matrilin 2 |
| | | | | AA082338 | |
| 330-331 | SLC11A3 | 71863 | Hs.5944 | T52564; | solute carrier family 11 (proton-coupled |
| | | | | T57235 | divalent metal ion transporters), member 3 |
| 332-333 | EST | 767641 | Hs.122460 | AA418293; | ESTs |
| | | | | AA418356 | |
| 334-335 | ERCC5 | 292463 | Hs.48576 | N62586; | excision repair cross-complementing rodent |
| | | | | N80359 | repair deficiency, complementation |
| | | | | | group 5 (xeroderma pigmentosum, |
| | | | | | complementation group G (Cockayne |
| | | | | | syndrome)) |
| 336-337 | MGC2479 | 43771 | Hs.79625 | H05655; | hypothetical protein MGC2479 |
| | | | | H05654 | |
| 338-339 | RPL21 | 810617 | Hs.184108 | AA464743; | ribosomal protein L21 |
| | | | | AA464034 | |
| 182 | CD24 | 204335 | Hs.286124 | | CD24 |
| 340-341 | SLPI | 378813 | Hs.251754 | X04470; | Secretory leukocyte protease inhibitor |
| | | | | AA683520 | |
| 342 | SPP1 | 378461 | Hs.313 | AA775616 | Secreted phosphoprotein 1(osteopontin, |
| | | | | | bone sialoprotein I, early T-lymphocyte |
| | | | | | activation 1) |
| 343-344 | BF | 741977 | Hs.69771 | L15702; | B-factor, properdin |
| | | | | AA401441 | |
| 345-347 | CKS1 | 810899 | Hs.334883 | X54941; | CDC28 protein kinase 1 |
| | | | | AA459292; | |
| | | | | AA459522 | |
| 348-349 | MMP7 | 470393 | Hs.2256 | AA031514; | Matrix metalloproteinase 7 (matrilysin) |
| | | | | AA031513 | |
| 350-351 | PAX8 | 742101 | Hs.73149 | AA405767; | Paired box gene 8 |
| | | | | AA405891 | |
| 352-353 | SPINT2 | 814378 | Hs.31439 | AA458849; | Serine protease inhibitor, Kunitz type, 2 |
| | | | | AA459039 | |
| 354 | ZWINT | 451907 | Hs.42650 | AA706968 | ZW10 interactor |
| 355 | DGKH | 2544675 | Hs.159073 | AW052032 | Diacylglycerol kinase, eta |
| 356 | HMGIY | 782811 | Hs.139800 | AA448261 | HMGIY High-mobility group (nonhistone |
| | | | | | chromosomal) protein isoforms I and Y |
| 357-359 | SDC4 | 504763 | Hs.252189 | X67016; | Syndecan 4 (amphiglycan, ryudocan) |
| | | | | AA148737; | |
| | | | | AA148736 | |
| 360 | CDKN2A | 1161155 | Hs.1174 | AA877595 | Cyclin-dependent kinase inhibitor 2A |
| | | | | | (melanoma, p16, inhibits CDK4) |
| 361-362 | SCNN1A | 810873 | Hs.2794 | AA458982; | Sodium channel, nonvoltage-gated 1 |
| | | | | AA459197 | alpha |
| 363 | LDHA | 43550 | Hs.2795 | H05914 | Lactate dehydrogenase A |
| 364-365 | FOLR1 | 131839 | Hs.73769 | R24530; | Folate receptor 1 |
| | | | | R24635 | |
| 366-367 | TPI1 | 855749 | Hs.83848 | M10036; | Triosephosphate isomerase 1 |
| | | | | AA663983 | |
| 368 | KLK8 | 2514426 | Hs.104570 | AI963941 | Kallikrein 8 (neuropsin/ovasin) |
| 200 | CXCR4 | 79629 | Hs.89414 | | Chemokine (C-X-C motif), receptor 4 |
| | | | | | (fusin) |
| 369-370 | KNSL1 | 825606 | Hs.8878 | AA504625; | Kinesin-like 1 |
| | | | | AA504719 | |
| 371-372 | H2AFO | 488964 | Hs.795 | AA047260; | H2A histone family, member O |
| | | | | AA057146 | |
| 373-374 | HLA-DRA | 153411 | Hs.76807 | R47979; | Major histocompatibility complex, class |
| | | | | R48091 | II, DR alpha |
| 375 | CRIP1 | 1323448 | Hs.17409 | AA873604 | Cysteine-rich protein 1 |
| 376 | PP | 950700 | Hs.184011 | AA608572 | pyrophosphatase (inorganic) |
| 377-378 | EST | 666391 | | AA232895; | |
| | | | | AA232894 | |
| 379-381 | SLC2A1 | 207358 | Hs.169902 | K03195; | Solute carrier family 2 (facilitated glucose |
| | | | | H58873; | transporter), member 1 |
| | | | | H58872 | |
| 382 | EST | 897770 | | AA598508 | |
| 383-385 | HDGF | 813673 | Hs.89525 | D16431; | Hepatoma-derived growth factor (high- |
| | | | | AA453749; | mobility group protein 1-like) |
| | | | | AA453831 | |
| 386 | ASS | 882522 | Hs.160786 | AA676466 | Argininosuccinate synthetase |
| 387-388 | CLDN4 | 770388 | Hs.5372 | AA430665; | Claudin 4 |
| | | | | AA427468 | |
| 389 | PRAME | 897956 | Hs.30743 | AA598817 | preferentially expressed antigen in |
| | | | | | melanoma |
| 390-391 | PTPRF | 897788 | Hs.75216 | Y00815; | Protein tyrosine phosphatase, receptor |
| | | | | AA598513 | type, F |
| 392-393 | EYA2 | 741139 | Hs.29279 | AA402754; | Eyes absent (Drosophila) homolog 2 |
| | | | | AA402207 | |
| 394-396 | MYCL1 | 138917 | Hs.92137 | M19720; | v-myc myclocytomatosis viral oncogene |
| | | | | R62813; | homolog 1 |
| | | | | R62862 | |
| 397-399 | STAT1 | 840691 | Hs.21486 | M97935; | Signal transducer and activator of |
| | | | | AA488075; | transcription 1 |
| | | | | AA486367 | |
| 400-401 | MTCH2 | 564492 | Hs.279609 | AA121668; | mitochondrial carrier homolog 2 |
| | | | | AA121740 | |
| 402 | HTR3A | 435597 | Hs.2142 | AA703169 | 5-hydroxytryptamine (serotonin) receptor 3A |
| 403-404 | CCNE1 | 68950 | Hs.9700 | T54121; | Cyclin E1 |
| | | | | T54213 | |
| 405 | CDH6 | 739155 | Hs.32963 | AA421819 | Cadherin 6, type 2, K-cadherin |
| 406-408 | PRKAG1 | 531028 | Hs.3136 | U42412; | Protein kinase, AMP-activated, gamma 1 |
| | | | | AA070495; | non-catalytic subunit |
| | | | | AA070381 | |
| 409 | DEFB1 | 2403485 | Hs.32949 | AI769855 | Defensin, beta 1 |
| 410-411 | ARPC1B | 626502 | Hs.11538 | AA188179; | Actin related protein 2/3 complex, subunit |
| | | | | AA188155 | 1B (41 kD) |
| 412-414 | PRKCI | 71622 | Hs.1904 | L33881; | Protein kinase C, iota |
| | | | | T57875; | |
| | | | | T57957 | |
| 415 | GAPD | 1610448 | Hs.169476 | AA991856 | glyceraldehyde-3-phosphate dehydrogenase |
| 416-417 | C2 | 85497 | Hs.2253 | T71879; | Complement Component C2 |
| | | | | T71878 | |
| 418-419 | H2AFY | 843075 | Hs.75258 | AA488627; | H2A histone family, member Y |
| | | | | AA486003 | |
| 420-421 | TM4SF1 | 840567 | Hs.3337 | AA487893; | Transmembrane 4 superfamily member 1 |
| | | | | AA488005 | |
| 422-423 | GAPD | 50117 | Hs.169476 | H16958; | glyceraldehyde-3-phosphate dehydrogenase |
| | | | | H16957 | |
| 424-426 | IFITM3 | 809910 | Hs.182241 | X57352; | interferon induced transmembrane protein |
| | | | | AA464417; | 3 (1-8U) |
| | | | | AA464416 | |
| 427-428 | GLDC | 248261 | Hs.27 | N58494; | Glycine dehydrogenase (decarboxylating; |
| | | | | N78083 | glycine decarboxylase, glycine cleavage |
| | | | | | system protein P) |
| 429-430 | CALU | 144881 | Hs.7753 | R78586; | Calumenin |
| | | | | R78585 | |
| 431-432 | HBA2 | 208764 | Hs.272572 | H63096; | Hemoglobin, alpha 2 |
| | | | | H63182 | |
| 433 | S100A11 | 810612 | Hs.256290 | AA464731 | S100 calcium-binding protein A11 (calgizzarin) |
| 434-436 | LDHA | 897567 | Hs.2795 | X02152; | Lactate dehydrogenase A |
| | | | | AA497029; | |
| | | | | AA489611 | |
| 437 | UBE2C | 769921 | Hs.93002 | AA430504 | Ubiquitin-conjugating enzyme E2C |
| 438-440 | E2F3 | 304908 | Hs.1189 | D38550; | E2F transcription factor 3 |
| | | | | N92519; | |
| | | | | W38841 | |
| 441-442 | CDH1 | 251019 | Hs.194657 | Z13009; | Cadherin 1, type 1, E-cadherin (epithelial) |
| | | | | H97778 | |
| 443-444 | PSME2 | 210405 | Hs.179774 | H65395; | Proteasome (prosome, macropain) activator |
| | | | | H65394 | subunit 2 (PA28 beta) |
| 445-447 | BMP7 | 344430 | Hs.170195 | X51801; | bone morphogenetic protein 7 (osteogenic |
| | | | | W73473; | protein 1) |
| | | | | W73527 | |
| 448 | TOP2A | 825470 | | AA504348 | topoisomerase (DNA) II alpha (170 kD) |
| 449-451 | IL8 | 328692 | Hs.624 | M17017; | interleukin 8 |
| | | | | W45324; | |
| | | | | W40283 | |
| 452-453 | GRO1 | 324437 | Hs.789 | X54489; | GRO1 oncogene (melanoma growth stimulating |
| | | | | W46900 | activity, alpha) |
| 454-456 | ALDH1A3 | 272686 | Hs.75746 | U07919; | aldehyde dehydrogenase 1 family, member A3; |
| | | | | N32289; | Aldehyde dehydrogenase 6 |
| | | | | N44575 | |
| 457-459 | MMP1 | 624924 | Hs.83169 | X54925; | matrix metalloproteinase 1 (interstitial |
| | | | | AA181875; | collagenase) |
| | | | | AA186634 | |
| 460-461 | OSF-2 | 897910 | Hs.136348 | D13666; | osteoblast specific factor 2 (fasciclin I- |
| | | | | AA598653 | like) |
| 462-464 | CDC25B | 48398 | Hs.153752 | S78187; | cell division cycle 25B; M-phase inducer |
| | | | | H14343; | phosphatase 2 |
| | | | | H14392 | |
| 465-467 | FLNA | 487418 | Hs.195464 | X53416; | filamin A, alpha (actin-binding protein- |
| | | | | AA046721; | 280) |
| | | | | AA046606 | |
| 468-469 | TFP12 | 726086 | Hs.295944 | AA399473; | tissue factor pathway inhibitor 2 |
| | | | | AA293402 | |
| 470-472 | FGF2 | 23073 | Hs.284244 | M27968; | fibroblast growth factor 2 (basic) |
| | | | | R38539; | |
| | | | | T75110 | |
| 473-475 | CD44 | 713145 | Hs.169610 | X56794; | CD44 antigen; extracellular matrix |
| | | | | AA283090; | receptor-III = Hyaluronate |
| | | | | AA282906 | receptor |
| 476-477 | DYT1 | 69046 | Hs.19261 | T54320; | dystonia 1, torsion (autosomal dominant; |
| | | | | T53726 | torsin A) |
| 478 | UCHL1 | 878833 | Hs.76118 | AA670438 | ubiquitin carboxyl-terminal esterase L1 |
| | | | | | (ubiquitin thiolesterase) |
| 479-480 | PLAU | 1696513 | Hs.77274 | D00244; | plasminogen activator, urokinase |
| | | | | AI088434 | |
| 256 | LDHA | 43550 | Hs.2795 | | lactate dehydrogenase A |
| 481-483 | PTGS2 | 147050 | Hs.196384 | U04636; | cyclooxygenase-2; prostaglandin |
| | | | | R80217; | endoperoxide synthase-2 |
| | | | | R80322 | |
| 484-486 | PRNP | 682013 | Hs.74621 | M13899; | prion protein |
| | | | | AA256322; | |
| | | | | AA256449 | |
| 487-488 | MT1X | 297392 | Hs.278462 | N80129; | metallothionein 1L, metallothionein 1X |
| | | | | W03653 | |
| 489-490 | UGB | 81336 | Hs.2240 | T63761; | uteroglobin |
| | | | | T63800 | |
| 491-493 | PBEF | 594539 | Hs.239138 | U02020; | pre-B-cell colony-enhancing factor |
| | | | | AA169813; | |
| | | | | AA171651 | |
| 494-496 | TXNRD1 | 789376 | Hs.13046 | X91247; | thioredoxin reductase 1; GRIM-12 |
| | | | | AA464849; | |
| | | | | AA453335 | |
| 497-499 | NT5 | 21655 | Hs.153952 | X55740; | 5′ nucleotidase (CD73) |
| | | | | T65120; | |
| | | | | T65189 | |
| 500-502 | MT2A | 590150 | Hs.118786 | J00271; | metallothionein 2A |
| | | | | AA156031; | |
| | | | | AA156201 | |
| 503 | ZNF220 | 949928 | Hs.82210 | AA599173 | zinc finger protein 220 |
| 504-506 | AKT1 | 810331 | Hs.71816 | U97276; | BPGF-1 = bone-derived growth factor; v-akt |
| | | | | AA464152; | murine thymoma viral oncogene homolog 1 |
| | | | | AA464217 | |
| 507-509 | PTEN | 322160 | Hs.10712 | U92436; | MMAC1 = PTEN = Tum or suppressor gene at |
| | | | | W37864; | 10q23.3 that is Mutated in Multiple |
| | | | | W37855 | Advanced Cancers = Phosphatase |
| | | | | | and tensin homolog |
| 510-512 | UBL1 | 758495 | Hs.81424 | U83117; | ubiquitin-homology domain protein PIC1 |
| | | | | AA401634; | |
| | | | | AA401864 | |
| 513-514 | WNT2 | 302286 | Hs.89791 | N78828; | wingless-type MMTV integration site family |
| | | | | W17194 | member 2 |
| 515-517 | SFRP4 | 841282 | Hs.105700 | AF026692; | frizzled related protein frpHE |
| | | | | AA487193; | |
| | | | | AA486838 | |
| 518-520 | RUNX1 | 263251 | Hs.129914 | D43968; | AML1 Proto-oncogene |
| | | | | H99599; | |
| | | | | H99598 | |
| 521-523 | TAL1 | 71727 | Hs.73828 | X51990; | T-cell acute lymphocytic leukemia 1 |
| | | | | T51236; | |
| | | | | T51350 | |
| 524-526 | WAS | 236282 | Hs.2157 | U12707; | Wiskott-Aldrich syndrome protein |
| | | | | H61193; | |
| | | | | H62098 | |
| 527-528 | PCTK1 | 713382 | Hs.171834 | X66363; | PCTAIRE 1 serine/threonine protein kinase |
| | | | | AA283125 | |
| 529 | EBP | 295986 | Hs.75105 | N67038 | emopamil-binding protein (sterol isomerase) |
| 530 | SMC1L1 | 897997 | Hs.211602 | AA598887 | SMC1 (structural maintenance of chromosomes 1, |
| | | | | | yeast)-like 1 |
| 531-532 | ARAF1 | 207618 | Hs.77183 | H59758; | v-raf murine sarcoma 3611 viral oncogene |
| | | | | H59757 | homolog 1 |
| 533 | UBE1 | 898262 | Hs.2055 | AA598670 | ubiquitin-activating enzyme E1 |
| 534-535 | LOC51760 | 52226 | Hs.26971 | H23265; | B/K protein |
| | | | | H23376 | |
| 536-537 | LRPAP1 | 842785 | Hs.75140 | AA486209; | low density lipoprotein-related |
| | | | | AA486313 | protein-associated protein 1 (alpha-2- |
| | | | | | macroglobulin receptor-associated |
| | | | | | protein 1) |
| 538-540 | PSTPIP1 | 71434 | Hs.129758 | L07633; | interferon-gamma IEF SSP 5111; Interferon |
| | | | | T47815; | gamma upregulated protein |
| | | | | T47814 | |
| 541-542 | IDH2 | 869375 | Hs.5337 | X69433; | isocitrate dehydrogenase 2 (NADP+), |
| | | | | AA679907 | mitochondrial |
| 543 | SIAHBP1 | 854696 | Hs.74562 | AA630094 | fuse-binding protein-interacting repressor |
| 544 | SLC25A11 | 878413 | Hs.184877 | AA670357 | solute carrier family 25 (mitochondrial |
| | | | | | carrier; oxoglutarate carrier), member 11 |
| 545-547 | LCN2 | 302127 | Hs.204238 | X99133; | lipocalin 2 (oncogene 24p3) |
| | | | | N79823; | |
| | | | | W38398 | |
| 548-550 | TCEB2 | 52162 | Hs.172772 | L42856; | Elongin B = RNA polymerase II |
| | | | | H22966; | transcription factor SIII p18 subunit |
| | | | | H24146 | |
| 551-553 | NM23H1 | 176482 | | X17620; | nm23-H1 = NDP kinase A = Nucleoside |
| | | | | H42520; | dephophate kinase A |
| | | | | H43520 | |
| 554-556 | SCYB5 | 198699 | Hs.89714 | X78686; | ENA78 = chemokine |
| | | | | R95077; | |
| | | | | R95145 | |
| 557-558 | PAK2 | 231951 | Hs.284275 | U25975; | hPAK65 = SER/THR-protein kinase PAK- |
| | | | | H92785 | gamma = P21-activated kinase 3 |
| 559-561 | S100A4 | 472180 | Hs.81256 | M80563; | S100 calcium binding protein A4 = Placental |
| | | | | AA057375; | calcium binding protein = Calvasculin = |
| | | | | AA036758 | mts1 PROTEIN = CAPL |
| 562-564 | CD83 | 564503 | Hs.79197 | Z11697; | CD83 = B-G antigen IgV domain |
| | | | | AA101749; | homolog = B-cell activation |
| | | | | AA101748 | protein = HB15 |
| 565-567 | NCOA1 | 609445 | Hs.74002 | U59302; | SRC-1 = steroid receptor coactivator |
| | | | | AA180462; | |
| | | | | AA179970 | |
| 568-570 | RYBP | 649654 | Hs.7910 | AF179286; | Death effector domain-associated |
| | | | | AA216739; | factor = Binds to Caspase 10 DED |
| | | | | AA216519 | domain = Homolog of mouse RYBP |
| | | | | | repressor protein that interacts with |
| | | | | | Polycomb complex and YY1 = YAF2 |
| | | | | | homolog = DEDAF = YAF2 |
| | | | | | homolog = MLNewGene3 |
| 571-573 | ITGAE | 665279 | Hs.851 | L25851; | CD103 alpha = Integrin alpha-E |
| | | | | AA195282; | |
| | | | | AA195146 | |
| 574-576 | IL7 | 701422 | Hs.72927 | J04156; | IL-7 |
| | | | | AA287945; | |
| | | | | AA288010 | |
| 577-579 | CD36 | 243816 | Hs.75613 | M98399; | CD36 |
| | | | | N39161; | |
| | | | | N45238 | |
| 580-582 | PDGFRB | 773439 | Hs.76144 | J03278; | Platelet-derived growth factor receptor, |
| | | | | AA426020; | beta polypeptide = fused to TEL in |
| | | | | AA428115 | t(5; 12)(q33; p13) chronic myelomonocytic |
| | | | | | leukemia |
| 583-584 | IL17R | 842122 | Hs.129751 | U58917; | IL-17 receptor |
| | | | | AA634809 | |
| 585-586 | HGF | 1219612 | Hs.809 | X16323; | Hepatocyte growth factor (hepapoietin A; |
| | | | | AA687773 | scatter factor) |
| 587-588 | BAD | 1286754 | Hs.76366 | U66879; | BAD = bbc6 = proapopto tic Bcl-2 homolog |
| | | | | AA740876 | |
| 589-591 | ZNF173 | 755176 | Hs.1287 | U09825; | acid finger protein |
| | | | | AA421953; | |
| | | | | AA421952 | |
| 592-593 | ZFP161 | 285742 | Hs.156000 | D89859; | ZF5 = POZ domain zinc finger protein |
| | | | | N64141 | |
| 594-596 | RGS16 | 470132 | Hs.183601 | U70426; | A28-RGS14p = G protein signaling |
| | | | | AA029960; | regulator |
| | | | | AA029959 | |
| 597-599 | PPP1CB | 485729 | Hs.21537 | X80910; | PPP1CB = Protein phosphatase 1, |
| | | | | AA040285; | catalytic subunit, beta isoform |
| | | | | AA040284 | |
| 600-602 | GART | 502761 | Hs.82285 | X54199; | Phosphoribosylglycinamide formyltransferase |
| | | | | AA126256; | ,phospho-ribosylglycinamide synthetase, |
| | | | | AA126360 | phosphoribosylaminoi midazole synthetase |
| 603-605 | ENPP1 | 786041 | Hs.11951 | D12485; | PC-1 = alkaline nucleotide |
| | | | | AA448639; | pyrophosphatase |
| | | | | AA448731 | |
| 606-608 | MMP13 | 786029 | Hs.2936 | X75308; | MMP-13 = Matrix metalloproteinase |
| | | | | AA448634; | 13 = CL-3 = Collagenase 3 |
| | | | | AA448726 | |
| 609-611 | ILK | 292313 | Hs.6196 | U40282; | ILK = integrin-linked kinase |
| | | | | N62542; | |
| | | | | N79210 | |
| 612-614 | SCYA4 | 205633 | Hs.75703 | J04130; | MIP-1 beta = SCAY2 = |
| | | | | H62864; | G-26 = HC21 = pAT 744 = LAG-1 = |
| | | | | H62985 | Act-2 = H400 = SIS-gamma = chemokine |
| 615-617 | KDR | 469345 | Hs.12337 | AF035121; | Kinase insert domain receptor (a type III |
| | | | | AA027012; | receptor tyrosine kinase) |
| | | | | AA026831 | |
| 618-620 | IL18R1 | 755054 | Hs.159301 | U43672; | IL-18 receptor 1 = IL-1Rrp |
| | | | | AI821652; | |
| | | | | AI734039 | |
| 621-623 | PPP2R5A | 41356 | Hs.155079 | L42373; | phosphatase 2A B56-alpha (PP2A) |
| | | | | R59165; | |
| | | | | R59164 | |
| 624-625 | PTK2B | 180298 | Hs.20313 | U43522; | protein tyrosine kinase PYK2 |
| | | | | R85257 | |
| 626-628 | MAP2K3 | 45641 | Hs.180533 | D87116; | Dual specificity mitogen-activated |
| | | | | H08749; | protein kinase kinase 3 |
| | | | | H08467 | |
| 629-631 | TNFR2RP | 124034 | Hs.117847 | L04270; | Lymphotoxin-Beta receptor precursor = |
| | | | | R02558; | Tumor necrosis factor receptor 2 related |
| | | | | R02676 | protein = Tumor necrosis factor C receptor |
| 632-633 | EST | 739852 | Hs.328687 | AI821550; | ESTs, Moderately similar to ALU4_HUMAN ALU |
| | | | | AA477842 | subfamily SB2 sequence contamination warning |
| | | | | | entry [H. sapiens] |
| 634-635 | EST | 1862171 | Hs.310541 | AI053777; | ESTs |
| | | | | AI792563 | |
| 636 | EST | 1985026 | | AI251605 | Unknown |
| 637 | EST | 2002071 | Hs.203960 | AI249848 | ESTs |
| 638 | EST | 2047317 | | AI311297 | Unknown |
| 639 | EST | 2215752 | | AI567814 | Unknown |
| 640 | EST | 2217459 | | AI744181 | Unknown |
| 641 | EST | 2217834 | Hs.328451 | AI744330 | EST, Weakly similar to PRPP_HUMAN |
| | | | | | salivary proline-rich protein II-1 |
| | | | | | [H. sapiens] |
| 642 | EST | 2219300 | | AI745684 | Unknown |
| 643 | EST | 2220085 | Hs.337231 | AI798317 | EST |
| 644 | EST | 2220214 | | AI798385 | Unknown |
| 645 | EST | 2261174 | Hs.185554 | AI609326 | EST |
| 646 | EST | 2261169 | | AI609331 | Unknown |
| 647 | EST | 2292810 | Hs.224732 | AI871658 | EST |
| 648 | EST | 2292831 | Hs.337311 | AI871678 | EST |
| 649 | EST | 2549950 | | AI954130 | Unknown |
| 650 | EST | 2550130 | | AI953438 | Unknown |
| 651-652 | FDFT1 | 25725 | Hs.48876 | R36960; | farnesyl-diphosphate farnesyltransferase 1 |
| | | | | R11842 | |
| 653-654 | NAGA | 28985 | Hs.75372 | R40255; | N-acetylgalactosaminidase, alpha- |
| | | | | R14305 | |
| 655-656 | SECRET | 29054 | Hs.116428 | R40850; | secretagogin |
| | | | | R14422 | |
| 657-658 | SLC9A1 | 30272 | Hs.170222 | R42414; | solute carrier family 9 (sodium/hydrogen |
| | | | | R14692 | exchanger), isoform 1 (antiporter, Na+/H+, |
| | | | | | amiloride sensitive) |
| 659 | TUFM | 34945 | Hs.12084 | R45183 | Tu translation elongation factor, |
| | | | | | mitochondrial |
| 660-661 | MNAT1 | 38471 | Hs.82380 | R49475; | menage a trois 1 (CAK assembly factor) |
| | | | | R35961 | |
| 662-663 | HARS | 43021 | Hs.77798 | R60150; | histidyl-tRNA synthetase |
| | | | | R60149 | |
| 664-665 | EIF4A1 | 46171 | Hs.129673 | H09590; | eukaryotic translation initiation factor 4A, |
| | | | | H09589 | isoform 1 |
| 666-667 | MPI | 50359 | Hs.75694 | HI7096; | mannose phosphate isomerase |
| | | | | H17714 | |
| 668-669 | TAGLN2 | 45544 | Hs.75725 | H08564; | transgelin 2 |
| | | | | H08563 | |
| 670-671 | PON1 | 128143 | Hs.1898 | R12373; | paraoxonase 1 |
| | | | | R09781 | |
| 672-673 | EST | 813444 | Hs.178379 | AA455945; | ESTs |
| | | | | AA455554 | |
| 674-675 | GNB2 | 292213 | Hs.91299 | N68166; | guanine nucleotide binding protein |
| | | | | N80625 | (G protein), beta polypeptide 2 |
| 676-677 | KIAA0365 | 811029 | Hs.190452 | AA485383; | KIAA0365 gene product |
| | | | | AA485539 | |
| 678 | NCSTN | 199645 | Hs.4788 | R96527 | nicastrin |
| 679-681 | ARHGEF6 | 687990 | Hs.79307 | D25304; | KIAA0006 |
| | | | | AA236957; | |
| | | | | AA236617 | |
| 682 | SFRS11 | 204755 | Hs.11482 | H56944 | splicing factor, arginine/serine-rich 11 |
| 683-684 | CUGBP1 | 25588 | Hs.81248 | R15111; | CUG triplet repeat, RNA-binding protein 1 |
| | | | | R12181 | |
| 685-686 | GABRP | 563598 | Hs.70725 | AA101225; | gamma-aminobutyric acid (GABA) A |
| | | | | AA102670 | receptor, pi |
| 687-688 | BMP6 | 768168 | Hs.285671 | AA424833; | bone morphogenetic protein 6 |
| | | | | AA426586 | |
| 689-690 | ATP7A | 687820 | Hs.606 | AA236141; | ATPase, Cu++ transporting, alpha |
| | | | | AA236635 | polypeptide (Menkes syndrome) |
| 691-692 | RBBP4 | 773599 | Hs.16003 | AA428365; | retinoblastoma-binding protein 4 |
| | | | | AA429422 | |
| 693-694 | POLR2A | 740130 | Hs.171880 | AA479052; | polymerase (RNA) II (DNA directed) |
| | | | | AA477535 | polypeptide A (220 kD) |
| 695-696 | SMG1 | 785605 | Hs.110613 | AA449463; | PI-3-kinase-related kinase SMG-1 |
| | | | | AA448998 | |
| 697-698 | GTPBP1 | 826217 | Hs.283677 | U87964; | GP-I = putative G-protein |
| | | | | AA521469 | |
| 699-700 | GS2NA | 767994 | Hs.183105 | AA418821; | nuclear autoantigen |
| | | | | AA418918 | |
| 701-702 | FLJ12442 | 32231 | Hs.84753 | R42815; | hypothetical protein FLJ12442 |
| | | | | R17469 | |
| 703-704 | KIAA0218 | 49404 | Hs.75863 | H15567; | KIAA0218 gene product |
| | | | | H15627 | |
| 705-706 | KIAA0144 | 245015 | Hs.8127 | N52646; | KIAA0144 gene product |
| | | | | N72374 | |
| 707-708 | FOXO1A | 628955 | Hs.170133 | AA194765; | forkhead box O1A (rhabdomyosarcoma) |
| | | | | AA194764 | |
| 709 | CSRP2 | 75254 | Hs.10526 | T59334 | cysteine and glycine-rich protein 2 |
| 710-711 | BRE | 739993 | Hs.80426 | AA479741; | brain and reproductive organ-expressed |
| | | | | AA477082 | (TNFRSF1A modulator) |
| 712-713 | RALY | 825583 | Hs.74111 | AA504617; | RNA-binding protein (autoantigenic) |
| | | | | AA504712 | |
| 714-716 | FGFR2 | 809464 | Hs.282823 | M87771; | FGFR2 = Fibroblast growth factor |
| | | | | AA443093; | receptor 2 |
| | | | | AA456160 | |
| 717-718 | EST | 242820 | Hs.290870 | H94050; | ESTs, Weakly similar to I38588 reverse |
| | | | | H94131 | transcriptase homolog [H. sapiens] |
| 719-720 | PEF | 137353 | Hs.241531 | R38031; | PEF protein with a long N-terminal |
| | | | | R38117 | hydrophobic domain (peflin) |
| 721-722 | EST | 265494 | Hs.153445 | N21309; | Human mRNA for unknown product, |
| | | | | N31244 | partial cds |
| 723-724 | SAST | 739625 | Hs.227489 | AA479623; | syntrophin associated serine/threonine kinase |
| | | | | AA477008 | |
| 725-726 | EST | 142499 | | R70037; | Unknown |
| | | | | R70084 | |
| 727-728 | PLXNA2 | 303035 | Hs.300622 | N91580; | plexin A2 |
| | | | | W19130 | |
| 729-730 | EST | 240694 | Hs.167787 | H78135; | ESTs |
| | | | | H78134 | |
| 731-732 | APMCF1 | 198904 | Hs.12152 | R95693; | APMCF1 protein |
| | | | | R95692 | |
| 375 | CALU | 144881 | Hs.7753 | | calumenin |
| 733-734 | PPY2 | 210873 | Hs.20588 | H67736; | pancreatic polypeptide 2 |
| | | | | H66312 | |
| 735-736 | CRB1 | 248485 | Hs.169745 | N59646; | crumbs (Drosophila) homolog 1 |
| | | | | N78199 | |
| 737-738 | FLJ21661 | 80095 | Hs.334718 | T63321; | hypothetical protein FLJ21661 |
| | | | | T63940 | |
| 739-740 | RAB3A | 163579 | Hs.27744 | H14231; | RAB3A, member RAS oncogene family |
| | | | | H14230 | |
| 741-742 | GCAT | 307094 | Hs.54609 | N93695; | glycine C-acetyltransferase (2-amino-3- |
| | | | | W21033 | ketobutyrate coenzyme A ligase) |
| 743-744 | P14L | 809437 | Hs.178576 | AA458464; | similar to Bos taurus P14 protein |
| | | | | AA442976 | |
| 745-746 | KIAA0008 | 357373 | Hs.77695 | W93717; | KIAA0008 gene product |
| | | | | W93568 | |
| 747-748 | LOX | 341680 | Hs.102267 | W60414; | lysyl oxidase |
| | | | | W60413 | |
| 749-750 | PISD | 343609 | Hs.8128 | W69460; | phosphatidylserine decarboxylase |
| | | | | W69544 | |
| 751-752 | EST | 341834 | Hs.27278 | W60647; | ESTs, Weakly similar to A Chain A, |
| | | | | W60905 | Cyclophilin A [H. sapiens] |
| 753-754 | EST | 809490 | Hs.3737 | AA443117; | ESTs |
| | | | | AA456181 | |
| 755-756 | TMEPAI | 809824 | Hs.83883 | AA455519; | transmembrane, prostate androgen |
| | | | | AA464401 | induced RNA |
| 757-758 | ZNF211 | 346947 | Hs.15110 | W79396; | zinc finger protein 211 |
| | | | | W79316 | |
| 759 | LOC51605 | 810343 | Hs.128791 | AA464166 | CGI-09 protein |
| 760-761 | MAPRE1 | 428223 | Hs.234279 | AA001749; | microtubule-associated protein, |
| | | | | AA001819 | RP/EB family, member 1 |
| 762 | FLJ10701 | 430068 | Hs.146589 | AA009830 | hypothetical protein FLJ10701 |
| 763-764 | DKFZP564C186 | 366353 | Hs.134200 | AA026278; | DKFZP564C186 protein |
| | | | | AA026277 | |
| 765 | EST | 810205 | Hs.264606 | AA464518 | ESTs |
| 766-767 | F23149_1 | 428507 | Hs.152894 | AA004525; | hypothetical protein F23149_1 |
| | | | | AA004607 | |
| 768-769 | FLJ21940 | 810795 | Hs.104916 | AA458876; | hypothetical protein FLJ21940 |
| | | | | AA459066 | |
| 770-771 | FLJ22059 | 292223 | Hs.13323 | N62464; | hypothetical protein FLJ22059 |
| | | | | N79183 | |
| 772-773 | EST | 241861 | Hs.269020 | H93115; | ESTs |
| | | | | H93243 | |
| 398 | RGS1 | 361323 | Hs.75256 | | regulator of G-protein signalling 1 |
| 774 | PDE6A | 361840 | Hs.182240 | W92514 | phosphodiesterase 6A, cGMP-specific, rod, |
| | | | | | alpha |
| 775-776 | IL1B | 491763 | Hs.126256 | AA150507; | interleukin 1, beta |
| | | | | AA156711 | |
| 777 | SF3B4 | 432564 | Hs.25797 | AA699361 | splicing factor 3b, subunit 4, 49 kD |
| 778 | EST | 277627 | Hs.348427 | N45979 | Human SH3 domain-containing protein |
| | | | | | SH3P18 mRNA, complete cds |
| 779 | TCF4 | 854581 | Hs.326198 | AA669136 | transcription factor 4 |
| 780-781 | RAB2L | 741891 | Hs.170160 | AA401972; | RAB2, member RAS oncogene family-like |
| | | | | AA402117 | |
| 782-783 | GOLGA1 | 34102 | Hs.172647 | R44140; | golgi autoantigen, golgin subfamily a, 1 |
| | | | | R23687 | |
| 784 | TNRC12 | 770000 | Hs.306094 | AA427519 | trinucleotide repeat containing 12 |
| 785 | CSNK1E | 854138 | Hs.79658 | AA669272 | casein kinase 1, epsilon |
| 786-787 | AFP | 74537 | Hs.155421 | T59043; | alpha-fetoprotein |
| | | | | T59118 | |
| 788-789 | COVA1 | 588822 | Hs.155185 | AA156560; | cytosolic ovarian carcinoma antigen 1 |
| | | | | AA157732 | |
| 790-791 | APEX | 740907 | Hs.73722 | AA478273; | APEX nuclease (multifunctional DNA |
| | | | | AA478331 | repair enzyme) |
| 792-793 | RBBP2 | 841655 | Hs.76272 | AA487492; | retinoblastoma-binding protein 2 |
| | | | | AA487706 | |
| 794-796 | GRO1 | 323238 | | M36820; | Human cytokine (GRO-beta) mRNA; |
| | | | | W42723; | GRO2 = GRO beta = MIP2 alpha = |
| | | | | W42812 | macrophage inflammatory protein- |
| | | | | | 2 alpha = chemokine |
| 797-799 | WNT2 | 149373 | | X07876; | wingless-type MMTV integration site family |
| | | | | H04382; | member 2 |
| | | | | H04408 | |
| 800 | PTGS2 | 845477 | Hs.196384 | AA644211 | cyclooxygenase-2; prostaglandin |
| | | | | | endoperoxide synthase-2 |
| 801-802 | PRNP | 470074 | Hs.74621 | AA029059; | prion protein |
| | | | | AA029163 | |
| 803-804 | WNT5B | 323636 | Hs.306051 | W44518; | Homo sapiens mRNA for WNT5B, complete |
| | | | | W44517 | cds |
| 805 | CD72 | 1241854 | | AA714696 | |
| 806 | | 897774 | | AA598510 | adenine phosphoribosyltransferase |
| 807-808 | | 795893 | | AA460168 | protein phosphatase 1, regulatory (inhibitor) |
| | | | | AA460768 | subunit 15A |
| 809-810 | | 825214 | | AA504113 | M-phase phosphoprotein 10 |
| | | | | AA504371 | |
| 811-812 | | 154720 | | R55220 | ARD1 homolog, N-acetyltransferase (S. cerevisiae) |
| | | | | R55219 | |
| 813-814 | | 204214 | | H59204 | CDC6 cell division cycle 6 homolog (S. cerevisiae) |
| | | | | H59203 | |
| 815-816 | | 815294 | | AA481547 | protein tyrosine phosphatase, receptor |
| | | | | AA481613 | type, C-associated protein |
| 817 | | 825265 | | AA504204 | polymerase (DNA directed), delta 3 |
| 818 | | 2549467 | | AI952542 | unknown EST |
| 819 | | 1056107 | | AA628360 | putative cyclin G1 interacting protein |
| 820-821 | | 809515 | | AA454565 | pLK = homologue of Drosophila polo |
| | | | | AA456458 | serine/threonine kinase |
| 822 | CMKBR6 | | | U45984 | CCR6 = STRL22 = chemokine receptor for |
| | | | | | MIP-3 alpha/LARC/Exodus on activated B cells |

TABLE 4
Markers that were Under-expressed in Ovarian Cancer in a Comparison
of Ovarian Epithelial Cancer to Normal Postmenopausal Ovarian Tissue

| | | | | Average | Average | Cancer |
|---|---|---|---|---|---|---|
| SEQ. ID. | IMAGE | Nucleic | | log | log | to |
| NO. | ID | Acid | Description | normal | cancer | normal |
| 202 | 878596 | ITM2A | integral membrane protein 2A | 1.145 | −2.036 | 0.110 |
| 203-204 | 42558 | GATM | glycine amidinotransferase | 4.137 | 0.945 | 0.109 |
| | | | (L-arginine:glycine | | | |
| | | | amidinotransferase) | | | |
| 205-207 | 81417 | RNASE4 | ribonuclease L (2′,5′- | 2.057 | −0.744 | 0.144 |
| | | | oligoisoadenylate synthetase- | | | |
| | | | dependent) | | | |
| 208-210 | 471642 | LAMA2 | laminin alpha 2 (merosin, | 2.806 | 0.361 | 0.184 |
| | | | congenital muscular dystrophy) | | | |
| 211 | 448386 | PBX3 | pre-B-cell leukemia | 2.354 | −0.243 | 0.165 |
| | | | transcription factor 3 | | | |
| 212 | 1472538 | PLA2G6 | phospholipase A2, group VI | 2.604 | 0.099 | 0.176 |
| | | | cytosolic, calcium-independent) | | | |
| 213 | 814636 | SMARCA2 | SWI/SNF related, matrix | 3.055 | 0.231 | 0.141 |
| | | | associated, actin dependent | | | |
| | | | regulator of chromatin, | | | |
| | | | subfamily a, member 2 | | | |
| 214-215 | 488956 | CUGBP2 | CUG triplet repeat, RNA- | 2.960 | −0.043 | 0.125 |
| | | | binding protein 2 | | | |
| 216-218 | 209655 | TGFBR3 | TGF beta receptor type III | 1.956 | 0.057 | 0.268 |
| 219 | 859858 | STAR | steroidogenic acute regulatory | 1.685 | 0.026 | 0.317 |
| | | | protein | | | |
| 220 | 1636447 | GNG11 | guanine nucleotide binding | 1.683 | −0.953 | 0.161 |
| | | | protein 11 | | | |
| 221 | 491565 | CITED2 | Cbp/p300-interacting | 1.576 | −0.497 | 0.238 |
| | | | transactivator, with Glu/Asp- | | | |
| | | | rich carboxy-terminal domain, 2 | | | |
| 222 | 744647 | CTNNAL1 | catenin (cadherin-associated | 1.498 | −0.761 | 0.209 |
| | | | protein), alpha-like 1 | | | |
| 223 | 743773 | ABCA8 | ATP-binding cassette, sub- | 2.317 | 0.060 | 0.209 |
| | | | family A (ABC1), member 8 | | | |
| 224-226 | 188232 | KLF4 | GKLF = EZF = KLF4 = gut- | 1.644 | −0.741 | 0.191 |
| | | | enriched Kruppel-like zinc | | | |
| | | | finger protein = expressed in | | | |
| | | | vascular endothelial cells | | | |
| 227-228 | 471725 | ITPR1 | inositol 1,4,5-triphosphate | 1.600 | −0.616 | 0.215 |
| | | | receptor, type 1 | | | |
| 229-230 | 487793 | MAF | v-maf musculoaponeurotic | 0.765 | −2.032 | 0.144 |
| | | | fibrosarcoma (avian) oncogene | | | |
| | | | homolog | | | |
| 231-232 | 768370 | FOXC1 | forkhead box C1 | 2.270 | −0.021 | 0.204 |
| 233 | 461351 | TCF21 | transcription factor 21 | 1.733 | 0.193 | 0.344 |
| 234-236 | 248295 | CCNI | Cyclin I | 2.460 | −0.030 | 0.178 |
| 237-238 | 209367 | DCN | decorin | 3.762 | 0.582 | 0.110 |
| 239-240 | 789049 | CBF2 | CCAAT-box-binding | 2.140 | 0.019 | 0.230 |
| | | | transcription factor | | | |
| 241-242 | 68049 | | Homo sapiens, clone | 2.074 | 0.243 | 0.281 |
| | | | IMAGE: 4183312, mRNA, | | | |
| | | | partial cds | | | |
| 122 | 81417 | RNASE4 | ribonuclease L (2′,5′- | 1.696 | −0.117 | 0.285 |
| | | | oligoisoadenylate synthetase- | | | |
| | | | dependent) | | | |
| 243 | 2094012 | SLC4A1AP | solute carrier family 4 (anion | 1.869 | 0.195 | 0.313 |
| | | | exchanger), member 1, adapter | | | |
| | | | protein | | | |
| 244-245 | 377731 | GSTM5 | glutathione S-transferase M5 | 1.558 | 0.242 | 0.402 |
| 246 | 460470 | C4BPB | complement component 4- | 0.750 | −0.851 | 0.330 |
| | | | binding protein, beta | | | |
| 247-248 | 73609 | HS3ST1 | heparan sulfate (glucosamine) | 2.017 | 0.328 | 0.310 |
| | | | 3-O-sulfotransferase 1 | | | |
| 249 | 2413955 | CDKN1C | cyclin-dependent kinase | 2.739 | 0.465 | 0.207 |
| | | | inhibitor 1C (p57, Kip2) | | | |
| 250 | 897823 | HNRPDL | heterogeneous nuclear | 1.703 | −0.229 | 0.262 |
| | | | ribonucleoprotein D-like | | | |
| 251 | 1558799 | C1RBP | cold inducible RNA-binding | 1.817 | −0.183 | 0.250 |
| | | | protein | | | |
| 252 | 2321596 | RGS2 | regulator of G-protein signalling | 1.607 | −0.290 | 0.269 |
| | | | 2, 24 kD | | | |
| 253-254 | 786607 | TCEAL1 | transcription elongation factor A | 1.737 | 0.083 | 0.318 |
| | | | (SII)-like 1 | | | |
| 255-256 | 377461 | CAV1 | caveolin 1, caveolae protein, | 0.146 | −2.506 | 0.159 |
| | | | 22 kD | | | |
| 257 | 855624 | ALDH1A1 | aldehyde dehydrogenase 1 | 2.030 | −0.068 | 0.233 |
| | | | family, member A1 | | | |
| 258-259 | 343443 | RBPMS | RNA-binding protein gene with | 1.727 | −0.240 | 0.256 |
| | | | multiple splicing | | | |
| 260-261 | 62263 | ADAMTS1 | a disintegrin-like and | 1.589 | 0.029 | 0.339 |
| | | | metalloprotease (reprolysin | | | |
| | | | type) with thrombospondin type | | | |
| | | | 1 motif, 1 | | | |
| 262-263 | 51408 | DSCR1L1 | Down syndrome critical region | 2.054 | 0.260 | 0.288 |
| | | | gene 1-like 1 | | | |
| 264 | 436121 | DLK1 | delta-like homolog (Drosophila) | 0.307 | −1.944 | 0.210 |
| 265-266 | 491113 | CDH11 | cadherin 11, type 2, OB- | 2.308 | 0.502 | 0.286 |
| | | | cadherin (osteoblast) | | | |
| 139 | 840776 | SGK | sgk = putative serine/threonine | 1.444 | −0.201 | 0.320 |
| | | | protein kinase transcriptionally | | | |
| | | | modified during anisotonic and | | | |
| | | | isotonic alterations of cell volume | | | |
| 267 | 450060 | HFL1 | H factor (complement)-like 1 | 2.490 | 0.664 | 0.282 |
| 268-269 | 38347 | FOG2 | Friend of GATA2 | 1.905 | 0.463 | 0.368 |
| 270 | 898122 | C7 | complement component 7 | 2.643 | 0.660 | 0.253 |
| 271-272 | 784109 | SGCE | sarcoglycan, epsilon | 1.573 | −0.213 | 0.290 |
| 273 | 898305 | NBL1 | neuroblastoma, suppression of | 1.910 | 0.153 | 0.296 |
| | | | tumorigenicity 1 | | | |
| 274-275 | 173385 | HBB | hemoglobin, beta | 0.165 | −2.317 | 0.179 |
| 276-278 | 840683 | CARP | Cytokine inducible nuclear | 1.728 | 0.018 | 0.306 |
| | | | protein | | | |
| 279-280 | 278570 | MITF | microphthalmia-associated | 0.242 | −1.580 | 0.283 |
| | | | transcription factor | | | |
| 281-282 | 898062 | CDC20 | p55CDC | 1.679 | −0.043 | 0.303 |
| 283-285 | 148028 | EPS8 | epidermal growth factor receptor | 1.595 | −0.344 | 0.261 |
| | | | kinase substrate (Eps8) | | | |
| 286-287 | 345680 | ARHI | ras homolog gene family, | 1.595 | 0.212 | 0.383 |
| | | | member 1 | | | |
| 288 | 857002 | B4-2 | proline-rich protein with nuclear | 1.523 | 0.285 | 0.424 |
| | | | targeting signal | | | |
| 289-291 | 186132 | SELE | ELAM1 = endothelial leukocyte | 1.480 | −0.393 | 0.273 |
| | | | adhesion molecule 1 | | | |
| 292-293 | 133273 | PMP22 | peripheral myelin protein 22 | 1.767 | 0.017 | 0.297 |
| 294 | 340657 | EBAF | endometrial bleeding associated | 1.422 | −0.076 | 0.354 |
| | | | factor (left-right determination, | | | |
| | | | factor A; transforming growth | | | |
| | | | factor beta superfamily) | | | |
| 295-296 | 609663 | PRKAR2B | protein kinase, cAMP-dependent, | 1.357 | −0.461 | 0.284 |
| | | | regulatory, type II, beta | | | |
| 297 | 1573311 | NFKB1E | nuclear factor of kappa light | 1.242 | −1.102 | 0.197 |
| | | | polypeptide gene enhancer in B- | | | |
| | | | cells inhibitor, epsilon | | | |
| 298-299 | 269806 | KIT | v-kit Hardy-Zuckerman 4 feline | 2.324 | 0.315 | 0.248 |
| | | | sarcoma viral oncogene homolog | | | |
| 158 | 309864 | JUNB | jun B proto-oncogene | 3.465 | 1.117 | 0.196 |
| 300 | 1573108 | BCKDK | branched chain alpha-ketoacid | 1.306 | −1.057 | 0.194 |
| | | | dehydrogenase kinase | | | |
| 301-303 | 298268 | BTG1 | BTG1 = B-cell translocation gene | 1.409 | −0.239 | 0.319 |
| | | | 1 = anti-proliferative | | | |
| 304-305 | 784772 | AKAP12 | A kinase (PRKA) anchor protein | 1.568 | −0.413 | 0.253 |
| | | | (gravin) 12 | | | |
| 306-307 | 898221 | NR4A2 | NOT = Immediate early response | 2.092 | 0.341 | 0.297 |
| | | | protein = Nurr1 | | | |
| | | | homologue = Nurr77 orphan | | | |
| | | | steroid receptor family member | | | |
| 308-309 | 126531 | HBB | hemoglobin, beta | 0.190 | −2.034 | 0.214 |
| 310-311 | 768489 | ARHGAP6 | Rho GTPase activating protein 6 | 1.263 | −0.109 | 0.386 |
| 312 | 1568391 | PLS3 | plastin 3 (T isoform) | 1.241 | −1.045 | 0.205 |
| 313-314 | 300015 | FNTA | farnesyltransferase, CAAX box, alpha | 1.354 | 0.091 | 0.417 |
| 315-316 | 770670 | TNFAIP3 | tumor necrosis factor, alpha- | 2.088 | 0.445 | 0.320 |
| | | | induced protein 3 | | | |
| 317-318 | 840944 | EGR1 | early growth response 1 | 3.245 | 0.765 | 0.179 |
| 319 | 795213 | RNAC | RNA cyclase homolog | 1.812 | 0.043 | 0.293 |
| 320 | 813584 | PA26 | p53 regulated PA26 nuclear protein | 1.329 | 0.084 | 0.422 |
| 321 | 1573778 | C11orf13 | chromosome 11 open reading | 1.241 | −1.048 | 0.205 |
| | | | frame 13 | | | |
| 322 | 2169465 | ING1L | inhibitor of growth family, | 1.267 | 0.031 | 0.425 |
| | | | member 1-like | | | |
| 323 | 2577249 | RPL9 | ribosomal protein L9 | 1.756 | −0.313 | 0.238 |
| 324-325 | 813711 | ADH5 | alcohol dehydrogenase 5 (class | 1.211 | −0.616 | 0.282 |
| | | | III), chi polypeptide | | | |
| 326-327 | 298122 | FZD7 | frizzled (Drosophila) homolog 7 | 2.554 | 0.747 | 0.286 |
| 328-329 | 366100 | MATN2 | matrilin 2 | 2.205 | 0.536 | 0.315 |
| 330-331 | 71863 | SLC11A3 | solute carrier family 11 (proton- | 2.466 | 0.808 | 0.317 |
| | | | coupled divalent metal ion | | | |
| | | | transporters), member 3 | | | |
| 332-333 | 767641 | | ESTs | 0.965 | −0.153 | 0.461 |
| 334-335 | 292463 | ERCC5 | excision repair cross- | 1.695 | −0.098 | 0.289 |
| | | | complementing rodent repair | | | |
| | | | deficiency, complementation | | | |
| | | | group 5 (xeroderma | | | |
| | | | igmentosum, complementation | | | |
| | | | group G (Cockayne syndrome)) | | | |
| 336-337 | 43771 | MGC2479 | hypothetical protein MGC2479 | 1.374 | −0.243 | 0.326 |
| 338-339 | 810617 | RPL21 | ribosomal protein L21 | 1.374 | −0.370 | 0.298 |
| 449-451 | IL8 | 328692 | interleukin 8 | | −4.52 | |
| 452-453 | GRO1 | 324437 | GRO1 oncogene (melanoma | | −3.79 | |
| | | | growth stimulating activity, | | | |
| | | | alpha) | | | |
| 454-456 | ALDH1A3 | 272686 | aldehyde dehydrogenase 1 | | −3.98 | |
| | | | family, member A3; Aldehyde | | | |
| | | | dehydrogenase 6 | | | |
| 457-459 | MMP1 | 624924 | matrix metalloproteinase 1 | | −3.72 | |
| | | | (interstitial collagenase) | | | |
| 460-461 | OSF-2 | 897910 | osteoblast specific factor 2 | | −3.12 | |
| | | | (fasciclin I-like) | | | |
| 462-464 | CDC25B | 48398 | cell division cycle 25B; M- | | −3.09 | |
| | | | phase inducer phosphatase 2 | | | |
| 465-467 | FLNA | 487418 | filamin A, alpha (actin-binding | | −3.04 | |
| | | | protein-280) | | | |
| 468-469 | TFP12 | 726086 | tissue factor pathway inhibitor 2 | | −2.82 | |
| 470-472 | FGF2 | 23073 | fibroblast growth factor 2 (basic) | | −2.72 | |
| 473-475 | CD44 | 713145 | CD44 antigen; extracellular | | −2.66 | |
| | | | matrix receptor-III = Hyaluronate | | | |
| | | | receptor | | | |
| 476-477 | DYT1 | 69046 | dystonia 1, torsion (autosomal | | −2.57 | |
| | | | dominant; torsin A) | | | |
| 478 | UCHL1 | 878833 | ubiquitin carboxyl-terminal | | −2.53 | |
| | | | esterase L1 (ubiquitin | | | |
| | | | thiolesterase) | | | |
| 479-480 | PLAU | 1696513 | plasminogen activator, urokinase | | −2.49 | |
| 256 | LDHA | 43550 | lactate dehydrogenase A | | −2.48 | |
| 481-483 | PTGS2 | 147050 | cyclooxygenase-2; prostaglandin | | −2.47 | |
| | | | endoperoxide synthase-2 | | | |
| 484-486 | PRNP | 682013 | prion protein | | 2.42; | |
| | | | | | −2.36 | |
| 487-488 | MT1X | 297392 | metallothionein 1L, | | −2.35 | |
| | | | metallothionein 1X | | | |
| 489-490 | UGB | 81336 | uteroglobin | | −2.32 | |
| 491-493 | PBEF | 594539 | pre-B-cell colony-enhancing | | −2.31 | |
| | | | factor | | | |
| 494-496 | TXNRD1 | 789376 | thioredoxin reductase 1; GRIM-12 | | −2.24 | |
| 497-499 | NT5 | 21655 | 5′ nucleotidase (CD73) | | −2.21 | |
| 500-502 | MT2A | 590150 | metallothionein 2A | | −2.21 | |
| 503 | ZNF220 | 949928 | zinc finger protein 220 | | −2.20 | |

TABLE 5
Markers that were Over-expressed in Ovarian Cancer in a Comparison of
Ovarian Epithelial Cancer to Normal Postmenopausal Ovarian Tissue

| | | | | Average | Average | Cancer |
|---|---|---|---|---|---|---|
| SEQ. ID. | | Nucleic | | log | log | to |
| NO. | IMAGE ID | Acid | Description | normal | cancer | normal |
| 18-19 | 82195 | SERPINF2 | Branched chain keto acid | | 1.45 | |
| | | | dehydrogenase E1, beta | | | |
| | | | polypeptide (maple syrup urine | | | |
| | | | disease) | | | |
| 30-31 | 295939 | FLJ22174 | hypothetical protein FLJ22174 | | 1.38 | |
| 50-51 | 755599 | IFITM1 | Interferon induced | | 1.74 | |
| | | | transmembrane protein 1 (9-27) | | | |
| 55-57 | 624655 | IFITM2 | Interferon-induced protein 1-8D | | 1.53 | |
| 60 | 786675 | HE4 | Epididymis-specific, whey-acidic | | 2.41 | |
| | | | protein type, four-disulfide core; | | | |
| | | | putative ovarian carcinoma marker | | | |
| 68-69 | 782513 | G1P3 | Interferon, alpha-inducible | | 1.64 | |
| | | | protein (clone IFI-6-16) | | | |
| 74-76 | 182288 | DDR1 | Receptor protein-tyrosine kinase | | 1.43 | |
| | | | EDDR1 | | | |
| 85-86 | 811139 | HLA-DRB5 | Major histocompatibility | | 1.91 | |
| | | | complex, class II, DR beta 5 | | | |
| 101-103 | 417711 | HLA-DRB1 | Major histocompatibility | | 1.94 | |
| | | | complex, class II, DR beta 1 | | | |
| 89-91 | 725751 | CD74 | Invariant chain = la-associated | | 2.69 | |
| | | | invariant gamma-chain | | | |
| 92-93 | 840681 | CD74 | Invariant chain = la-associated | | 2.58 | |
| | | | invariant gamma-chain | | | |
| 94-96 | 117411 | HLA-DRA | MHC Class II = DR alpha | | 1.62 | |
| 97-99 | 207715 | HLA-DPA | MHC Class II = DP alpha | | 1.85 | |
| 122-123 | 361323 | RGS1 | regulator of G-protein signaling 1 | | 1.73 | |
| 133-135 | 755279 | FOS | c-fos | | 1.76 | |
| 149-151 | 279388 | SORL1 | Mosaic protein LR11 = hybrid | | 1.56 | |
| | | | receptor gp250 precursor | | | |
| 164-166 | 813256 | ABCB1 | MDR1 = Multidrug resistance | | 1.64 | |
| | | | protein 1 = P-glycoprotein | | | |
| 167-168 | 23804 | ZFP36 | Zinc finger protein homologous | | 1.74 | |
| | | | to Zfp-36 in mouse | | | |
| 169-171 | 135880 | ZFP36 | TTP = tristetraproline = GOS24 = zin | | 2.00 | |
| | | | c finger transcriptional regulator | | | |
| 174-175 | 41650 | HGF | hepatocyte growth factor | | 1.54 | |
| | | | (hepapoietin A; scatter factor) | | | |
| 176-178 | 840776 | SGK | sgk = putative serine/threonine | | 2.02 | |
| | | | protein kinase transcriptionally | | | |
| | | | modified during anisotonic and | | | |
| | | | isotonic alteration | | | |
| 179-180 | 814508 | PPP1R7 | Protein phosphatase 1, regulatory | | 1.78 | |
| | | | subunit 7 | | | |
| 181-182 | 204335 | CD24 | CD24 antigen (small cell lung | | 2.75 | |
| | | | carcinoma cluster 4 antigen) | | | |
| 190-192 | 485770 | BRF2 | Tis 11d = ERF-2 = growth factor | | 1.62 | |
| | | | early response gene | | | |
| 199-201 | 144675 | TLR3 | TLR3 = Toll-like receptor 3 | | 1.98 | |
| 340-341 | 378813 | SLP1 | secretory leukocyte protease | −0.379 | 2.294 | 6.377 |
| | | | inhibitor (antileukoproteinase) | | | |
| 342 | 378461 | SPP1 | secreted phosphoprotein 1 | −2.657 | −0.088 | 5.938 |
| | | | (osteopontin, bone sialoprotein I, | | | |
| | | | early T-lymphocyte activation 1) | | | |
| 343-344 | 741977 | BF | B-factor, properdin | −0.362 | 1.953 | 4.974 |
| 345-347 | 810899 | CKS1 | ckshs 1 = homolog of Cks 1 = | −1.484 | 0.637 | 4.351 |
| | | | p34Cdc28/Cdc2-associated protein | | | |
| 348-349 | 470393 | MMP7 | matrix metalloproteinase 7 | 0.673 | 2.535 | 3.635 |
| | | | (matrilysin, uterine) | | | |
| 350-351 | 742101 | PAX8 | paired box gene 8 | −0.566 | 1.196 | 3.391 |
| 352-353 | 814378 | SPINT2 | serine protease inhibitor, Kunitz | −0.306 | 1.432 | 3.336 |
| | | | type, 2 | | | |
| 354 | 451907 | ZWINT | ZW10 interactor | −2.461 | −0.856 | 3.043 |
| 355 | 2544675 | DGKH | diacylglycerol kinase, eta | −0.036 | 1.498 | 2.896 |
| 356 | 782811 | HMGIY | high-mobility group (nonhistone | −2.272 | −0.760 | 2.851 |
| | | | chromosomal) protein isoforms I and Y | | | |
| 357-359 | 504763 | SDC4 | Syndecan-4 = amphiglycan = | −0.871 | 0.575 | 2.725 |
| | | | ryudocan core protein | | | |
| 360 | 1161155 | CDKN2A | cyclin-dependent kinase inhibitor | −0.839 | 0.593 | 2.699 |
| | | | 2A (melanoma, p16, inhibits CDK4) | | | |
| 361-362 | 810873 | SCNN1A | sodium channel, nonvoltage-gated 1 | 0.127 | 1.534 | 2.652 |
| | | | alpha | | | |
| 363 | 43550 | LDHA | lactate dehydrogenase A | −3.496 | −2.152 | 2.538 |
| 364-365 | 131839 | FOLR1 | folate receptor 1 (adult) | −0.867 | 0.467 | 2.522 |
| 366-367 | 855749 | TPI1 | Triosephosphate isomerase 1 | −2.272 | −1.008 | 2.400 |
| 368 | 2514426 | KLK8 | kallikrein 8 (neuropsin/ovasin) | −0.491 | 0.742 | 2.352 |
| 200 | 79629 | CXCR4 | CXC chemokine receptor 4 = | −0.588 | 0.618 | 2.307 |
| | | | fusin = neuropeptide Y | | | |
| | | | receptor = L3 | | | |
| 369-370 | 825606 | KNSL1 | kinesin-like 1 | −1.797 | −0.602 | 2.290 |
| 371-372 | 488964 | H2AFO | H2A histone family, member O | −1.329 | −0.144 | 2.274 |
| 373-374 | 153411 | HLA-DRA | major histocompatibility | 1.967 | 3.150 | 2.270 |
| | | | complex, class II, DR alpha | | | |
| 375 | 1323448 | CRIP1 | cysteine-rich protein 1 (intestinal) | 0.086 | 1.246 | 2.234 |
| 376 | 950700 | PP | pyrophosphatase (inorganic) | −1.029 | 0.118 | 2.214 |
| 377-378 | 666391 | ESTs | Unknown | 0.214 | 1.360 | 2.212 |
| 379-381 | 207358 | SLC2A1 | glucose transporter (HepG2) | −1.190 | −0.050 | 2.204 |
| 382 | 897770 | ESTs | Unknown | −0.173 | 0.943 | 2.167 |
| 383-385 | 813673 | HDGF | hepatoma-derived growth factor | −0.786 | 0.329 | 2.166 |
| 386 | 882522 | ASS | argininosuccinate synthetase | −0.424 | 0.676 | 2.143 |
| 387-388 | 770388 | CLDN4 | claudin 4 | 0.065 | 1.159 | 2.135 |
| 389 | 897956 | PRAME | preferentially expressed antigen | −2.071 | −0.977 | 2.134 |
| | | | in melanoma | | | |
| 390-391 | 897788 | PTPRF | LAR = LCA-homologue | −0.175 | 0.900 | 2.108 |
| 392-393 | 741139 | EYA2 | eyes absent (Drosophila) homolog 2 | −0.133 | 0.939 | 2.102 |
| 394-396 | 138917 | MYCL1 | L-myc | −0.042 | 1.026 | 2.096 |
| 397-399 | 840691 | STAT1 | STAT1 = IFN alpha/beta- | −0.023 | 1.044 | 2.095 |
| | | | responsive transcription factor | | | |
| | | | 1SGF3 beta subunits (p91/p84) | | | |
| 400-401 | 564492 | MTCH2 | mitochondrial carrier homolog 2 | −1.566 | −0.512 | 2.076 |
| 402 | 435597 | HTR3A | 5-hydroxytryptamine (serotonin) | −0.392 | 0.656 | 2.067 |
| | | | receptor 3A | | | |
| 403-404 | 68950 | CCNE1 | cyclin E1 | −0.470 | 0.577 | 2.066 |
| 405 | 739155 | CDH6 | cadherin 6, type 2, K-cadherin | −0.286 | 0.748 | 2.048 |
| | | | (fetal kidney) | | | |
| 406-408 | 531028 | PRKAG1 | 5′-AMP-activated protein kinase, | 0.148 | 1.181 | 2.046 |
| | | | gamma-1 subunit | | | |
| 409 | 2403485 | DEFB1 | defensin, beta 1 | 0.335 | 1.357 | 2.031 |
| 410-411 | 626502 | ARPC1B | actin related protein 2/3 complex, | −0.808 | 0.213 | 2.030 |
| | | | subunit 1A (41 kD) | | | |
| 412-414 | 71622 | PRKCI | PKC iota = Protein kinase C, iota | −0.202 | 0.802 | 2.006 |
| 415 | 1610448 | GAPD | glyceraldehyde-3-phosphate | −1.484 | −0.480 | 2.005 |
| | | | dehydrogenase | | | |
| 416-417 | 85497 | C2 | complement component 2 | −0.413 | 0.589 | 2.002 |
| 418-419 | 843075 | H2AFY | H2A histone family, member Y | −0.829 | 0.164 | 1.990 |
| 420-421 | 840567 | TM4SF1 | transmembrane 4 superfamily | −1.261 | −0.270 | 1.987 |
| | | | member 1 | | | |
| 422-423 | 50117 | GAPD | glyceraldehyde-3-phosphate | −2.692 | −1.706 | 1.981 |
| | | | dehydrogenase | | | |
| 424-426 | 809910 | IFITM3 | Interferon-inducible protein 1-8U | −0.099 | 0.887 | 1.981 |
| 427-428 | 248261 | GLDC | glycine dehydrogenase | −0.757 | 0.221 | 1.970 |
| | | | (decarboxylating; glycine | | | |
| | | | decarboxylase, glycine cleavage | | | |
| | | | system protein P) | | | |
| 429-430 | 144881 | CALU | calumenin | −1.579 | −0.620 | 1.943 |
| 431-432 | 208764 | HBA2 | hemoglobin, alpha 2 | −0.114 | 0.837 | 1.934 |
| 433 | 810612 | S100A11 | S100 calcium-binding protein | −0.670 | 0.279 | 1.931 |
| | | | A11 (calgizzarin) | | | |
| 434-436 | 897567 | LDHA | Lactate dehydrogenase A | −2.982 | −2.038 | 1.925 |
| 437 | 769921 | UBE2C | ubiquitin-conjugating enzyme | −1.429 | −0.487 | 1.922 |
| | | | E2C | | | |
| 438-440 | 304908 | E2F3 | E2F-3 = pRB-binding transcription | −0.526 | 0.416 | 1.921 |
| | | | factor = KIAA0075 | | | |
| 441-442 | 251019 | CDH1 | E-cadherin | −0.248 | 0.682 | 1.905 |
| 443-444 | 210405 | PSME2 | proteasome (prosome, | −0.715 | 0.213 | 1.902 |
| | | | macropain) activator subunit 2 | | | |
| | | | (PA28 beta) | | | |
| 445-447 | 344430 | BMP7 | OP-1 = osteogenic protein in the | −0.075 | 0.852 | 1.901 |
| | | | TGF-beta family | | | |
| 448 | 825470 | TOP2A | TOP2A | | | 2 |

TABLE 6
Markers that were Differentially Expressed Between BRCA1-Linked and Sporadic
Tumors in a Comparison to reference Immortalized Ovarian Epithelial Cells.

| | | | Geometric | Geometric | Fold |
|---|---|---|---|---|---|
| | | | mean of | mean of | difference |
| SEQ ID | Nucleic | | ratios in | ratios in | in geometric |
| NO. | Acid | Description | BRCA1 | sporadic | means |
| 805 | CD72 | B-cell differentiation antigen | 1.49 | 1.17 | 0.79 |
| | | CD72 (human); | | | |
| 544 | SLC25A11 | solute carrier family 25 | 1.27 | 1.08 | 0.84 |
| | | (mitochondrial carrier; | | | |
| | | oxoglutarate carrier), member 11 | | | |
| 545-547 | LCN2 | lipocalin 2 (oncogene 24p3) | 1.29 | 0.98 | 0.76 |
| 538-540 | PSTPIP1 | interferon-gamma IEF SSP | 1.95; | 1.31; | 0.67; |
| | | 5111; Interferon gamma | 1.6 | 1.04 | 0.65 |
| | | upregulated protein | | | |
| 543 | S1AHBP1 | fuse-binding protein-interacting | 1.86 | 1.21 | 0.65 |
| | | repressor | | | |
| 533 | UBE1 | ubiquitin-activating enzyme E1 | 1.54 | 0.94 | 0.61 |
| 524-526 | WAS | Wiskott-Aldrich syndrome protein | 1.13 | 0.79 | 0.7 |
| 541-542 | IDH2 | isocitrate dehydrogenase 2 | 1.69 | 1.02 | 0.6 |
| | | (NADP+), mitochondrial | | | |
| 527-528 | PCTK1 | PCTAIRE 1 serine/threonine | 1.33 | 1.12 | 0.84 |
| | | protein kinase | | | |

TABLE 7
Markers that were Differentially Expressed Between BRCA2-Linked and Sporadic
Tumors in a Comparison to reference Immortalized Ovarian Epithelial Cells.

| | | | Geometric | Geometric | Fold |
|---|---|---|---|---|---|
| | | | mean of | mean of | difference |
| SEQ | | | ratios in | ratios in | in |
| ID | Nucleic | | BRCA1-linked | sporadic | geometric |
| NO. | Acid | Description | tumors | tumors | means |
| 279 | LOC51760 | B/K protein | 1.32 | 1.1 | 0.83 |
| 280 | LRPAP1 | low density lipoprotein- | 1.45 | 1.13 | 0.78 |
| | | related protein-associated | | | |
| | | protein 1 (alpha-2- | | | |
| | | macroglobulin receptor- | | | |
| | | associated protein 1) | | | |

TABLE 8
Markers that were Differentially Expressed Between Combined
BRCA-Linked Group and Sporadic Tumors in a Comparison to
reference Immortalized Ovarian Epithelial Cells.

| | | | Geometric | Geometric | Fold |
|---|---|---|---|---|---|
| | | | mean of | mean of | difference |
| SEQ | | | ratios in | ratios in | in |
| ID | Nucleic | | BRCA1-linked | sporadic | geometric |
| NO. | Acid | Description | tumors | tumors | means |
| 281 | PSTPIP1 | interferon-gamma IEF SSP | 1.73; | 1.31; | 0.76; |
| | | 5111 = Interferon gamma | 1.41 | 1.04 | 0.74 |
| | | upregulated protein | | | |
| 282 | IDH2 | Isocitrate dehydrogenase 2 | 1.66 | 1.02 | 0.61 |
| | | (NADP+), mitochondrial | | | |
| 274 | PCTK1 | PCTAIRE 1 serine/threonine | 1.29 | 1.12 | 0.86 |
| | | protein kinase | | | |

TABLE 9
Markers that were Differentially Expressed between BRCA1-like and BRCA2-like
tumors in a Comparison to reference Immortalized Ovarian Epithelial Cells.

| | | | Geometric | Geometric | Fold |
|---|---|---|---|---|---|
| | | | mean of | mean of | Difference |
| SEQ ID | | | ratios in | ratios in | in Geometric |
| NO: | Gene | Description | BRCA1 | BRCA2 | Means |
| 122-123 | RGS1 | regulator of G-protein signalling 1 | 1.79 | 4.75 | 2.65 |
| 122-123 | RGS1 | BL34 = RGS1 = regulator of | 2.09 | 5.05 | 2.41 |
| | | G-protein signaling which inhibits | | | |
| | | SDF-1 directed B cell migration | | | |
| 594-596 | RGS16 | A28-RGS14p = G protein signaling | 1.22 | 2.32 | 1.9 |
| | | regulator | | | |
| 612-614 | SCYA4 | MIP-1 beta = SCAY2 = G-26 = | 1.29 | 2.23 | 1.73 |
| | | HC21 = pAT 744 = LAG-1 = Act-2 = | | | |
| | | H400 = SIS-gamma = chemokine | | | |
| 612-614 | SCYA4 | MIP-1 beta = SCAY2 = G-26 = HC21 = | 1.09 | 1.79 | 1.64 |
| | | pAT 744 = LAG-1 = Act-2 = H400 = | | | |
| | | SIS-gamma = chemokine | | | |
| 515-517 | SFRP4 | frizzled related protein frpHE | 1.13 | 1.85 | 1.63 |
| 594-596 | RGS16 | A28-RGS14p = G protein signaling | 1.33 | 2.11 | 1.58 |
| | | regulator | | | |
| 790-791 | APEX | APEX nuclease (multifunctional DNA | 0.66 | 1.04 | 1.58 |
| | | repair enzyme) | | | |
| 682 | SFRS11 | splicing factor, arginine/serine-rich 11 | 0.69 | 1.09 | 1.57 |
| 507-509 | PTEN | MMAC1 = PTEN = Tumor suppressor gene | 1.03 | 1.56 | 1.51 |
| | | at 10q23.3 that is Mutated in Multiple | | | |
| | | Advanced Cancers = Phosphatase and | | | |
| | | tensin homolog | | | |
| 774 | PDE6A | phosphodiesterase 6A, cGMP-specific, rod, | 1.23 | 1.85 | 1.51 |
| | | alpha | | | |
| 562-564 | CD83 | CD83 = B-G antigen 1gV domain homolog = | 1.46 | 2.19 | 1.5 |
| | | B-cell activation protein = HB15 | | | |
| 592-593 | ZFP161 | ZF5 = POZ domain zinc finger protein | 1.03 | 1.49 | 1.45 |
| | | ESTs | 1.17 | 1.69 | 1.44 |
| 707-708 | FOXO1A | forkhead box O1A (rhabdomyosarcoma) | 1.38 | 1.93 | 1.4 |
| 762 | FLJ10701 | hypothetical protein FLJ10701 | 1 | 1.4 | 1.39 |
| 577-579 | CD36 | CD36 | 1.32 | 1.82 | 1.38 |
| 797-799 | WNT2 | Wnt-2 | 0.81 | 1.12 | 1.38 |
| | | Unknown | 0.82 | 1.13 | 1.38 |
| 779 | TCF4 | transcription factor 4 | 1.19 | 1.62 | 1.36 |
| 615-617 | KDR | Kinase insert domain receptor (a type III | 0.8 | 1.08 | 1.35 |
| | | receptor tyrosine kinase) | | | |
| | | ESTs | 0.81 | 1.09 | 1.35 |
| 534-535 | LOC51760 | B/K protein | 0.98 | 1.32 | 1.35 |
| 797-799 | WNT2 | Wnt-2 | 0.99 | 1.33 | 1.34 |
| 683-684 | CUGBP1 | CUG triplet repeat, RNA-binding protein 1 | 0.73 | 0.98 | 1.33 |
| 709 | CSRP2 | cysteine and glycine-rich protein 2 | 0.98 | 1.31 | 1.33 |
| | | ESTs, Moderately similar to | 0.81 | 1.06 | 1.32 |
| | | ALU4_HUMAN ALU SUBFAMILY SB2 | | | |
| | | SEQUENCE CONTAMINATION WARNING | | | |
| | | ENTRY [H. sapiens] | | | |
| 606-608 | MMP13 | MMP-13 = Matrix metalloproteinase | 0.99 | 1.3 | 1.32 |
| | | 13 = CL-3 = Collagenase 3 | | | |
| 580-582 | PDGFRB | Platelet-derived growth factor receptor, | 1.41 | 1.85 | 1.31 |
| | | beta polypeptide = fused to TEL in | | | |
| | | t(5; 12)(q33; p13) chronic myelomonocytic | | | |
| | | leukemia | | | |
| 603-605 | ENPP1 | PC-1 = alkaline nucleotide pyro- | 1.01 | 1.32 | 1.31 |
| | | phosphatase | | | |
| | | FGFR2 = Fibroblast growth factor | 0.8 | 1.05 | 1.31 |
| | | receptor 2 | | | |
| 695-696 | SMG1 | PI-3-kinase-related kinase SMG-1 | 1.07 | 1.4 | 1.31 |
| 521-523 | TAL1 | scl = tal-1 = T-cell acute | 1.14 | 1.49 | 1.31 |
| | | lymphocytic leukemia 1 | | | |
| 727-728 | PLXNA2 | plexin A2 | 1.32 | 1.71 | 1.3 |
| 759 | LOC51605 | CGI-09 protein | 0.8 | 1.04 | 1.3 |
| 784 | TNRC12 | trinucleotide repeat containing 12 | 1.02 | 1.33 | 1.3 |
| | | EST | 0.86 | 1.12 | 1.3 |
| 797-799 | WNT2 | wingless-type MMTV integration site | 0.98 | 1.27 | 1.29 |
| | | family member 2 | | | |
| 693-694 | POLR2A | polymerase (RNA) II (DNA directed) | 0.79 | 1.01 | 1.29 |
| | | polypeptide A (220 kD) | | | |
| 737-738 | FLJ21661 | hypothetical protein FLJ21661 | 0.61 | 0.79 | 1.28 |
| 780-781 | RAB2L | RAB2, member RAS oncogene family-like | 1.05 | 1.35 | 1.28 |
| 577-579 | CD36 | CD36 | 1.26 | 1.61 | 1.28 |
| 568-570 | RYBP | Death effector domain-associated factor = | 0.89 | 1.14 | 1.28 |
| | | Binds to Caspase 10 DED domain = Homolog | | | |
| | | of mouse RYBP repressor protein that | | | |
| | | interacts with Polycomb complex and | | | |
| | | YY1 = YAF2 homolog = DEDAF = | | | |
| | | YAF2 homolog = MLNewGene3 | | | |
| 571-573 | ITGAE | CD103 alpha = Integrin alpha-E | 1.09 | 1.38 | 1.27 |
| | | Human SH3 domain-containing protein | 1.23 | 1.56 | 1.27 |
| | | SH3P18 mRNA, complete cds | | | |
| 755-756 | TMEPAI | transmembrane, prostate androgen | 0.99 | 1.25 | 1.26 |
| | | induced RNA | | | |
| 565-567 | NCOA1 | SRC-1 = steroid receptor coactivator | 1.04 | 1.3 | 1.25 |
| 785 | CSNK1E | casein kinase 1, epsilon | 0.74 | 0.92 | 1.25 |
| 768-769 | FLJ21940 | hypothetical protein FLJ21940 | 0.91 | 1.14 | 1.25 |
| 723-724 | SAST | syntrophin associated serine/threonine | 1.05 | 1.32 | 1.25 |
| | | kinase | | | |
| | | ESTs | 0.89 | 1.11 | 1.25 |
| 782-783 | GOLGA1 | golgi autoantigen, golgin subfamily a, 1 | 0.76 | 0.95 | 1.24 |
| 574-576 | IL7 | IL-7 | 0.99 | 1.23 | 1.24 |
| 319 | RNAC | RNA cyclase homolog | 0.92 | 1.13 | 1.24 |
| 676-677 | KIAA0365 | KIAA0365 gene product | 0.97 | 1.2 | 1.23 |
| 679-681 | ARHGEF6 | KIAA0006 | 1.04 | 1.28 | 1.23 |
| 710-711 | BRE | brain and reproductive organ-expressed | 1.11 | 1.35 | 1.22 |
| | | (TNFRSF1A modulator) | | | |
| | | Unknown | 0.9 | 1.11 | 1.22 |
| 670-671 | PON1 | paraoxonase 1 | 0.93 | 1.14 | 1.22 |
| | | ESTs, Weakly similar to I38588 reverse | 1.11 | 1.36 | 1.22 |
| | | transcriptase homolog [H. sapiens] | | | |
| | | ESTs | 0.72 | 0.87 | 1.21 |
| | ATP7A | ATPase, Cu++ transporting, alpha | 0.99 | 1.2 | 1.21 |
| | | polypeptide (Menkes syndrome) | | | |
| | | Unknown | 1.2 | 1.45 | 1.21 |
| 735-736 | CRB1 | crumbs (Drosophila) homolog 1 | 0.88 | 1.06 | 1.21 |
| 757-758 | ZNF211 | zinc finger protein 211 | 0.82 | 0.99 | 1.21 |
| | | ESTs | 0.99 | 1.16 | 1.18 |
| 685-686 | GABRP | gamma-aminobutyric acid (GABA) A | 0.91 | 1.07 | 1.17 |
| | | receptor, pi | | | |
| 687-688 | BMP6 | bone morphogenetic protein 6 | 0.95 | 1.1 | 1.16 |
| 587-588 | BAD | BAD = bbc6 = proapoptotic | 1.11 | 0.94 | 0.85 |
| | | Bcl-2 homolog | | | |
| 678 | NCSTN | nicastrin | 1.13 | 0.94 | 0.83 |
| 766-767 | F23149_1 | hypothetical protein F23149_1 | 1.11 | 0.91 | 0.82 |
| 701-702 | FLJ12442 | hypothetical protein FLJ12442 | 1.04 | 0.85 | 0.82 |
| 589-591 | ZNF173 | acid finger protein | 1.16 | 0.95 | 0.81 |
| 741-742 | GCAT | glycine C-acetyltransferase (2-amino- | 1.12 | 0.91 | 0.81 |
| | | 3-ketobutyrate coenzyme A ligase) | | | |
| 786-787 | AFP | alpha-fetoprotein | 1.2 | 0.96 | 0.8 |
| | | hPAK65 = SER/THR-protein kinase | 1.05 | 0.84 | 0.8 |
| | | PAK-gamma = P21-activated kinase 3 | | | |
| 747-748 | LOX | lysyl oxidase | 0.93 | 0.75 | 0.8 |
| 662-663 | HARS | histidyl-tRNA synthetase | 0.73 | 0.57 | 0.79 |
| 544 | SLC25A11 | solute carrier family 25 (mitochondrial | 1.27 | 1 | 0.78 |
| | | carrier; oxoglutarate carrier), member 11 | | | |
| 697-698 | GTPBP1 | GP-1 = putative G-protein | 0.97 | 0.76 | 0.78 |
| 699-700 | GS2NA | nuclear autoantigen | 0.96 | 0.75 | 0.78 |
| 705-706 | KIAA0144 | KIAA0144 gene product | 0.87 | 0.68 | 0.78 |
| | | Unknown | 1.02 | 0.79 | 0.78 |
| 733-734 | PPY2 | pancreatic polypeptide 2 | 1.49 | 1.16 | 0.78 |
| 653-654 | NAGA | N-acetylgalactosaminidase, alpha- | 1.06 | 0.82 | 0.78 |
| 583-584 | IL17R | IL-17 receptor | 1.05 | 0.82 | 0.78 |
| 657-658 | SLC9A1 | solute carrier family 9 (sodium/hydrogen | 0.97 | 0.75 | 0.77 |
| | | exchanger), isoform 1 (antiporter, | | | |
| | | Na+/H+, amiloride sensitive) | | | |
| 691-692 | RBBP4 | retinoblastoma-binding protein 4 | 0.87 | 0.67 | 0.76 |
| 609-611 | ILK | ILK = integrin-linked kinase | 0.88 | 0.67 | 0.76 |
| 624-625 | PTK2B | protein tyrosine kinase PYK2 | 1.07 | 0.81 | 0.76 |
| 504-506 | AKT1 | BPGF-1 = bone-derived growth factor = | 0.75 | 0.56 | 0.75 |
| | | Q6 = quiescin = expression is induced | | | |
| | | by reversible growth arrest, trypsinization | | | |
| | | and serum starvation and is blocked by | | | |
| | | SV40 transformation | | | |
| 763-764 | DKFZP564C186 | DKFZP564C186 protein | 0.97 | 0.73 | 0.75 |
| 554-556 | SCYB5 | ENA78 = chemokine | 0.34 | 0.25 | 0.75 |
| 660-661 | MNAT1 | menage a trois 1 (CAK assembly factor) | 1.24 | 0.93 | 0.75 |
| 548-550 | TCEB2 | Elongin B = RNA polymerase II transcription | 0.98 | 0.73 | 0.74 |
| | | factor SIII p18 subunit | | | |
| 792-793 | RBBP2 | retinoblastoma-binding protein 2 | 1.47 | 1.08 | 0.74 |
| 626-628 | MAP2K3 | Dual specificity mitogen-activated protein | 1.06 | 0.78 | 0.74 |
| | | kinase kinase 3 | | | |
| 712-713 | RALY | RNA-binding protein (autoantigenic) | 0.85 | 0.62 | 0.74 |
| 743-744 | P14L | similar to Bos taurus P14 protein | 0.87 | 0.64 | 0.73 |
| 731-732 | APMCF1 | APMCF1 protein | 0.95 | 0.7 | 0.73 |
| 674-675 | GNB2 | guanine nucleotide binding protein (G | 0.99 | 0.73 | 0.73 |
| | | protein), beta polypeptide 2 | | | |
| | | Lymphotoxin-Beta receptor precursor = | 1.28 | 0.93 | 0.73 |
| | | Tumor necrosis factor receptor 2 related | | | |
| | | protein = Tumor necrosis factor C receptor | | | |
| | | ESTs | 1.02 | 0.73 | 0.72 |
| 666-667 | MPI | mannose phosphate isomerase | 1.22 | 0.87 | 0.71 |
| 719-720 | PEF | PEF protein with a long N-terminal | 1 | 0.71 | 0.71 |
| | | hydrophobic domain (peflin) | | | |
| 651-652 | FDFT1 | farnesyl-diphosphate farnesyltransferase 1 | 1.09 | 0.77 | 0.71 |
| 739-740 | RAB3A | RAB3A, member RAS oncogene family | 0.8 | 0.57 | 0.71 |
| | | EST | 1.49 | 1.05 | 0.71 |
| 621-623 | PPP2R5A | phosphatase 2A B56-alpha (PP2A) | 0.89 | 0.63 | 0.71 |
| 600-602 | GART | Phosphoribosylglycinamide formyltransferase, | 0.82 | 0.58 | 0.7 |
| | | phosphoribosylglycinamide synthetase, | | | |
| | | phosphoribosylaminoimidazole synthetase | | | |
| 551-553 | NM23H1 | nm23-H1 = NDP kinase A = Nucleoside | 0.71 | 0.49 | 0.7 |
| | | dephophate kinase A | | | |
| 655-656 | SECRET | secretagogin | 0.84 | 0.58 | 0.69 |
| | | Unknown | 0.8 | 0.55 | 0.69 |
| | | EST | 1.19 | 0.82 | 0.69 |
| 770-771 | FLJ22059 | hypothetical protein FLJ22059 | 0.7 | 0.49 | 0.69 |
| 659 | TUFM | Tu translation elongation factor, mitochondrial | 1.16 | 0.8 | 0.69 |
| 518-520 | RUNX1 | core binding factor alpha 1b subunit = CBF | 0.69 | 0.47 | 0.69 |
| | | alpha1 = PEBP2aA1 transcription factor = | | | |
| | | AML1 Proto-oncogene = translocated in | | | |
| | | acute myeloid leukemia | | | |
| 585-586 | HGF | Hepatocyte growth factor (hepapoietin A; | 0.88 | 0.6 | 0.68 |
| | | scatter factor) | | | |
| | | EST, Weakly similar to PRPP_HUMAN SALIVARY | 0.98 | 0.67 | 0.68 |
| | | PROLINE-RICH PROTEIN II-1 [H. sapiens] | | | |
| | | Unknown | 1.02 | 0.7 | 0.68 |
| | | Human mRNA for unknown product, partial cds | 0.57 | 0.39 | 0.68 |
| 618-620 | IL18R1 | IL-18 receptor 1 = 1L-1Rrp | 0.88 | 0.6 | 0.68 |
| 510-512 | UBL1 | ubiquitin-homology domain protein PIC1 | 1.49 | 1.01 | 0.68 |
| 703-704 | KIAA0218 | KIAA0218 gene product | 1.2 | 0.81 | 0.68 |
| 760-761 | MAPRE1 | microtubule-associated protein, RP/EB family, | 0.6 | 0.4 | 0.67 |
| | | member 1 | | | |
| 777 | SF3B4 | splicing factor 3b, subunit 4, 49 kD | 1.17 | 0.78 | 0.67 |
| | | Unknown | 1.04 | 0.7 | 0.67 |
| | | ESTs | 0.42 | 0.28 | 0.66 |
| 533 | UBE1 | ubiquitin-activating enzyme E1 (A1S9T and | 1.54 | 1.01 | 0.66 |
| | | BN75 temperature sensitivity complementing) | | | |
| | | Unknown | 1.23 | 0.81 | 0.66 |
| 788-789 | COVA1 | cytosolic ovarian carcinoma antigen 1 | 0.57 | 0.37 | 0.64 |
| | | Unknown | 0.82 | 0.53 | 0.64 |
| 668-669 | TAGLN2 | transgelin 2 | 0.7 | 0.44 | 0.64 |
| 749-750 | PISD | phosphatidylserine decarboxylase | 0.8 | 0.5 | 0.62 |
| 775-776 | IL1B | interleukin 1, beta | 0.3 | 0.17 | 0.58 |
| 429-430 | CALU | calumenin | 0.7 | 0.39 | 0.56 |
| 597-599 | PPP1CB | PPP1CB = Protein phosphatase 1, catalytic | 0.78 | 0.44 | 0.56 |
| | | subunit, beta isoform | | | |
| 751-752 | | ESTs, Weakly similar to A Chain A, Cyclophilin A | 0.78 | 0.43 | 0.55 |
| | | [H. sapiens] | | | |
| 745-746 | KIAA0008 | KIAA0008 gene product | 0.49 | 0.27 | 0.55 |
| | | Unknown | 2 | 1.09 | 0.54 |
| 664-665 | EIF4A1 | eukaryotic translation initiation factor 4A, | 0.7 | 0.37 | 0.53 |
| | | isoform 1 | | | |
| 559-561 | S100A4 | S100 calcium binding protein A4 = Placental | 2.28 | 1.21 | 0.53 |
| | | calcium binding protein = Calvasculin = | | | |
| | | mts1 PROTEIN = CAPL | | | |
| 375 | PPIA | peptidylprolyl isomerase A (cyclophilin A) | 0.76 | 0.39 | 0.52 |
| | | EST | 2.56 | 1.26 | 0.49 |

TABLE 10
Markers that can be used to Classify BRCA1-like from BRCA2-like Tumor Types using Compound Covariate Prediction Analysis.

| | | | | Average | | |
|---|---|---|---|---|---|---|
| | | | | Log ratios | Midpoint of | Average log |
| | | | | in BRCA2 | average log- | ratios in |
| SEQ ID | | | | & BRCA2-like | ratios in | BRCA1&BRCA1- |
| NO: | Gene | Description | t-value | sporadics* | each class | like sporadic |
| 659 | TUFM | Tu translation elongation factor, | −10 | −0.09854 | −0.016 | 0.067443 |
| | | mitochondrial | | | | |
| 749-750 | PISD | phosphatidylserine decarboxylase | −8.2305 | −0.28567 | −0.187 | −0.08778 |
| 745-746 | KIAA0008 | KIAA0008 gene product | −8.0421 | −0.56864 | −0.431 | −0.29414 |
| 703-704 | KIAA0218 | KIAA0218 gene product | −7.9288 | −0.08197 | −0.005 | 0.071882 |
| 751-752 | EST | ESTs, Weakly similar to A Chain A, | −7.6225 | −0.34775 | −0.225 | −0.10292 |
| | | Cyclophilin A [H. sapiens] | | | | |
| 621-623 | PPP2R5A | phosphatase 2A B56-alpha (PP2A) | −7.469 | −0.20343 | −0.121 | −0.03763 |
| 733-734 | PPY2 | pancreatic polypeptide 2 | −7.3866 | 0.06558 | 0.113 | 0.160168 |
| 649 | EST | Unknown | −7.384 | −0.27327 | −0.183 | −0.09313 |
| 641 | EST | EST, Weakly similar to PRPP_HUMAN | −7.3561 | −0.17457 | −0.095 | −0.01592 |
| | | SALIVARY PROLINE-RICH PROTEIN II-1 | | | | |
| | | [H. sapiens] | | | | |
| 375 | PPIA | peptidylprolyl isomerase A | −6.9946 | −0.38934 | −0.258 | −0.12668 |
| | | (cyclophilin A) | | | | |
| 770-771 | FLJ22059 | hypothetical protein FLJ22059 | −6.9726 | −0.31605 | −0.228 | −0.14026 |
| 739 | RAB3A | RAB3A, member RAS oncogene family | −6.9458 | −0.23582 | −0.167 | −0.098 |
| 655-656 | SECRET | secretagogin | −6.9307 | −0.23657 | −0.147 | −0.0575 |
| 629-631 | TNFR2RP | Lymphotoxin-Beta receptor precursor = | −6.9268 | −0.02733 | 0.038 | 0.103462 |
| | | Tumor necrosis factor receptor 2 related | | | | |
| | | protein = Tumor necrosis factor C | | | | |
| | | receptor | | | | |
| 551-553 | NM23H1 | nm23-H1 = NDP kinase A = Nucleoside | −6.8307 | −0.32239 | −0.242 | −0.16241 |
| | | dephophate kinase A | | | | |
| 557-558 | PAK2 | hPAK65 = SER/THR-protein kinase PAK- | −6.7214 | −0.1152 | −0.05 | 0.01536 |
| | | gamma = P21-activated kinase 3 | | | | |
| 806 | APRT | adenine phosphoribosyltransferase | −6.6725 | 0.044932 | 0.085 | 0.125156 |
| 807-808 | PPP1R15A | protein phosphatase 1, regulatory | −6.648 | −0.21681 | −0.144 | −0.0716 |
| | | (inhibitor) subunit 15A | | | | |
| 544 | SLC25A11 | solute carrier family 25 (mitochondrial | −6.6083 | 0.003461 | 0.047 | 0.089905 |
| | | carrier; oxoglutarate carrier), member 11 | | | | |
| 719-720 | PEF | PEF protein with a long N-terminal | −6.6034 | −0.16368 | −0.088 | −0.01144 |
| | | hydrophobic domain (peflin) | | | | |
| 747-748 | LOX | lysyl oxidase | −6.4441 | −0.12784 | −0.077 | −0.02641 |
| 775-776 | IL1B | interleukin 1, beta | −6.4272 | −0.75203 | −0.637 | −0.52288 |
| 809-810 | MPHOSPH10 | M-phase phosphoprotein 10 (U3 small | −6.425 | −0.10347 | −0.048 | 0.007748 |
| | | nucleolar ribonucleoprotein) | | | | |
| 653-654 | NAGA | N-acetylgalactosaminidase, alpha- | −6.42 | −0.09205 | −0.034 | 0.024896 |
| 760-761 | MAPRE1 | microtubule-associated protein, | −6.39 | −0.39254 | −0.296 | −0.19928 |
| | | RP/EB family, member 1 | | | | |
| 811-812 | ARD1 | N-acetyltransferase, homolog of | −6.3833 | −0.1707 | −0.11 | −0.04915 |
| | | S. cerevisiae ARD1 | | | | |
| 813-814 | CDC6 | CDC6 (cell division cycle 6, | −6.371 | −0.25964 | −0.201 | −0.14327 |
| | | S. cerevisiae) homolog | | | | |
| 643 | EST | EST | −6.3541 | 0.133858 | 0.253 | 0.371068 |
| 583-584 | IL17R | IL-17 receptor | −6.3499 | −0.08991 | −0.035 | 0.019532 |
| 803 | WNT5B | wingless-type MMTV integration site | −6.3391 | −0.06803 | −0.017 | 0.035029 |
| | | family, member 5B | | | | |
| 651-652 | FDFT1 | farnesyl-diphosphate farnesyltransferase 1 | −6.3387 | −0.1152 | −0.038 | 0.039414 |
| 664-665 | EIF4A1 | eukaryotic translation initiation | −6.2705 | −0.39362 | −0.263 | −0.13253 |
| | | factor 4A, isoform 1 | | | | |
| 650 | EST | Unknown | −6.2573 | −0.08355 | −0.002 | 0.079181 |
| 657-658 | SLC9A1 | solute carrier family 9 (sodium/hydrogen | −6.2571 | −0.13608 | −0.077 | −0.01682 |
| | | exchanger), isoform 1 (antiporter, Na+/H+, | | | | |
| | | amiloride sensitive) | | | | |
| 731-732 | APMCF1 | APMCF1 protein | −6.2387 | −0.15677 | −0.083 | −0.00922 |
| 503 | ZNF220 | zinc finger protein 220 | −6.2316 | −0.13549 | −0.072 | −0.00922 |
| 815-816 | PTPRCAP | LPAP = lymphoid-restricted phospho- | −6.229 | −0.10182 | −0.059 | −0.01592 |
| | | protein = CD45 phosphatase binding | | | | |
| | | protein and putative substrate | | | | |
| 817 | POLD3 | polymerase (DNA directed), delta 3 | −6.223 | −0.29843 | −0.221 | −0.14327 |
| 788-789 | COVA1 | cytosolic ovarian carcinoma antigen 1 | −6.1802 | −0.42946 | −0.321 | −0.21325 |
| 701-702 | FLJ12442 | hypothetical protein FLJ12442 | −6.1607 | −0.07676 | −0.027 | 0.023252 |
| 818 | EST | Unknown | −6.1033 | −0.12494 | −0.057 | 0.011993 |
| 721-722 | EST | Human mRNA for unknown product, | −6.1032 | −0.40671 | −0.328 | −0.24949 |
| | | partial cds | | | | |
| 662-663 | HARS | histidyl-tRNA synthetase | −6.0889 | −0.24642 | −0.186 | −0.12552 |
| 819 | FLJ20746 | putative cyclin G1 interacting protein | −6.0827 | −0.04144 | 0.017 | 0.074451 |
| 820-821 | PLK | pLK = homologue of Drosophila polo | −6.0725 | −0.32422 | −0.253 | −0.18177 |
| | | serine/threonine kinase | | | | |
| 784 | TNRC12 | trinucleotide repeat containing 12 | 6.0744 | 0.110253 | 0.062 | 0.01368 |
| 723-724 | SAST | syntrophin associated serine/threonine | 6.0991 | 0.107549 | 0.065 | 0.022016 |
| | | kinase | | | | |
| 634-635 | EST | ESTs | 6.1178 | 0.048053 | −0.002 | −0.05306 |
| 695-696 | SMG1 | PI-3-kinase-related kinase SMG-1 | 6.1219 | 0.146438 | 0.087 | 0.028571 |
| 534-535 | LOC51760 | B/K protein | 6.1306 | 0.09691 | 0.048 | −0.00174 |
| 574-576 | IL7 | IL-7 | 6.1558 | 0.088845 | 0.043 | −0.00305 |
| 670-671 | PON1 | paraoxonase 1 | 6.3314 | 0.058046 | 0.014 | −0.03105 |
| 592-593 | ZFP161 | ZF5 = POZ domain zinc finger protein | 6.3356 | 0.196176 | 0.101 | 0.006466 |
| 632-633 | EST | ESTs, Moderately similar to | 6.3426 | 0.028978 | −0.029 | −0.08725 |
| | | ALU4_HUMAN ALU SUBFAMILY SB2 SEQUENCE | | | | |
| | | CONTAMINATION WARNING ENTRY [H. sapiens] | | | | |
| 765 | | ESTs | 6.4363 | 0.026533 | −0.03 | −0.08619 |
| 822 | CCR6 | CCR6 = STRL22 = chemokine receptor | 6.4897 | 0.116276 | 0.069 | 0.022016 |
| | | for MIP-3 alpha/LARC/Exodus on | | | | |
| | | activated B cells | | | | |
| 679-681 | ARHGEF6 | KIAA0006 | 6.5098 | 0.105169 | 0.065 | 0.024075 |
| 759 | LOC51605 | CGI-09 protein | 6.662 | 0.0086 | −0.042 | −0.09205 |
| 762 | FLJ10701 | hypothetical protein FLJ10701 | 6.925 | 0.135133 | 0.075 | 0.0141 |
| 636 | EST | Unknown | 7.3197 | 0.050766 | 0.005 | −0.04144 |
| 647 | EST | EST | 7.5484 | 0.045714 | −0.012 | −0.07007 |
| 725-726 | EST | Unknown | 8.058 | 0.068557 | −0.006 | −0.07988 |

TABLE 11
Results of Compound Covariate Predictor Analysis.

| | Pre-specified | Correctly |
|---|---|---|
| Expld | class label | classified |
| B2-1 vs OSE B2-1 vs OSE 21083 | 1 | YES |
| B2-10 vs OSE B2-10 vs OSE 21085 | 1 | YES |
| B2-16 vs OSE B2-16 vs OSE 21180 | 1 | YES |
| B2-2 vs OSE B2-2 vs OSE 21090 | 1 | YES |
| B2-20 vs OSE B2-20 vs OSE 21181 | 1 | YES |
| B2-21 vs OSE B2-21 vs OSE 21182 | 1 | YES |
| B2-22 vs OSE B2-22 vs OSE 21183 | 1 | YES |
| B2-23 vs OSE B2-23 vs OSE 21091 | 1 | YES |
| B2-24 vs OSE B2-24 vs OSE 21092 | 1 | NO |
| B2-25 vs OSE B2-25 vs OSE 22038 | 1 | YES |
| B2-3 vs OSE B2-3 vs OSE 21093 | 1 | YES |
| B2-4 vs OSE B2-4 vs OSE 21094 | 1 | YES |
| B2-5 vs OSE B2-5 vs OSE 21095 | 1 | NO |
| B2-7 vs OSE B2-7 vs OSE 21096 | 1 | YES |
| B2-8 vs OSE B2-8 vs OSE 21097 | 1 | YES |
| B2-9 vs OSE B2-9 vs OSE 21098 | 1 | YES |
| C100 vs OSE C100 vs OSE 21167 | 1 | YES |
| C102 vs OSE C102 vs OSE 21168 | 1 | YES |
| C103 vs OSE C103 vs OSE 21169 | 1 | YES |
| C105 vs OSE C105 vs OSE 21178 | 1 | YES |
| C107 vs OSE C107 vs OSE 21099 | 1 | YES |
| C110 vs OSE C110 vs OSE 21101 | 1 | YES |
| C111 vs OSE C111 vs OSE 21102 | 1 | NO |
| C117 vs OSE C117 vs OSE 21105 | 1 | YES |
| C118 vs OSE C118 vs OSE 21106 | 1 | YES |
| C123 vs OSE C123 vs OSE 21107 | 1 | YES |
| C46 vs OSE C46 vs OSE 19741 | 1 | NO |
| C77 vs OSE C77 vs OSE 21108 | 1 | YES |
| C84 vs OSE C84 vs OSE 21368 | 1 | YES |
| C85 vs OSE C85 vs OSE 21179 | 1 | YES |
| C99 vs OSE C99 vs OSE 21370 | 1 | YES |
| B36 vs OSE B36 vs OSE 19680 | 2 | YES |
| B39 vs OSE B39 vs OSE 19682 | 2 | YES |
| B40 vs OSE B40 vs OSE 19683 | 2 | YES |
| B41 vs OSE B41 vs OSE 19684 | 2 | YES |
| B52-2 vs OSE B52-2 vs OSE 19771 | 2 | NO |
| B54 vs OSE B54 vs OSE 19687 | 2 | YES |
| B55 vs OSE B55 vs OSE 19688 | 2 | YES |
| B60 vs OSE B60 vs OSE 19690 | 2 | YES |
| B61 vs OSE B61 vs OSE 19695 | 2 | YES |
| B62 vs OSE B62 vs OSE 19701 | 2 | YES |
| B63 vs OSE B63 vs OSE 19706 | 2 | YES |
| B64 vs OSE B64 vs OSE 19713 | 2 | YES |
| B70 vs OSE B70 vs OSE 19722 | 2 | YES |
| B74 vs OSE B74 vs OSE 19727 | 2 | YES |
| B77 vs OSE B77 vs OSE 19731 | 2 | YES |
| B78 vs OSE B78 vs OSE 21103 | 2 | YES |
| B79 vs OSE B79 vs OSE 19743 | 2 | YES |
| B80 vs OSE B80 vs OSE 21088 | 2 | YES |
| C114 vs OSE C114 vs OSE 21104 | 2 | YES |
| C15 vs OSE C15 vs OSE 19734 | 2 | YES |
| C16 vs OSE C16 vs OSE 19735 | 2 | YES |
| C17 vs OSE C17 vs OSE 19736 | 2 | YES |
| C1 vs OSE C1 vs OSE 19732 | 2 | YES |
| C20 vs OSE C20 vs OSE 19737 | 2 | YES |
| C41 vs OSE C41 vs OSE 19739 | 2 | YES |
| C42 vs OSE C42 vs OSE 19740 | 2 | YES |
| C49 vs OSE C49 vs OSE 19742 | 2 | YES |
| C79 vs OSE C79 vs OSE 21367 | 2 | YES |
| C87 vs OSE C87 vs OSE 19744 | 2 | YES |
| C95 vs OSE C95 vs OSE 21369 | 2 | YES |
| | Overall Success | 91.80% |

1. A method of classifying an ovarian tumor as a BRCA-1-like or BRCA-2-like or non-BRCA-like tumor, comprising:
determining a pattern of expression in the ovarian tumor of a plurality of markers listed in Table 1, wherein the pattern of expression in the ovarian tumor is determined relative to a standard ovarian tissue;
comparing a similarity of the pattern of expression of the plurality of markers in the ovarian tumor to a pattern of expression of the plurality of markers in a comparison tissue of a known BRCA-1-like or BRCA-2-like or non-BRCA-like tumor, wherein the pattern of expression in the comparison tissue is determined relative to the standard ovarian tissue;
wherein a similarity of the pattern of expression in the ovarian tumor to a pattern of expression of the comparison tissue of the known BRCA-1-like tumor classifies the ovarian tumor as a BRCA-1-like tumor, a similarity of the pattern of expression in the ovarian tumor to a pattern of expression of the known BRCA-2-like tumor classifies the ovarian tumor as a BRCA-2-like tumor, and a similarity of the pattern of expression in the ovarian tumor to a pattern of expression of the known non-BRCA-like tumor classifies the ovarian tumor as a non-BRCA-like tumor.

2. The method of claim 1, wherein the method comprises determining a pattern of over-expression or under-expression of the plurality of markers in the ovarian tumor to over-expression or under-expression of the plurality of markers of the comparison tissue.

3. The method of claim 2, wherein the method comprises determining a pattern of both over-expression and under-expression of the plurality of markers in the ovarian tumor to over-expression or under-expression of the plurality of markers in the comparison tissue.

4. The method of claim 1 wherein the comparison tissue is from a known BRCA-1-like tumor, and the method comprises determining whether the ovarian tumor is a BRCA-1-like tumor by comparing the pattern of expression in the ovarian tumor to the pattern of expression in the comparison tissue.

5. The method of claim 1 wherein the comparison tissue is from a subject known to have a mutation in BRCA-1 and the method comprises determining whether the ovarian tumor is a BRCA-1-like tumor by comparing the pattern of expression in the ovarian tumor to the pattern of expression in the comparison tissue.

6. The method of claim 1 wherein the comparison tissue is from a subject known to have a mutation in BRCA-2 and the method comprises determining whether the ovarian tumor is a BRCA-2-like tumor by comparing the pattern of expression in the ovarian tumor to the pattern of expression in the comparison tissue.

7. The method of claim 1 wherein the comparison tissue is from a known BRCA-2-like tumor, and the method comprises determining whether the ovarian tumor is BRCA-2-like by comparing the pattern of expression in the ovarian tumor to the pattern of expression in the comparison tissue.

8. The method of claim 7, wherein classifying the ovarian tumor comprises determining whether a tumor that does not contain a BRCA-1 or BRCA-2 mutation is BRCA-1-like or BRCA-2-like.

9. The method of claim 1 wherein the comparison tissue is from a known non-BRCA-like tumor, and the method comprises determining whether the ovarian tumor is non-BRCA-like by comparing the pattern of expression in the ovarian tumor to the pattern of expression in the comparison tissue.

10. The method of claim 1, wherein the standard ovarian tissue is tissue from an immortalized ovarian cell, ovarian tissue from a subject not having ovarian cancer, a subject not predisposed to developing ovarian cancer, or ovarian tissue from a subject from whom the ovarian tumor was obtained at an earlier point in time.

11. The method of claim 1, wherein the patterns of expression are patterns of logarithmic expression ratios.

12. The method of claim 1, wherein the patterns of expression are multidimensional scaling patterns.

13. The method of claim 12 wherein the multi-dimensional scaling patterns are visually compared to determine similarities.

14. The method of claim 1, wherein the patterns of expression are hierarchical clustering patterns.

15. The method of claim 14, wherein standard normal deviation values of the logarithmic expression ratios are assigned relative color intensities that assist in the visual comparison.

16. The method of claim 15, wherein the hierarchical clustering patters are visually compared to determine similarities.

17. The method of claim 11 comprising comparing the logarithmic expression ratios of the plurality markers using compound covariate predictor analysis.

18. The method of claim 11, wherein the method comprises differentiating a BRCA1-like ovarian tumor from a sporadic ovarian tumor by comparing relative logarithmic expression ratios of at least one marker shown in Table 6.

19. The method of claim 18, wherein differentiating a BRCA1-linked ovarian tumor from a sporadic ovarian tumor comprises comparing the relative logarithmic expression ratios of CD72 (SEQ ID NO: 805), SLC25A11 (SEQ ID NO: 544), LCN2 (SEQ ID NO: 545-547), PSTP1P1(SEQ ID NO: 538-540), SIAHBP1 (SEQ ID NO: 543), UBE1 (SEQ ID NO: 533), WAS (SEQ ID NO: 524-526), IDH2 (SEQ ID NO: 541-542), or PCTK1 (SEQ ID NO: 527-528) in the ovarian tumor and comparison tissue.

20. The method of claim 11, wherein the method comprises differentiating a BRCA2-like ovarian tumor from a non-BRCA-like ovarian tumor by comparing relative logarithmic expression ratios of at least one marker shown in Table 7.

21. The method of claim 20, wherein the method comprises comparing the relative logarithmic expression ratios of LOC51760 (SEQ ID NO: 279) or LRPAP1 (SEQ ID NO: 280) to differentiate a BRCA2-like ovarian tumor from a non-BRCA like ovarian tumor

22. The method of claim 21, wherein the method comprises differentiating a non-BRCA-like tumor from a BRCA-1-like or BRCA-2-like ovarian tumor by comparing relative logarithmic expression ratios of at least one marker shown in Table 8.

23. The method of claim 22, wherein the method comprises comparing relative logarithmic expression ratios of PSTP1P1 (SEQ ID NO: 281), IDH2 (SEQ ID NO: 282), or PCTK1 (SEQ ID NO: 274) to differentiate a combined BRCA1- and BRCA2-linked ovarian tumor from a sporadic ovarian tumor.

24. The method of claim 11, wherein the method comprises differentiating a BRCA1-like ovarian tumor from a BRCA2-like ovarian tumor by comparing relative logarithmic expression ratios of at least one marker shown in Table 10.

25. The method of claim 1, wherein the method further comprises selecting a treatment strategy based on classifying the ovarian tumor as BRCA1-like, BRCA2-like or non-BRCA-like.

26. The method of claim 25, wherein the treatment strategy comprises selecting a more aggressive treatment regimen for a BRCA1-like or BRCA2-like tumor.

27. The method of claim 26, wherein the treatment is chemotherapy, radiotherapy, or surgical removal of the affected tissue and/or surrounding area.

28. The method of claim 25, further comprising treating the subject with the selected treatment.

29. The method of claim 11, wherein comparing the patterns of logarithmic expression ratios comprises comparing the logarithmic expression ratios to patterns of logarithmic expression ratios in a database of patterns associated with BRCA1-like, BRCA2-like or non-BRCA-like ovarian tumors.

30. The method of claim 11, wherein comparing patterns of logarithmic expression ratios of the plurality of markers comprises obtaining the pattern of expression of the plurality of markers on an array.

31. The method of claim 1, wherein the pattern of expression of the plurality of markers comprises over-expression of one or more markers compared to the standard.

32. The method of claim 29, wherein the one or more markers that is overexpressed is listed in Table 5.

33. The method of claim 32, wherein determining the pattern of expression comprises providing nucleic acid sequences of the markers, and performing nucleic acid hybridization of specific oligonucleotide probes to the nucleic acid sequences.

34. The method of claim 33, wherein the sequence of the oligonucleotide probe is selected to bind specifically to a nucleic acid molecule listed in Table 1.

35. The method of claim 34, further comprising amplifying the one or more markers prior to performing nucleic acid hybridization.

36. The method of claim 33, further comprising quantitating hybridization to detect a level of differential expression.

37. The method of claim 33, wherein providing sequences of the markers comprises providing the nucleic acid sequences on an array carrying the plurality of markers.

38. The method of claim 37, wherein the array is a cDNA microarray.

39. The method of claim 33, wherein providing the nucleic acid sequences of the markers comprises providing at least 50 of the markers listed in Table 1.

40. The method of claim 33, wherein providing the nucleic acid sequences of the markers comprises providing at least 100 of the markers listed in Table 1.

41. The method of claim 33, wherein providing the nucleic acid sequences of the markers comprises providing at least 200 of the markers listed in Table 1.

42. A method of diagnosing or prognosing development or progression of ovarian cancer in a subject comprising detecting under-expression of one or more markers in Table 4 relative to a standard.

43. The method of claim 42, wherein the standard is immortalized ovarian epithelial cells, ovarian tissue from a subject not having cancer or a subject not predisposed to developing cancer, or ovarian tissue from the subject at an earlier point in time.

44. The method of claim 42 wherein the one or more markers comprise a nucleic acid encoded by SEQ ID NOs: 449-503.

45. A method of diagnosing or prognosing development or progression of ovarian cancer in a subject comprising detecting over-expression of one or more markers in Table 5 relative to a standard.

46. The method of claim 45, wherein the standard is immortalized ovarian epithelial cells, ovarian tissue from a subject not having cancer or a subject not predisposed to developing cancer, or ovarian tissue from the subject at an earlier point in time.

47. The method of claim 45 wherein the one or more markers comprise a nucleic acid encoded by SEQ ID NOs: 18-19, 30-31, 50-51, 52-54, 55-57, 58-59, 60, 68-69, 74-76, 85-86, 87-88, 89-91, 92-93, 94-95, 97-99, 122-123, 133-135, 149-151, 164-166, 167-168, 169-170, 174-175, 176-178, 179-180, 181-182, 190-192, or 199-201.

48. A method of screening for an agent for treating or inhibiting ovarian cancer in a subject, comprising exposing a tumor cell to a therapeutically effective amount of a pharmaceutical compound that restores wild-type expression of at least one BRCA1-like or BRCA2-like marker listed in Table 1.

49. The method of claim 48 wherein the agent corrects under-expression or over-expression of a marker listed in Table 1.

50. A method of monitoring a response to therapy for an ovarian tumor, comprising monitoring expression of the markers in the subject following administration of the therapy.

51. A method of diagnosing or prognosing development or progression of ovarian cancer in a subject comprising detecting differential expression of a gene that maps to Chromosome Xp11.2.

52. A kit for classifying one or more ovarian tumors as sporadic, BRCA1-like or BRCA2-like tumors, comprising components for measuring expression levels of markers in the one or more ovarian tumor samples and for comparing the expression levels of the markers to the markers in Table 10.

53. The kit of claim 52, wherein the expression levels of a plurality of markers from each tumor are measured.

54. The kit of claim 52, comprising an array carrying a plurality of markers.

55. The kit of claim 52, comprising a binding molecule that selectively binds to a marker in the one or more tumor samples, and wherein the marker is listed in Table 10.

56. The kit of claim 52, wherein the expression levels measured are of a non-BRCA-like, BRCA1-like or BRCA2-like tumor protein, and the binding molecule is an antibody or antibody fragment that selectively binds the tumor protein.

57. The kit of claim 52, wherein the expression levels measured are of a BRCA-like, BRCA1-like or BRCA2-like nucleic acid marker, and the binding molecule is an oligonucleotide capable of hybridizing to the nucleic acid molecule marker.
